SEQUENCE LISTING (62 sequences)

SEQ ID NO: 1 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSIYLDKQAAXEFVNWLLAGGPSSGAPPPS

SEQ ID NO: 2 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSIYLDKKAAXEFVNWLLAGGPSSGAPPPS

SEQ ID NO: 3 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSIYLDKQAAXKFVNWLLAGGPSSGAPPPS

SEQ ID NO: 4 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSIYLDKQAAXKFVQWLLEGGPSSGAPPPS

SEQ ID NO: 5 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSIYLEKQAAXKFVQWLLEGGPSSGAPPPS

SEQ ID NO: 6 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSIYLEKQAAXEFVNWLLAGGPSSGAPPPS

SEQ ID NO: 7 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSIYLEKQAAXEFVQWLLEGGPSSGAPPPS

SEQ ID NO: 8 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXHGTFTSDYSIYLEKQAAXEFVQWLLEGGPSSGAPPPS

SEQ ID NO: 9 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXQGTFTSDYSIYLEKQAAXEFVQWLLEGGPSSGAPPPS

SEQ ID NO: 10 - protein, 39 aa, Artificial
  Synthetic Polypeptide
  MISC_FEATURE (2)..(2): Xaa is Aib (alpha-aminoisobutyric acid)
YXQGTFTSDYSIYLEKQAAQEFVQWLLEGGPSSGAPPPS

SEQ ID NO: 11 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXHGTFTSDYSIYLEKQAAXEFVQWLLEGGPSSGAPPPS

SEQ ID NO: 12 - protein, 39 aa, Artificial
  Synthetic Polypeptide
  MISC_FEATURE (2)..(2): Xaa is Aib (alpha-aminoisobutyric acid)
HXHGTFTSDYSIYLEKQAAQEFVQWLLEGGPSSGAPPPS

SEQ ID NO: 13 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXHGTFTSDYSIYLEKQYAXEFVQWLLEGGPSSGAPPPS

SEQ ID NO: 14 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXHGTFTSDYSYYLEKQAAXEFVQWLLEGGPSSGAPPPS

SEQ ID NO: 15 - protein, 39 aa, Artificial
  Synthetic Polypeptide
  MISC_FEATURE (2)..(2): Xaa is Aib (alpha-aminoisobutyric acid)
HXHGTFTSDYSIYLEKQAAEEFVQWLLEGGPSSGAPPPS

SEQ ID NO: 16 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXHGTFTSDYSIYLDKQAAXEFVQWLLEGGPSSGAPPPS

SEQ ID NO: 17 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXHGTFTSDYSYYLEKQYAXEFVQWLLEGGPSSGAPPPS

SEQ ID NO: 18 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXHGTFTSDYSIYLEKKYAXEFVQWLLEGGPSSGAPPPS

SEQ ID NO: 19 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXHGTFTSDYSYYLEKKAAXEFVQWLLEGGPSSGAPPPS

SEQ ID NO: 20 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXHGTFTSDYSIYLEKKAAXEFVQWLLEGGPSSGAPPPS

SEQ ID NO: 21 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXHGTFTSDYSIYLEKQKAXEFVQWLLEGGPSSGAPPPS

SEQ ID NO: 22 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXHGTFTSDYSKYLEKQAAXEFVQWLLEGGPSSGAPPPS

SEQ ID NO: 23 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXHGTFTSDYSIYLEKRAAXEFVQWLLEGGPSSGAPPPS

SEQ ID NO: 24 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXHGTFTSDYSILLEKKYAXEFVQWLLEGGPSSGAPPPS

SEQ ID NO: 25 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXHGTFTSDYSYLLEKQAAXEFVQWLLEGGPSSGAPPPS

SEQ ID NO: 26 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
HXHGTFTSDYSILLEKQAAXEFVQWLLEGGPSSGAPPPS

SEQ ID NO: 27 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSILLEKQAAXEFVQWLLEGGPSSGAPPPS

SEQ ID NO: 28 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSYYLEKQAAXEFVQWLLEGGPSSGAPPPS

SEQ ID NO: 29 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSYLLEKQAAXEFVQWLLEGGPSSGAPPPS

SEQ ID NO: 30 - protein, 39 aa, Artificial
  Synthetic Polypeptide
  MISC_FEATURE (2)..(2): Xaa is Aib (alpha-aminoisobutyric acid)
HXHGTFTSDYSILLEKQAAEEFVQWLLEGGPSSGAPPPS

SEQ ID NO: 31 - protein, 39 aa, Artificial
  Synthetic Polypeptide
  MISC_FEATURE (2)..(2): Xaa is Aib (alpha-aminoisobutyric acid)
HXHGTFTSDYSILLEKQAAQEFVQWLLEGGPSSGAPPPS

SEQ ID NO: 32 - protein, 39 aa, Artificial
  Synthetic Polypeptide
  MISC_FEATURE (2)..(2): Xaa is Aib (alpha-aminoisobutyric acid)
HXHGTFTSDYSILLEKQAAREFVQWLLEGGPSSGAPPPS

SEQ ID NO: 33 - protein, 39 aa, Artificial
  Synthetic Polypeptide
  MISC_FEATURE (2)..(2): Xaa is Aib (alpha-aminoisobutyric acid)
HXHGTFTSDYSILLEKQAAHEFVQWLLEGGPSSGAPPPS

SEQ ID NO: 34 - protein, 42 aa, Mus musculus
  MISC_FEATURE: murine GIP
YAEGTFISDYSIAMDKIRQQDFVNWLLAQRGKKSDWKHNITQ

SEQ ID NO: 35 - protein, 31 aa, Homo sapiens
  MISC_FEATURE: human GLP-1(7-37)
HAEGTFTSDVSSYLEGQAAKEFIAWLVKGRG

SEQ ID NO: 36 - protein, 42 aa, Homo sapiens
  MISC_FEATURE: human GIP
YAEGTFISDYSIAMDKIHQQDFVNWLLAQKGKKNDWKHNITQ

SEQ ID NO: 37 - protein, 29 aa, Homo sapiens
  MISC_FEATURE: human Glucagon
HSQGTFTSDYSKYLDSRRAQDFVQWLMNT

SEQ ID NO: 38 - protein, 39 aa, Artificial
  Synthetic Polypeptide
  MISC_FEATURE (2)..(2): Xaa is Aib (alpha-aminoisobutyric acid)
YXQGTFTSDYSIYLDKQAAKEFVNWLLAGGPSSGAPPPS

SEQ ID NO: 39 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSKYLDKQAAXEFVNWLLAGGPSSGAPPPS

SEQ ID NO: 40 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSIKLDKQAAXEFVNWLLAGGPSSGAPPPS

SEQ ID NO: 41 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSIYKDKQAAXEFVNWLLAGGPSSGAPPPS

SEQ ID NO: 42 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSIYLDKQAAXEFVKWLLAGGPSSGAPPPS

SEQ ID NO: 43 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSIYLDKQAAXEFVNWLLAKGPSSGAPPPS

SEQ ID NO: 44 - protein, 39 aa, Artificial
  Synthetic Polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSIYLDKQAAXEFVNWLLAGGPSSGKPPPS

SEQ ID NO: 45 - protein, 39 aa, Artificial
  Synthetic Polypeptide
  MISC_FEATURE (1)..(1): Xaa is His or Tyr
  MISC_FEATURE (2)..(2): Xaa is Aib (alpha-aminoisobutyric acid)
  MISC_FEATURE (3)..(3): Xaa is His or Gln
  MISC_FEATURE (12)..(12): Xaa is Lys, Ile or Tyr
  MISC_FEATURE (13)..(13): Xaa is Leu or Tyr
  MISC_FEATURE (15)..(15): Xaa is Asp or Glu
  MISC_FEATURE (17)..(17): Xaa is Gln, Arg, or Lys
  MISC_FEATURE (18)..(18): Xaa is Ala, Tyr or Lys
  MISC_FEATURE (20)..(20): Xaa is Gln, Glu, Arg, His or Aib (alpha- aminoisobutyric acid)
  MISC_FEATURE (21)..(21): Xaa is Glu or Lys
  MISC_FEATURE (24)..(24): Xaa is Asn or Gln
  MISC_FEATURE (28)..(28): Xaa is Ala or Glu
XXXGTFTSDYSXXLXKXXAXXFVXWLLXGGPSSGAPPPS

SEQ ID NO: 46 - protein, 39 aa, Artificial; identical to SEQ ID NO: 45
  Synthetic Polypeptide
  MISC_FEATURE (1)..(1): Xaa is His or Tyr
  MISC_FEATURE (2)..(2): Xaa is Aib (alpha-aminoisobutyric acid)
  MISC_FEATURE (3)..(3): Xaa is His or Gln
  MISC_FEATURE (12)..(12): Xaa is Ile or Tyr
  MISC_FEATURE (13)..(13): Xaa is Leu or Tyr
  MISC_FEATURE (15)..(15): Xaa is Asp or Glu
  MISC_FEATURE (17)..(17): Xaa is Gln, Arg or Lys
  MISC_FEATURE (18)..(18): Xaa is Ala, Tyr or Lys
  MISC_FEATURE (20)..(20): Xaa is Gln, Glu, Arg, His or Aib (alpha- aminoisobutyric acid)
  MISC_FEATURE (21)..(21): Xaa is Glu or Lys
  MISC_FEATURE (24)..(24): Xaa is Asn or Gln
  MISC_FEATURE (28)..(28): Xaa is Ala or Glu

SEQ ID NO: 47 - protein, 39 aa, Artificial
  Synthetic Polypeptide
  MISC_FEATURE (2)..(2): Xaa is Aib (alpha-aminoisobutyric acid)
  MISC_FEATURE (12)..(12): Xaa is Ile or Tyr
  MISC_FEATURE (13)..(13): Xaa is Leu or Tyr
  MISC_FEATURE (15)..(15): Xaa is Asp or Glu
  MISC_FEATURE (17)..(17): Xaa is Gln or Lys
  MISC_FEATURE (18)..(18): Xaa is Ala or Tyr
  MISC_FEATURE (20)..(20): Xaa is Aib (alpha-aminoisobutyric acid)
  MISC_FEATURE (24)..(24): Xaa is Asn or Gln
  MISC_FEATURE (28)..(28): Xaa is Ala or Glu
HXHGTFTSDYSXXLXKXXAXEFVXWLLXGGPSSGAPPPS

SEQ ID NO: 48 - protein, 39 aa, Artificial
  Synthetic Polypeptide
  MISC_FEATURE (2)..(2): Xaa is Aib (alpha-aminoisobutyric acid)
  MISC_FEATURE (12)..(12): Xaa is Ile or Tyr
  MISC_FEATURE (13)..(13): Xaa is Leu or Tyr
  MISC_FEATURE (17)..(17): Xaa is Gln or Lys
  MISC_FEATURE (18)..(18): Xaa is Ala or Tyr
  MISC_FEATURE (20)..(20): Xaa is Aib (alpha-aminoisobutyric acid)
  MISC_FEATURE (24)..(24): Xaa is Asn or Gln
  MISC_FEATURE (28)..(28): Xaa is Ala or Glu
HXHGTFTSDYSXXLEKXXAXEFVXWLLXGGPSSGAPPPS

SEQ ID NO: 49 - protein, 39 aa, Artificial
  Synthetic Polypeptide
  MISC_FEATURE (2)..(2): Xaa is Aib (alpha-aminoisobutyric acid)
  MISC_FEATURE (12)..(12): Xaa is Ile or Tyr
  MISC_FEATURE (13)..(13): Xaa is Leu or Tyr
  MISC_FEATURE (17)..(17): Xaa is Gln or Lys
  MISC_FEATURE (18)..(18): Xaa is Ala or Tyr
  MISC_FEATURE (20)..(20): Xaa is Aib (alpha-aminoisobutyric acid)
HXHGTFTSDYSXXLEKXXAXEFVQWLLEGGPSSGAPPPS

SEQ ID NO: 50 - protein, 39 aa, Artificial
  Synthetic Polypeptide
  MISC_FEATURE (2)..(2): Xaa is Aib (alpha-aminoisobutyric acid)
  MISC_FEATURE (12)..(12): Xaa is Ile or Tyr
  MISC_FEATURE (13)..(13): Xaa is Leu or Tyr
  MISC_FEATURE (15)..(15): Xaa is Asp or Glu
  MISC_FEATURE (17)..(17): Xaa is Gln or Lys
  MISC_FEATURE (18)..(18): Xaa is Ala or Tyr
  MISC_FEATURE (20)..(20): Xaa is Aib (alpha-aminoisobutyric acid)
  MISC_FEATURE (24)..(24): Xaa is Asn or Gln
  MISC_FEATURE (28)..(28): Xaa is Ala or Glu
YXQGTFTSDYSXXLXKXXAXEFVXWLLXGGPSSGAPPPS

SEQ ID NO: 51 - protein, 39 aa, Artificial
  Synthetic Polypeptide
  MISC_FEATURE (2)..(2): Xaa is Aib (alpha-aminoisobutyric acid)
  MISC_FEATURE (12)..(12): Xaa is Ile or Tyr
  MISC_FEATURE (13)..(13): Xaa is Leu or Tyr
  MISC_FEATURE (17)..(17): Xaa is Gln or Lys
  MISC_FEATURE (20)..(20): Xaa is Aib (alpha-aminoisobutyric acid)
YXQGTFTSDYSXXLEKXAAXEFVQWLLEGGPSSGAPPPS

SEQ ID NO: 52 - protein, 39 aa, Artificial
  Synthetic Polypeptide
  MISC_FEATURE (2)..(2): Xaa is Aib (alpha-aminoisobutyric acid)
  MISC_FEATURE (12)..(12): Xaa is Ile or Tyr
  MISC_FEATURE (13)..(13): Xaa is Leu or Tyr
  MISC_FEATURE (17)..(17): Xaa is Gln or Lys
  MISC_FEATURE (20)..(20): Xaa is Aib (alpha-aminoisobutyric acid)
YXQGTFTSDYSXXLDKXAAXEFVNWLLAGGPSSGAPPPS

SEQ ID NO: 53 - protein, 40 aa, Artificial
  Synthetic polypeptide
  MISC_FEATURE (2)..(2): Xaa is Aib (alpha-aminoisobutyric acid)
  MISC_FEATURE (20)..(20): Xaa is Aib (alpha-aminoisobutyric acid)
YXQGTFTSDYSIYLDKQAAXEFVNWLLAGGPSSGAPPPSK

SEQ ID NO: 54 - protein, 5 aa, Artificial
  Synthetic
SYYLE

SEQ ID NO: 55 - protein, 5 aa, Artificial
  Synthetic
SILLE

SEQ ID NO: 56 - protein, 5 aa, Artificial
  Synthetic
SYLLE

SEQ ID NO: 57 - protein, 5 aa, Artificial
  Synthetic
SIYLE

SEQ ID NO: 58 - protein, 5 aa, Artificial
  Synthetic
SIYLD

SEQ ID NO: 59 - protein, 5 aa, Artificial
  Synthetic
  MISC_FEATURE (5)..(5): Xaa is Aib (alpha-aminoisobutyric acid)
KQAAX

SEQ ID NO: 60 - protein, 5 aa, Artificial
  Synthetic
  MISC_FEATURE (5)..(5): Xaa is Aib (alpha-aminoisobutyric acid)
KKAAX

SEQ ID NO: 61 - protein, 5 aa, Artificial
  Synthetic
  MISC_FEATURE (5)..(5): Xaa is Aib (alpha-aminoisobutyric acid)
KQYAX

SEQ ID NO: 62 - protein, 5 aa, Artificial
  Synthetic
  MISC_FEATURE (5)..(5): Xaa is Aib (alpha-aminoisobutyric acid)
KKYAX

(19) United States
(12) Patent Application Publication
(10) Pub. No.: US 2023/0391845 A1
(43) Pub. Date: Dec. 7, 2023

(54) GLP-1, GIP AND GLUCAGON RECEPTOR TRIPLE AGONISTS

(71) Applicant: Novo Nordisk A/S, Bagsvaerd (DK)

(72) Inventors: Patrick James Knerr, Plainfield, IN (US); Brian Patrick Finan, Indianapolis, IN (US); Yantao He, Fishers, IN (US); Richard Dimarchi, Carmel, IN (US); Fa Liu, Watchung, NJ (US); Bhavesh Premdjee, Frederiksberg (DK)

(21) Appl. No.: 18/033,572

(22) PCT Filed: Oct. 29, 2021
(86) PCT No.: PCT/EP2021/080089

§ 371 Date: Apr. 25, 2023

Related U.S. Application Data
(60) Provisional application No. 63/107,622, filed on Oct. 30, 2020.

(30) Foreign Application Priority Data
Dec. 10, 2020 (EP) 20213053.0

(51) Int. Cl. C07K 14/605; C07K 14/645

(52) U.S. Cl. CPC C07K 14/605; C07K 14/645; A61K 38/00

(57) ABSTRACT

The invention relates to peptides and derivatives thereof that are GLP-1/GIP/Glucagon receptor triple agonists and their medical use in treatment and/or prevention of obesity, diabetes, and/or liver diseases are described.

### TECHNICAL FIELD

The present invention relates to novel compounds that are agonists of the glucagon-like peptide 1 receptor (GLP-1R), the glucose-dependent insulinotropic polypeptide receptor (GIPR), and the glucagon receptor (GcgR) with a protracted profile of action.

### INCORPORATION-BY-REFERENCE OF THE SEQUENCE LISTING

The present application is filed with a Sequence Listing in electronic form. The entire contents of the sequence listing are hereby incorporated by reference. The sequence listing, entitled “200078WO01 sequence listing_ST25”, is 37,918 bytes and was created on 21 Oct. 2021.

### BACKGROUND

Glucagon-like peptide 1 (GLP-1) is a gut enteroendocrine cell-derived hormone and one of two prominent endogenous physiological incretins. GLP-1 improves glycemic control by stimulating glucose-dependent insulin secretion in response to nutrients (glucose), inhibits glucagon secretion from the pancreatic alpha-cells, slows gastric emptying, and induces body weight loss primary by decreasing food consumption. Glucose-dependent insulinotropic polypeptide (GIP), the other prominent incretin, improves glycemic control by stimulation of insulin secretion in response to nutrients (fat, glucose). Furthermore, GIP appears to improve plasma lipid profile and to stimulate calcium accumulation in bones. GIP analogues induce body weight loss and improve glycemic control by additive/synergistic action with GLP-1 analogues in dual administration and as single molecule co-agonists (Finan et al, Sci Transl Med, 2013, 5 (209): 209ra151; Frias et al, Cell Metab, 2017, 26 (2): 343-352; Coskun et al, Mol Metab, 2018, 18: 3-14; Frias et al, Lancet, 2018, 392 (10160): 2180-2193), and as such represent suitable candidates for amplification of GLP-1-based pharmacology.

Glucagon (Gcg) elevates hepatic glucose production by promoting glycogenolysis and gluconeogenesis to prevent occurrence of hypoglycaemia. In addition, glucagon has been shown to stimulate lipolysis, increase energy expenditure, and decrease hepatic lipoprotein production. Glucagon has been shown to provide additional weight loss when combined with GLP-1 than that achieved by GLP-1 alone (Evers et al, J Med Chem, 2017, 60 (10): 4293-4303; Henderson et al, Diabetes Obes Metab 2016, 18 (12): 1176-1190; Day et al, Nat Chem Biol, 2009, 5 (10): 749-757). This appears to be driven by the anorectic/satiety effects of GLP-1 combined with the thermogenic and lipolytic effects of glucagon. The insulinotropic and insulin-sensitizing effects of GLP-1 can counterbalance the potential diabetogenic liability of chronic glucagon action, as shown in clinical studies of two GLP-1/Gcg receptor co-agonists (Tillner et al, Diabetes Obes Metab, 2019, 21 (1): 120-128; Parker et al, J Clin Endocrinol Metab, 2019, 105 (3): 803-820). However, the ratio of GLP-1 activity to glucagon activity must be carefully controlled so as to maintain glycemic control, limiting the achievable weight lowering efficacy.

The limited therapeutic window of glucagon could be alleviated when combined with both GLP-1 and GIP, both of which acutely stimulate glucose-dependent insulin secretion and chronically enhance insulin-sensitivity. This dual incretin activity is predicted based upon mouse pharmacology to allow more aggressive glucagon pharmacology, driving additional efficacy with less diabetogenic liability. Additionally, the effects of glucagon on energy expenditure are not counteracted by GLP-1 nor GIP, which in turn provide their own anorectic effects. Thus, a triple agonist peptide is expected to provide glucose control accompanied by greater fat mass loss (Finan et al, Nat. Med., 2015, 21 (1): 27-36) than could be achieved with either GLP-1/GIP or GLP-1/Gcg co-agonism.

GLP-1/GIP/Gcg receptor triple agonists and their potential medical uses are described in several patent applications. Triple agonists with protraction appropriate for once-weekly administration in humans are described in WO 2019/125929, WO 2019/125938, and WO 2015/067716. Co-administration of protracted glucagon analogs with a protracted GLP-1/GIP co-agonist is described in WO 2017/009236. Triple agonists designed for protraction via conjugation to an Fc fragment are described in US 2019/0218269 and US 2019/0153060. Exendin-4 derivatives with triple agonistic activity and protraction appropriate for once-daily administration in humans are described in WO2017/009236, WO 2015/155141, WO 2014/096145, WO 2014/096148, and WO 2014/096150. However, no triple agonistic products have so far obtained market approval.

### SUMMARY

The invention relates to GLP-1/GIP/Gcg receptor triple agonists comprising a peptide and a substituent, wherein the substituent is covalently attached to the peptide in a position selected from the group consisting of positions 16, 17, and 21, e.g. the substituent is linked via an epsilon-amino group of lysine at a position selected from the group consisting of positions 16, 17, and 21 in the peptide. The substituent enhances half-life of the triple agonist and comprises a protractor A- comprising a di-fatty acid and a linker B-C-. The invention also relates to pharmaceutical compositions comprising such triple agonists and pharmaceutically acceptable excipients, as well as the medical use of said triple agonists.

In one aspect, the invention relates to a GLP-1/GIP/Gcg receptor triple agonist comprising a peptide and a substituent, wherein the amino acid sequence of the peptide is:

X₁X₂X₃GTFTSDYSX₁₂X₁₃LX₁₅KX₁₇X₁₈AX₂₀X₂₁FVX₂₄WLLX₂₈GGPSSGAPPPS (SEQ ID NO: 45), wherein

X₁ is H or Y

X₂ is Aib

X₃ is H or Q

X₁₂ is K, I or Y

X₁₃ is Y or L

X₁₅ is D or E

X₁₇ is Q, R, or K

X₁₈ is A, Y or K

X₂₀ is Aib, Q, E, R, or H

X₂₁ is E or K

X₂₄ is N or Q

X₂₈ is A or E;

wherein the peptide is a C-terminal acid or amide; and

wherein the substituent is covalently attached to the epsilon-amino group of the K in a position selected from the group consisting of positions 16, 17, and 21;

or pharmaceutically acceptable salts thereof.

Also or alternatively, in a second aspect, the invention relates to a pharmaceutical composition comprising the GLP-1/GIP/Gcg receptor triple agonists and optionally at least one pharmaceutically acceptable excipient.

Also or alternatively, in a further aspect, the invention relates to the GLP-1/GIP/Gcg receptor triple agonist as described herein for use as a medicament.

Also or alternatively, in a further aspect, the invention relates to the GLP-1/GIP/Gcg receptor triple agonist as described herein for use in prevention and/or treatment of diabetes, obesity and/or liver diseases.

### BRIEF DESCRIPTION OF DRAWINGS

FIG. 1 shows changes in body weight over time in a maximal efficacy study in DIO mice comparing vehicle (-□-); Compound No. 21 (-●-); Compound No. 30 (-Δ-); Compound No. 33 (-▾-). DIO mice received a subcutaneous dose once daily following the titration schedule described in Table 4.

FIG. 2 shows cumulative food intake in a maximal efficacy study in DIO mice comparing vehicle (-□-); Compound No. 21 (-●-); Compound No. 30 (-Δ-); Compound No. 33 (-▾-). DIO mice received a subcutaneous dose once daily following the titration schedule described in Table 4.

### DESCRIPTION

The present invention relates to compounds that are agonists at each of the receptors GLP-1, GIP, and Glucagon.

In what follows, Greek letters may be represented by their symbol or the corresponding written name, for example: α=alpha; β=beta; ε=epsilon; γ=gamma; ω=omega; etc. Also, the Greek letter of may be represented by “u”, e.g. in μl=ul, or in μM=uM.

Unless otherwise indicated in the specification, terms presented in singular form generally also include the plural situation.

Also described herein are triple agonists, pharmaceutical compositions and uses thereof in which open ended terms like “comprises” and “comprising” are replaced with closed terms such as “consists of”, “consisting of”, and the like.

### GLP-1/GIP/Glucagon Receptor Triple Agonist

The present invention relates to compounds that are agonists of the GLP-1 receptor, GIP receptor, and the glucagon receptor. Each of these agonists can also be referred to as a “GLP-1/GIP/Gcg receptor triple agonist” or a “triple agonist.” A receptor agonist as described herein is a compound that binds to a receptor and elicits a response of the natural ligand (see e.g. “Principles of Biochemistry”, AL Lehninger, DL Nelson, MM Cox, Second Edition, Worth Publishers, 1993, page 763).

In some embodiments, a GLP-1/GIP/Gcg triple agonist is a compound that binds to each of the three receptors GLP-1R, GIPR, and GcgR and elicits a response at each receptors, i.e. a compound which is capable of activating each of receptors GLP-1R, GIPR, and GcgR.

The term “compound” is used herein to refer to a molecular entity, and “compounds” may thus have different structural elements besides the minimum element defined for each compound or group of compounds. It follows that a compound may be a peptide or a derivative thereof, as long as the compound comprises the defined structural and/or functional elements.

The term “compound” is also meant to cover pharmaceutically relevant forms hereof, i.e. a compound as defined herein or a pharmaceutically acceptable salt, amide, or ester thereof.

The term “peptide” refers to a sequence of two or more amino acids. A “peptide” may also include amino acid elongations in the N-terminal and/or C-terminal positions and/or truncations in the N-terminal and/or C-terminal positions. In general, amino acid residues may be identified by their full name, their one-letter code, and/or their three-letter code. These three ways are fully equivalent.

Amino acids are molecules containing an amino group and a carboxylic acid group, and, optionally, one or more additional groups, often referred to as a side chain.

The term “amino acid” includes proteinogenic (or natural) amino acids (amongst those the 20 standard amino acids), as well as non-proteinogenic (or non-natural) amino acids. Proteinogenic amino acids are those which are naturally incorporated into proteins. The standard amino acids are those encoded by the genetic code. Non-proteinogenic amino acids are either not found in proteins, or not produced by standard cellular machinery (e.g., they may have been subject to post-translational modification). Non-limiting examples of non-proteinogenic amino acids are Aib (a-aminoisobutyric acid, or 2-aminoisobutyric acid), norleucine (Nle), norvaline as well as the D-isomers of the proteinogenic amino acids.

In what follows, each amino acid of the peptides for which the optical isomer is not stated is to be understood to mean the L-isomer (unless otherwise specified).

The GLP-1/GIP/Gcg triple agonists described herein comprise or consists of a peptide and a substituent. In some embodiments, the peptide is a synthetic peptide created to optimize the activity at the GLP-1, GIP, and Gcg receptors. Compounds having receptor activity towards each of the GLP-1, GIP and Gcg receptors, have been identified as demonstrated in the examples herein.

The triple agonists described herein further display an extended half-life gained by the substituent comprising a fatty acid.

In some embodiments, the carboxy terminus of the triple agonist holds a carboxylic acid group (—COOH), also referred to as a C-terminal acid. In some embodiments, the carboxy terminus of the triple agonist may optionally include an amide group (C(═O)—NH₂), also referred to as a C-terminal amide, which is a naturally occurring modification in some proteins substituting —OH with —NH₂, such as seen with native Exendin-4.

### Peptide

The GLP-1/GIP/Gcg receptor triple agonists described herein comprise a peptide and a substituent wherein the substituent is attached to the peptide backbone via an amino acid residue, e.g. via a functional group on an amino acid side chain. In some embodiments, the triple agonists described herein consists of a peptide and a substituent.

In some embodiments, the amino acid sequence of the peptide is: X₁X₂X₃GTFTSDYSX₁₂X₁₃LX₁₅KX₁₇X₁₈AX₂₀X₂₁FVX₂₄WLLX₂₈GGPSSGAPPPS (SEQ ID NO: 45), wherein

X₁ is H or Y

X₂ is Aib

X₃ is H or Q

X₁₂ is K, I or Y

X₁₃ is Y or L

X₁₅ is D or E

X₁₇ is Q, R, or K

X₁₈ is A, Y or K

X₂₀ is Aib, Q, E, R, or H

X₂₁ is E or K

X₂₄ is N or Q

X₂₈ is A or E;

wherein the peptide is a C-terminal acid or amide.

In some embodiments, the amino acid sequence of the peptide is:

(SEQ ID NO: 47)
HX₂HGTFTSDYSX₁₂X₁₃LX₁₅KX₁₇X₁₈AX₂₀EFVX₂₄WLLX₂₈GGPSSGAPPPS,

wherein

X₂ is Aib

X₁₂ is I or Y

X₁₃ is Y or L

X₁₅ is D or E

X₁₇ is Q or K

X₁₈ is A or Y

X₂₀ is Aib

X₂₄ is N or Q

X₂₈ is A or E.

In some embodiments, the amino acid sequence of the peptide is:

(SEQ ID NO: 50)
YX₂QGTFTSDYSX₁₂X₁₃LX₁₅KX₁₇X₁₈AX₂₀EFVX₂₄WLLX₂₈GGPSSGAPPPS,

wherein

X₂ is Aib

X₁₂ is I or Y

X₁₃ is Y or L

X₁₅ is D or E

X₁₇ is Q or K

X₁₈ is A or Y

X₂₀ is Aib

X₂₄ is N or Q

X₂₈ is A or E.

In some embodiments, X₁X₂X₃ are selected from the group consisting of HAibH and YAibQ. In some embodiments, X₁X₂X₃ is HAibH. In some embodiments, X₁X₂X₃ is YAibQ.

In some embodiments, X₁₂ is I or Y. In some embodiments, X₁₅ is D. In some embodiments, X₁₅ is E. In some embodiments, SX₁₂X₁₃LX₁₅ are selected from the group consisting of SYYLE (SEQ ID NO: 54), SILLE (SEQ ID NO: 55), SYLLE (SEQ ID NO: 56), SIYLE (SEQ ID NO. 57), and SIYLD (SEQ ID NO: 58). In some embodiments, SX₁₂X₁₃LX₁₅ are selected from the group consisting of SYYLE, SILLE, SIYLE, and SIYLD. In some embodiments, SX₁₂X₁₃LX₁₅ are selected from the group consisting of SYYLE and SILLE.

In some embodiments, KX₁₇X₁₈AX₂₀ are selected from the group consisting of KQAAAib (SEQ ID NO: 59), KKAAAib (SEQ ID NO: 60), KQYAAib (SEQ ID NO. 61), and KKYAAib (SEQ ID NO: 62). In some embodiments, KX₁₇X₁₈AX₂₀ is KQAAAib.

In some embodiments, X₂₄ is N. In some embodiments, X₂₄ is Q.

In some embodiments, X₂₈ is A. In some embodiments, X₂₈ is E.

In some embodiments, the peptide is selected from any one of SEQ ID NO: 1-33. In some embodiments, the amino acid sequence of the peptide is selected from the group consisting of SEQ ID NOs: 1, 11, 13-14, 17-18, 20, and 26. In some embodiments, the amino acid sequence of the peptide is selected from the group consisting of SEQ ID NO: 11, 14 and 26.

In some embodiments, the peptide is a C-terminal acid or amide. In some embodiments, the peptide is a C-terminal acid. In some embodiments, the peptide is a C-terminal amide.

### Derivatives

In some embodiments, the GLP-1/GIP/Gcg receptor triple agonists comprise a substituent covalently linked to a peptide. Such compounds may also be referred to as derivatives of the peptide, as they are obtained by covalently linking a substituent to a peptide backbone, e.g. via a functional group on an amino acid side chain. In some embodiments, the GLP-1/GIP/Gcg receptor triple agonists consist of a peptide and a substituent.

An aspect of the invention relates to a compound comprising a peptide and a substituent, wherein the amino acid sequence of the peptide is:

X₁X₂X₃GTFTSDYSX₁₂X₁₃LX₁₅KX₁₇X₁₈AX₂₀X₂₁FVX₂₄WLLX₂₈GGPSSGAPPPS (SEQ ID NO: 45), wherein

X₁ is H or Y

X₂ is Aib

X₃ is H or Q

X₁₂ is K, I or Y

X₁₃ is Y or L

X₁₅ is D or E

X₁₇ is Q, R, or K

X₁₈ is A, Y or K

X₂₀ is Aib, Q, E, R, or H

X₂₁ is E or K

X₂₄ is N or Q

X₂₈ is A or E;

wherein the peptide is a C-terminal acid or amide; and
and wherein the substituent is covalently attached to the epsilon-amino group of the K in a position selected from the group consisting of positions 16, 17, and 21; or pharmaceutically acceptable salts thereof.

In further embodiments, the peptide may be defined as described herein above.

### Substituent

In some embodiments, a substituent is covalently attached to the peptide backbone of the triple agonists as described herein, e.g. via the epsilon-amino group of a lysine (K) residue. In some embodiments, the substituent is covalently attached via the epsilon-amino group of a lysine (K) in a position selected from the group consisting of positions 16, 17, and 21 of the backbone. In a preferred embodiment, the substituent is covalently attached via the epsilon-amino group of the lysine (K) at position 16.

In one aspect, the substituent is capable of forming non-covalent complexes with proteins, e.g. albumin or serum albumin, thereby promoting the circulation of the triple agonist in the blood stream, and also having the effect of protracting the time of action of the triple agonist, due to the fact that the complex of the triple agonist and albumin is only slowly removed by renal clearance.

In some embodiments, the substituent is covalently attached to a lysine residue in the peptide backbone by acylation, i.e. via an amide bond formed between a carboxylic acid group of the substituent and the epsilon-amino group of said lysine. In some embodiments, the amino group of said lysine is coupled to an aldehyde of the substituent by reductive amination.

In some embodiments, the substituent is A-B-C-, comprising a protractor (A-) and a linker element (B-C-). In some embodiments, the protractor is a fatty acid and is the terminal part of the substituent responsible for extending half-life of the compound.

In some embodiments, the protractor, A-, is a fatty acid, such as a fatty diacid that may be defined by Chem. 1:

HOOC—(CH₂)ₚ—CO—* Chem. 1:

wherein p is an integer in the range of 8-20. In some embodiments, A- is Chem. 1 and p is an integer in the range of 14-20, such as 16 or 18. Chem. 1 may also be referred to as a C(n+2) diacid.

In some embodiments, the substituent further comprises a linker element B-C-, wherein B- is selected from Chem. 2 or Chem. 3:

*—NH—CH(COOH)—(CH₂)₂—CO—* Chem. 2:

Chem. 2 may also be referred to as γGlu which may also be described by Chem. 2a:

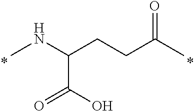

and include both R- and S-forms. In some embodiments, Chem. 2 is the S-isomer of γGlu.

*—NH—CH₂—(C₆H₁₀)—CO—NH—CH(COOH)—(CH₂)₂—CO—* Chem. 3:

which may also be described by Chem. 3a or Chem. 3b:

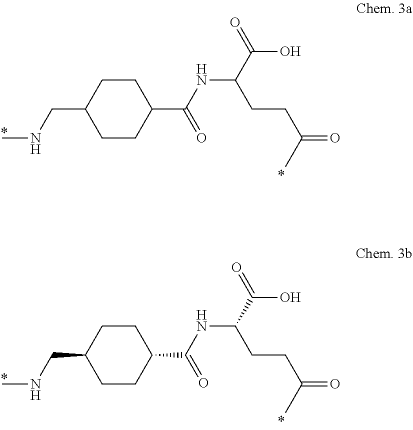 [SMILES: CNCC1CCC(C(=O)NC(CCC(C)=O)C(=O)O)CC1 ; CNC[C@H]1CC[C@H](C(=O)N[C@@H](CCC(C)=O)C(=O)O)CC1]

Chem. 3 may also be referred to as Trx-γGlu, wherein Trx refers to Tranexamic acid or trans-4-(aminomethyl)cyclohexanecarboxylic acid, where Chem. 3 covers the (1,2), (1,3), and (1,4) forms, while Chem. 3a and Chem. 3b specifies the (1,4) form.

In some embodiments, the substituent further comprises a linker element that may be defined by B-C-, wherein C- is selected from Chem. 4 or Chem. 5:

*—[NH—((CH₂)₂—O)₂—CH₂—CO]₂—*, Chem. 4:

which may also be referred to as Ado-Ado consisting of two 8-amino-3,6-dioxaoctanoic acid moieties linked via an amide bond, which may also be described by:

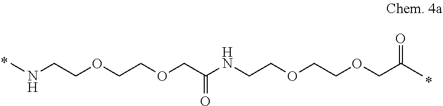 [SMILES: CNCCOCCOCC(=O)NCCOCCOCC(C)=O]

*—[NH—(CH₂)₄—CH(NH₂)—CO]₂—*, Chem. 5:

which may also be referred to a εLys-εLys consisting of two epsilon Lysine moieties linked via an amide bond, which may also be described by:

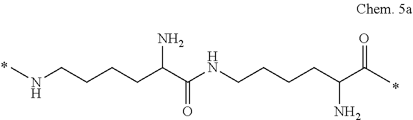 [SMILES: CNCCCCC(N)C(=O)NCCCCC(N)C(C)=O]

and include both R- and S-forms. In one embodiment, Chem. 5 is the S-isomer of both εLys moieties.

The symbol * denotes the points of attachment and A-, B-, and C- are interconnected by amide bonds in the sequence indicated.

In some embodiments, the substituent A-B-C- is selected from the group consisting of:

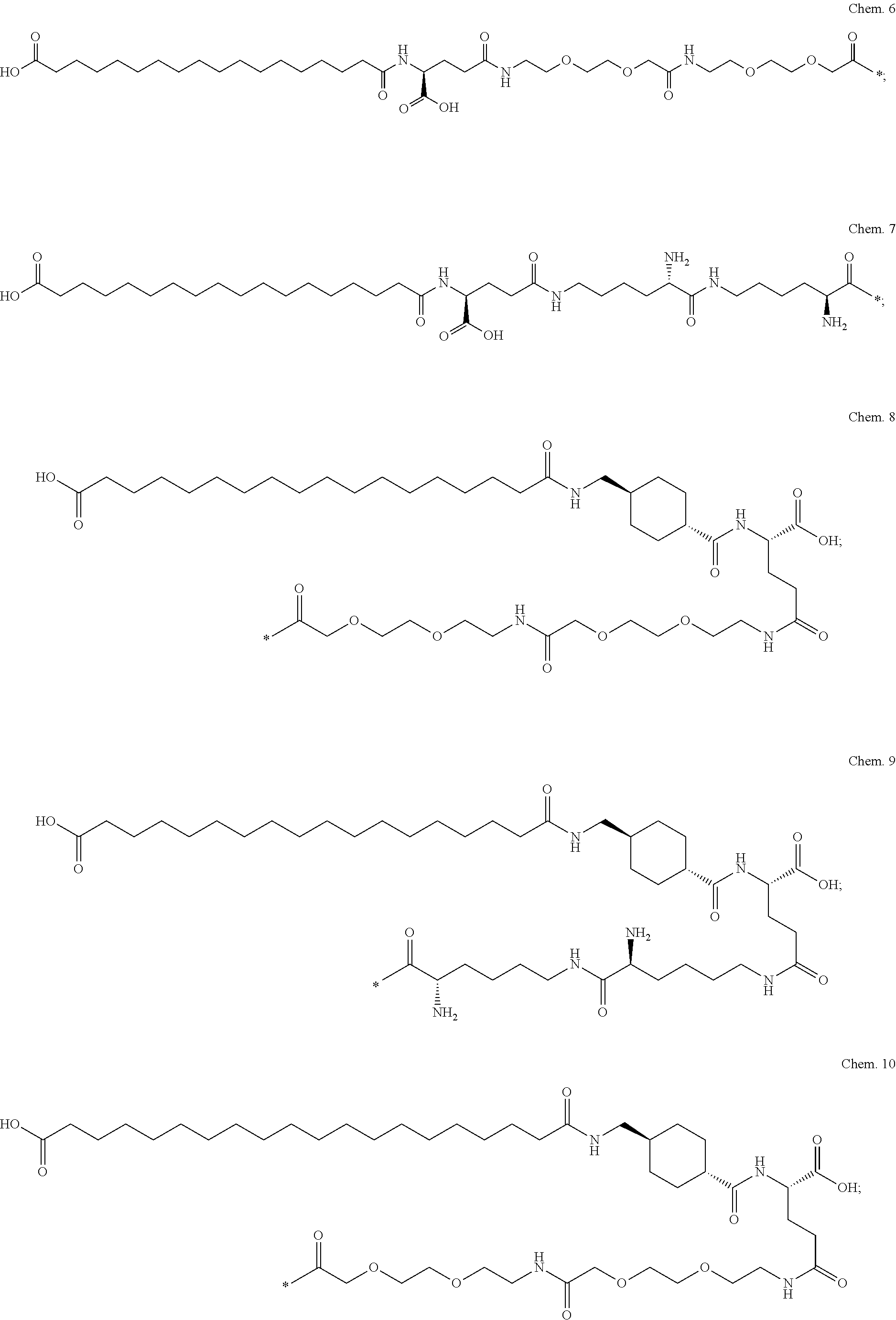 [SMILES: CC(=O)COCCOCCNC(=O)COCCOCCNC(=O)CC[C@H](NC(=O)CCCCCCCCCCCCCCCCC(=O)O)C(=O)O ; CC(=O)[C@@H](N)CCCCNC(=O)[C@@H](N)CCCCNC(=O)CC[C@H](NC(=O)CCCCCCCCCCCCCCCCC(=O)O)C(=O)O ; CC(=O)COCCOCCNC(=O)COCCOCCNC(=O)CC[C@H](NC(=O)[C@H]1CC[C@H](CNC(=O)CCCCCCCCCCCCCCCCC(=O)O)CC1)C(=O)O ; CC(=O)[C@@H](N)CCCCNC(=O)[C@@H](N)CCCCNC(=O)CC[C@H](NC(=O)[C@H]1CC[C@H](CNC(=O)CCCCCCCCCCCCCCCCC(=O)O)CC1)C(=O)O ; CC(=O)COCCOCCNC(=O)COCCOCCNC(=O)CC[C@H](NC(=O)[C@H]1CC[C@H](CNC(=O)CCCCCCCCCCCCCCCCCCC(=O)O)CC1)C(=O)O]

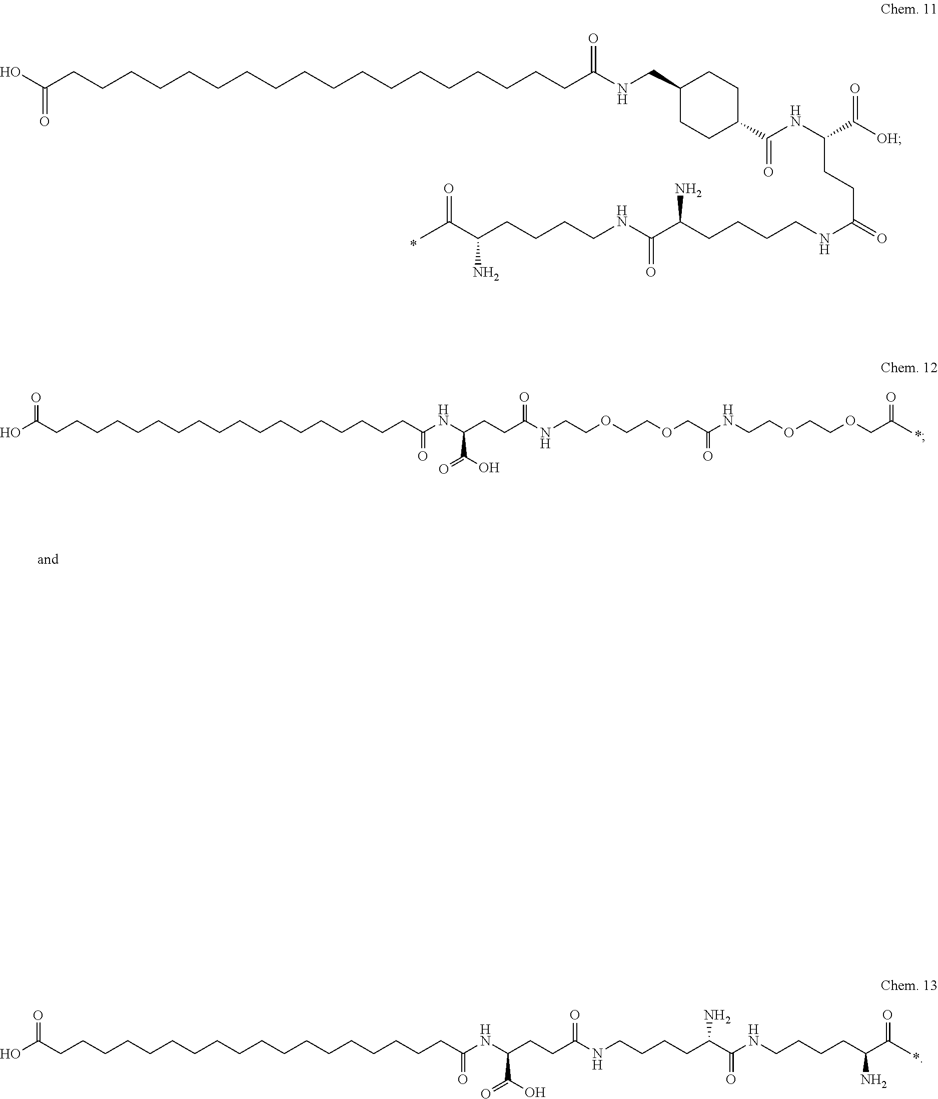 [SMILES: CC(=O)[C@@H](N)CCCCNC(=O)[C@@H](N)CCCCNC(=O)CC[C@H](NC(=O)[C@H]1CC[C@H](CNC(=O)CCCCCCCCCCCCCCCCCCC(=O)O)CC1)C(=O)O ; CC(=O)COCCOCCNC(=O)COCCOCCNC(=O)CC[C@H](NC(=O)CCCCCCCCCCCCCCCCCCC(=O)O)C(=O)O ; CC(=O)[C@@H](N)CCCCNC(=O)[C@@H](N)CCCCNC(=O)CC[C@H](NC(=O)CCCCCCCCCCCCCCCCCCC(=O)O)C(=O)O]

In some embodiments, the substituent is selected from the group consisting of Chem. 6, Chem. 7, Chem. 12, and Chem. 13. In a particular embodiment, the substituent A-B-C- is selected from Chem. 12 or Chem. 13.

### Pharmaceutically Acceptable Salts

In some embodiments, the triple agonists described herein are in the form of pharmaceutically acceptable salts.

Salts are e.g. formed by a chemical reaction between a base and an acid, e.g.: 2NH₃+H₂SO₄→(NH₄)₂SO₄.

The salt may be a basic salt, an acid salt, or it may be neither (i.e. a neutral salt). Basic salts produce hydroxide ions and acid salts hydronium ions in water.

In some embodiments, the salts of the triple agonists of the invention are formed with added cations or anions between anionic or cationic groups, respectively. These groups may be situated in the peptide moiety, and/or in the side chain of the triple agonists.

Non-limiting examples of anionic groups of the triple agonists described herein include free carboxylic groups in the side chain and free carboxylic groups in the peptide moiety. In some embodiments, the peptide moiety includes a free carboxylic acid group at the C-terminus. In some embodiments, the peptide moiety includes free carboxylic groups at internal acid amino acid residues, such as Asp and Glu. In some embodiments, the triple agonists described herein do not include a free carboxylic group.

Non-limiting examples of cationic groups in the peptide moiety include the free amino group at the N-terminus and a free amino group of internal basic amino acid residues, such as His, Arg, and Lys. In some embodiments, the triple agonists described herein do not include a free amino group at the N-terminus.

In a particular embodiment, the triple agonist is in the form of a pharmaceutically acceptable salt.

### Functional Properties

In a first aspect, the triple agonists as described herein are agonists at all of GLP-1R, GIPR, and GcgR as tested by in vitro potency as described in Example 2 herein. The triple agonists described herein can therefore activate each of the GLP-1R, GIPR, and GcgR.

Also, or alternatively, in a second functional aspect, the triple agonists have an in vivo effect on body weight, food intake and glucose tolerance.

Also, or alternatively, in a third functional aspect, the triple agonists produce improved pharmacokinetic properties over native hormones GLP-1, GIP and Gcg.

### Biological Activity—In Vitro Potency

In this section, the term “potency” is used interchangeably with “biological activity” and “activity”.

According to the first functional aspect, the triple agonists described herein are potent at the GLP-1R, the GIPR, and the GcgR. The peptides described herein also are potent at the GLP-1R, the GIPR, and the GcgR. In some embodiments, the triple agonists as described herein have potent in vitro effects to activate hGLP-1R, hGIPR and hGcgR.

In some embodiments, potency is determined by in vitro potency, i.e. performance of the triple agonists as described herein (or of the peptides as described herein) in a functional receptor assay for each of GLP-1R, GIPR, and GcgR. In some embodiments, the functional receptor assays determine the in vitro potency of the triple agonists as described herein (or of the peptides as described herein) for each of hGLP-1R, hGIPR and hGcgR individually.

The term “half maximal effective concentration” (also known as “EC₅₀”) refers to the concentration which induces a response halfway between the baseline and maximum, by reference to the dose response curve. EC₅₀ is used to measure the potency of a compound and represents the concentration where 50% of its maximal effect is observed.

The in vitro potency of the triple agonists as described herein may thus be determined as described below, by determining the EC₅₀. The lower the EC₅₀ value, the better potency. In order to characterize such compounds further, it may be relevant to consider the in vitro potencies relative to the native hormones of each receptor, i.e. hGLP-1(7-37) (SEQ ID NO: 35), hGIP (SEQ ID NO: 36), and hGcg (SEQ ID NO: 37).

The in vitro potency may, e.g. be determined in a medium containing membranes expressing the appropriate receptor, and/or in an assay with whole cells expressing the appropriate receptor.

For example, the functional response of the human GLP-1, GIP, or Gcg receptor may be measured in a reporter gene assay, e.g. in a stably transfected BHK cell line that expresses the human or mouse GLP-1, GIP, or Gcg receptor and contains the DNA for the cAMP response element (CRE) coupled to a promoter and the gene for firefly luciferase (CRE luciferase). When cAMP is produced as a result of activation of the GLP-1, GIP, or Gcg receptor, this in turn results in luciferase being expressed. Luciferase may be determined by adding luciferin, which by the enzyme is converted to oxyluciferin and produces bioluminescence, which is measured as a reporter of the in vitro potency. One non-limiting example of such an assay is described in Example 2 as described herein.

In some embodiments, the triple agonists as described herein are capable of activating the hGLP-1R, the hGIPR, and the hGcgR. In some embodiments, the triple agonists as described herein are capable of activating the hGLP-1R, hGIPR, and hGcgR in vitro with an EC₅₀ of less than 500 pM, such as less than 100 pM, such as less than 50 pM, in the CRE luciferase reporter gene assay as described in Example 2 herein.

In some embodiments, the triple agonists as described herein have an in vitro potency at the hGLP-1R, hGIPR, and hGcgR as determined using the method of Example 2 corresponding to an EC₅₀ at or below 200 pM, more preferably below 75 pM, or most preferably below 50 pM.

In one embodiment, the EC₅₀ in human GLP-1, GIP and Gcg receptor assays as described in Example 2 are 0.5-500 pM, such as 0.5-100 pM, or such as 0.5-50 pM.

### Biological Activity—Pharmacodynamic Study in DIO Mice

According to the second functional aspect, the triple agonists as described herein, are biological active in vivo, which may be determined by any technique known in the art in any suitable animal model, as well as in clinical trials.

The diet-induced obese (DIO) mouse is one example of a suitable animal model, and the effect on body weight, food intake, and glucose tolerance can be assessed during sub-chronic dosing in this model. The effect of the triple agonists as described herein on body weight, food intake and glucose tolerance may be determined in such mice in vivo, e.g. as described in Example 4 herein.

In some embodiments, the triple agonists as described herein display the ability to reduce body weight, food intake, and improve glucose tolerance in DIO mice as described in Example 4.

In some embodiments, the triple agonists as described herein reduce body weight in DIO mice.

In some embodiments, the triple agonists as described herein reduce food intake in DIO mice.

In some embodiments, the triple agonists as described herein improve glucose tolerance in DIO mice.

In some embodiments, the triple agonists as described herein reduce body weight by at least 10% after once daily administration of 1 nmol/kg of said compound, or by at least 25% after once daily administration of 3 nmol/kg of said compound, for 13 days in DIO mice. In some embodiments, the triple agonists as described herein reduce food intake by at least 20% compared to vehicle after once daily administration of 1 nmol/kg of said compound, or by at least 50% compared to vehicle after once daily administration of 3 nmol/kg of said compound, for 13 days in DIO mice. In some embodiments, the triple agonists improve glucose tolerance by at least 30% compared to vehicle after once daily administration of 1 nmol/kg or 3 nmol/kg of said compound for 13 days in DIO mice as measured in an IPGTT (intraperitoneal glucose tolerance test).

### Pharmacokinetics Profile—Half-Life In Vivo in Minipigs

According to the third functional aspect, the triple agonists as described herein, have improved pharmacokinetic properties such as increased terminal half-life e.g. as compared to native hormones GLP-1, GIP and Gcg. Increasing terminal half-life means that the compound in question is eliminated slower from the body. For the triple agonists as described herein this entails an extended duration of the pharmacological effect.

The pharmacokinetic properties of the triple agonists as described herein may suitably be determined in vivo in pharmacokinetic (PK) studies. Such studies are conducted to evaluate how pharmaceutical compounds are absorbed, distributed, and eliminated in the body, and how these processes affect the concentration of the triple agonist in the body, over the course of time.

In the discovery and preclinical phase of pharmaceutical drug development, animal models such as the mouse, rat, monkey, dog, or pig, may be used to perform PK studies. Any such models can be used to test PK properties of the triple agonist described herein.

In such studies, animals are typically administered with a single dose of the drug, either intravenously (i.v.), subcutaneously (s.c.), or orally (p.o.) in a relevant formulation. Blood samples are drawn at predefined time points after dosing, and samples are analysed for concentration of drug with a relevant quantitative assay. Based on these measurements, time-plasma concentration profiles for the compound of study are plotted and a so-called non-compartmental pharmacokinetic analysis of the data is performed.

Terminal half-life is an important parameter as a long half-life indicates that less frequent administration of a compound may be possible. In some embodiments, the terminal half-life is half-life (t½) in vivo in minipigs after i.v. administration, e.g. as described in Example 3 herein.

In some embodiments, the terminal half-life in minipigs of the triple agonists as described herein is at least 30 hours, preferably at least 40 hours, even more preferably at least 60 hours as measured after i.v. administration.

### Production Processes

The triple agonists as described herein, or a fragment thereof, may be produced by classical peptide synthesis, e.g., solid phase peptide synthesis using t-Boc or Fmoc chemistry or other well established techniques, see e.g., Greene and Wuts, “Protective Groups in Organic Synthesis”, John Wiley & Sons, 1999, Florencio Zaragoza Dörwald, “Organic Synthesis on solid Phase”, Wiley-VCH Verlag GmbH, 2000, and “Fmoc Solid Phase Peptide Synthesis”, Edited by W. C. Chan and P. D. White, Oxford University Press, 2000. In some embodiments, methods for preparing the triple agonists are described herein. In some embodiments, the methods for preparing the triple agonists as described herein comprises a step of solid phase peptide synthesis.

Also, or alternatively, the triple agonists as described herein may be produced by recombinant methods, viz. by culturing a host cell containing a DNA sequence encoding the triple agonist peptide sequence and capable of expressing the peptide in a suitable nutrient medium under conditions permitting the expression of the peptide. Non-limiting examples of host cells suitable for expression of peptides are: Escherichia coli, Saccharomyces cerevisiae, as well as mammalian BHK or CHO cell lines.

The triple agonists as described herein which include non-natural amino acids may e.g. be produced as described under ‘General Methods of Preparation’ in the experimental part. Or see e.g., Hodgson et al: “The synthesis of peptides and proteins containing non-natural amino acids”, Chemical Society Reviews, vol. 33, no. 7 (2004), p. 422-430.

The triple agonists as described herein which include a substituent may, e.g. be produced as described under ‘General Methods of Preparation’ in the experimental part. In some embodiments, the substituent is built as part of the solid phase peptide synthesis or built separately and attached via the lysine residue after the solid phase peptide synthesis.

Specific examples of methods of preparing a number of the triple agonists as described herein are provided below.

### Pharmaceutical Compositions

Pharmaceutical compositions comprising a triple agonist as described herein or a pharmaceutically acceptable salt thereof, and optionally one or more pharmaceutically acceptable excipients may be prepared as is known in the art.

The term “excipient” broadly refers to any component other than the active therapeutic ingredient(s). The excipient may be an inert substance, an inactive substance, and/or a non-medicinally active substance.

The excipient may serve various purposes, e.g. as a carrier, vehicle, diluent, tablet aid, and/or to improve administration, and/or absorption of the active substance.

The formulation of pharmaceutically active ingredients with various excipients is known in the art, see e.g. Remington: The Science and Practice of Pharmacy (e.g. 19th edition (1995), and any later editions).

In some embodiments, the pharmaceutical composition comprising the triple agonist as described herein is a liquid formulation, such as an aqueous formulation.

Liquid formulations, e.g. suitable for injection, can be prepared using conventional techniques of the pharmaceutical industry which involve dissolving and mixing the ingredients as appropriate to give the desired end product.

In some embodiments, the triple agonists as described herein are dissolved in a suitable buffer at a suitable pH so precipitation is minimised or avoided. The composition for injection may be sterilized, for example, by sterile filtration.

### Pharmaceutical Indications

A further aspect of the invention relates to the GLP-1/GIP/Gcg receptor triple agonist as described herein for use as a medicament.

In particular aspects of the invention, the GLP-1/GIP/Gcg receptor triple agonist as described herein may be used for the following medical treatments:
(i) prevention and/or treatment of eating disorders, such as obesity, e.g. by decreasing food intake, increasing energy expenditure, reducing body weight, suppressing appetite, inducing satiety; treating or preventing binge-eating disorder, bulimia nervosa, and/or obesity induced by administration of an antipsychotic or a steroid; reduction of gastric motility; delaying gastric emptying; increasing physical mobility; and/or prevention and/or treatment of comorbidities to obesity, such as osteoarthritis and/or urine incontinence;
(ii) weight maintenance after successful weight loss (either drug induced or by diet and exercise)—i.e. prevention of weight gain after successful weight loss.
(iii) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C;
(iv) delaying or preventing diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes;
(v) prevention and/or treatment of liver disorders, such as hepatic steatosis, non-alcoholic fatty liver disease (NAFLD), non-alcoholic steatohepatitis (NASH), liver inflammation or fatty liver.

In some embodiment, the triple agonist as described herein is for use in prevention and/or treatment of obesity. In some embodiments, the triple agonist as described herein is for use in prevention and/or treatment of type 2 diabetes. In some embodiments, the triple agonist as described herein is for use in prevention and/or treatment of non-alcoholic steatohepatitis (NASH).

In some embodiments, the triple agonist as described herein relates to a method of prevention and/or treatment of obesity. In some embodiments, the triple agonist as described herein relates to a method of prevention and/or treatment of type 2 diabetes. In some embodiments, the triple agonist as described herein relates to a method of prevention and/or treatment of non-alcoholic steatohepatitis (NASH).

In some embodiments, the triple agonist as described herein relates to a method for weight management. In some embodiments, the triple agonist as described herein relates to a method for reduction of appetite. In some embodiments, the triple agonist as described herein relates to a method for reduction of food intake. In some embodiments, the triple agonist as described herein relates to a method of preventing or treating overweight in a subject.

### PARTICULAR EMBODIMENTS

The invention is further described by the following non-limiting embodiments:
1. A GLP-1/GIP/Gcg receptor triple agonist comprising a peptide and a substituent,
   wherein the amino acid sequence of the peptide is:
X₁X₂X₃GTFTSDYSX₁₂X₁₃LX₁₅KX₁₇X₁₈AX₂₀X₂₁FVX₂₄WLLX₂₈GGPSSGAPPPS (SEQ ID NO: 45), wherein
   X₁ is H or Y
   X₂ is Aib
   X₃ is H or Q
   X₁₂ is K, I or Y
   X₁₃ is Y or L
   X₁₅ is D or E
   X₁₇ is Q, R, or K
   X₁₈ is A, Y or K
   X₂₀ is Aib, Q, E, R, or H
   X₂₁ is E or K
   X₂₄ is N or Q
   X₂₈ is A or E;
   wherein the peptide is a C-terminal acid or amide; and
   wherein the substituent is covalently attached to the epsilon-amino group of the K in
   a position selected from the group consisting of positions 16, 17, and 21;
   or pharmaceutically acceptable salts thereof.
2. The GLP-1/GIP/Gcg receptor triple agonist according to embodiment 1,
   wherein the amino acid sequence of the peptide is:
X₁X₂X₃GTFTSDYSX₁₂X₁₃LX₁₅KX₁₇X₁₈AX₂₀X₂₁FVX₂₄WLLX₂₈GGPSSGAPPPS (SEQ ID NO: 46), wherein
   X₁ is H or Y
   X₂ is Aib
   X₃ is H or Q
   X₁₂ is I or Y
   X₁₃ is Y or L
   X₁₅ is D or E
   X₁₇ is Q, R, or K
   X₁₈ is A, Y or K
   X₂₀ is Aib, Q, E, R, or H
   X₂₁ is E or K
   X₂₄ is N or Q
   X₂₈ is A or E;
   wherein the peptide is a C-terminal acid or amide; and
   wherein a substituent is covalently attached to the epsilon amino group of the of the K in one of positions selected from the group consisting of positions 16, 17, and 21;
   or pharmaceutically acceptable salts thereof.
3. The GLP-1/GIP/Gcg receptor triple agonist according to any one of the preceding embodiments, wherein X₁X₂X₃ are selected from the group consisting of HAibH and YAibQ.
4. The GLP-1/GIP/Gcg receptor triple agonist according to any one of the preceding embodiments, wherein X₁X₂X₃ is HAibH.
5. The GLP-1/GIP/Gcg receptor triple agonist according to any one of the preceding embodiments, wherein X₁X₂X₃ is YAibQ.
6. The GLP-1/GIP/Gcg receptor triple agonist according to any one of the preceding embodiments, wherein X₁₂ is I.
7. The GLP-1/GIP/Gcg receptor triple agonist according to any one of embodiments 1-5 wherein X₁₂ is Y.
8. The GLP-1/GIP/Gcg receptor triple agonist according to any one of the preceding embodiments, wherein X₁₃ is Y.
9. The GLP-1/GIP/Gcg receptor triple agonist according to any one of embodiments 1-7 wherein X₁₃ is L.
10. The GLP-1/GIP/Gcg receptor triple agonist according to any one of the preceding embodiments, wherein X₁₅ is D.
11. The GLP-1/GIP/Gcg receptor triple agonist according to any one of embodiments 1-9 wherein X₁₅ is E.
12. The GLP-1/GIP/Gcg receptor triple agonist according to any one of the preceding embodiments, wherein SX₁₂X₁₃LX₁₅ are selected from the group consisting of SYYLE, SILLE, SYLLE, SIYLE, and SIYLD.
13. The GLP-1/GIP/Gcg receptor triple agonist according to any one of the preceding embodiments, wherein SX₁₂X₁₃LX₁₅ are selected from the group consisting of SYYLE, SILLE, SIYLE, and SIYLD.
14. The GLP-1/GIP/Gcg receptor triple agonist according to any one of embodiments 1-9 or 11-13, wherein SX₁₂X₁₃LX₁₅ are selected from the group consisting of SYYLE and SILLE.
15. The GLP-1/GIP/Gcg receptor triple agonist according to any one of the preceding embodiments, wherein X₁₇ is K.
16. The GLP-1/GIP/Gcg receptor triple agonist according to any one of embodiments 1-14, wherein X₁₇ is Q.
17. The GLP-1/GIP/Gcg receptor triple agonist according to any one of embodiments 1-14, wherein X₁₇ is R.
18. The GLP-1/GIP/Gcg receptor triple agonist according to any one of the preceding embodiments, wherein X₁₈ is A.
19. The GLP-1/GIP/Gcg receptor triple agonist according to any one of embodiments 1-17, wherein X₁₈ is Y.
20. The GLP-1/GIP/Gcg receptor triple agonist according to any one of embodiments 1-17, wherein X₁₈ is K.
21. The GLP-1/GIP/Gcg receptor triple agonist according to any one of the preceding embodiments, wherein X₂₀ is Aib.
22. The GLP-1/GIP/Gcg receptor triple agonist according to any one of embodiments 1-20, wherein X₂₀ is Q.
23. The GLP-1/GIP/Gcg receptor triple agonist according to any one of embodiments 1-20, wherein X₂₀ is E.
24. The GLP-1/GIP/Gcg receptor triple agonist according to any one of embodiments 1-20, wherein X₂₀ is R.
25. The GLP-1/GIP/Gcg receptor triple agonist according to any one of embodiments 1-20, wherein X₂₀ is H.
26. The GLP-1/GIP/Gcg receptor triple agonist according to any one of embodiments 1-16, 18-19, or 21, wherein KX₁₇X₁₃AX₂₀ are selected from the group consisting of KQAAAib, KKAAAib, KQYAAib, and KKYAAib.
27. The GLP-1/GIP/Gcg receptor triple agonist according to any one of the preceding embodiments, wherein X₂₁ is E.
28. The GLP-1/GIP/Gcg receptor triple agonist according to any one of embodiments 1-26, wherein X₂₁ is K.
29. The GLP-1/GIP/Gcg receptor triple agonist according to any one of the preceding embodiments, wherein X₂₄ is Q.
30. The GLP-1/GIP/Gcg receptor triple agonist according to any one of embodiments 1-28, wherein X₂₄ is N.
31. The GLP-1/GIP/Gcg receptor triple agonist according to any one of the preceding embodiments, wherein X₂₈ is E.
32. The GLP-1/GIP/Gcg receptor triple agonist according to any one of embodiments 1-30, wherein X₂₈ is A.
33. The GLP-1/GIP/Gcg receptor triple agonist according to any one of embodiments 1-4 6-16, 18-19, 21, 26-27, or 29-32, wherein the amino acid sequence of the peptide is: HX₂HGTFTSDYSX₁₂X₁₃LX₁₅KX₁₇X₁₈AX₂₀EFVX₂₄WLLX₂₈GGPSSGAPPPS (SEQ ID NO: 47), wherein
   X₂ is Aib
   X₁₂ is I or Y
   X₁₃ is Y or L
   X₁₅ is D or E
   X₁₇ is Q or K
   X₁₈ is A or Y
   X₂₀ is Aib
   X₂₄ is N or Q
   X₂₈ is A or E.
34. The GLP-1/GIP/Gcg receptor triple agonist according to any one of embodiments 1-4 6-9, 11-16, 18-19, 21, 26-27, or 29-33, wherein the amino acid sequence of the peptide is:
   HX₂HGTFTSDYSX₁₂X₁₃LEKX₁₇X₁₈AX₂₀EFVX₂₄WLLX₂₈GGPSSGAPPPS (SEQ ID NO: 48), wherein
   X₂ is Aib
   X₁₂ is I or Y
   X₁₃ is Y or L
   X₁₇ is Q or K
   X₁₈ is A or Y
   X₂₀ is Aib
   X₂₄ is N or Q
   X₂₈ is A or E.
35. The GLP-1/GIP/Gcg receptor triple agonist according to any one of embodiments 1-4 6-9, 11-16, 18-19, 21, 26-27, 29, 31 or 33-34, wherein the amino acid sequence of the peptide is:
   HX₂HGTFTSDYSX₁₂X₁₃LEKX₁₇X₁₈AX₂₀EFVQWLLEGGPSSGAPPPS (SEQ ID NO: 49), wherein
   X₂ is Aib
   X₁₂ is I or Y
   X₁₃ is Y or L
   X₁₇ is Q or K
   X₁₈ is A or Y
   X₂₀ is Aib.
36. The GLP-1/GIP/Gcg receptor triple agonist according to any one of embodiments 1-3 5-16, 18-19, 21, 26-27, or 29-32, wherein the amino acid sequence of the peptide is:
   YX₂QGTFTSDYSX₁₂X₁₃LX₁₅KX₁₇X₁₈AX₂₀EFVX₂₄WLLX₂₈GGPSSGAPPPS (SEQ ID NO: 50), wherein
   X₂ is Aib
   X₁₂ is I or Y
   X₁₃ is Y or L
   X₁₅ is D or E
   X₁₇ is Q or K
   X₁₈ is A or Y
   X₂₀ is Aib
   X₂₄ is N or Q
   X₂₈ is A or E.
37. The GLP-1/GIP/Gcg receptor triple agonist according to any one of embodiments 1-3, 5-9, 11-16, 18, 21, 26-27, 29, 31 or 36, wherein the amino acid sequence of the peptide is:

(SEQ ID NO: 51)
YX₂QGTFTSDYSX₁₂X₁₃LEKX₁₇AAX₂₀EFVQWLLEGGPSSGAPPPS,

wherein
X₂ is Aib
X₁₂ is I or Y
X₁₃ is Y or L
X₁₇ is Q or K
X₂₀ is Aib.
38. The GLP-1/GIP/Gcg receptor triple agonist according to any one of embodiments 1-3, 5-10, 12-13, 15-16, 18, 21, 26-27, 30, 32, or 36, wherein the amino acid sequence of the peptide is:

(SEQ ID NO: 52)
YX₂QGTFTSDYSX₁₂X₁₃LDKX₁₇AAX₂₀EFVNWLLAGGPSSGAPPPS,

wherein
X₂ is Aib
X₁₂ is I or Y
X₁₃ is Y or L
X₁₇ is Q or K
X₂₀ is Aib.
39. The GLP-1/GIP/Gcg receptor triple agonist according to any one of the preceding embodiments, wherein the peptide is a C-terminal amide.
40. The GLP-1/GIP/Gcg receptor triple agonist according to any one of embodiments 1-38, wherein the peptide is a C-terminal acid.
41. The GLP-1/GIP/Gcg receptor triple agonist according to any one of the preceding embodiments, wherein the substituent is A-B-C-, wherein
A- is Chem 1

HOOC—(CH₂)ₚ—CO—* Chem. 1:
p is an integer in the range of 16-20;
B- is selected from Chem. 2 or Chem. 3:

*—NH—CH(COOH)—(CH₂)₂—CO—* Chem. 2:

*—NH—CH₂—(CH₁₀)—CO—NH—CH(COOH)—(CH₂)₂—CO—*; Chem. 3:
C- is selected from Chem. 4 or Chem. 5:

*—[NH—((CH₂)₂—O)₂—CH₂—CO]₂—* Chem. 4:

*—[NH—(CH₂)₄—CH(NH₂)—CO]₂—* Chem. 5:
wherein * denotes the points of attachment and A-, B-, and C- are interconnected by amide bonds in the sequence indicated.
42. The GLP-1/GIP/Gcg receptor triple agonist according to embodiment 41, wherein A- is Chem. 1 and p is 16 or 18.
43. The GLP-1/GIP/Gcg receptor triple agonist according to any one of embodiments 41-42, wherein B- is Chem. 2.
44. The GLP-1/GIP/Gcg receptor triple agonist according to any one of embodiments 41-42, wherein B- is Chem. 3.
45. The GLP-1/GIP/Gcg receptor triple agonist according to any one of embodiments 41-42 or 44, wherein B- is Chem. 3a or Chem. 3b.
46. The GLP-1/GIP/Gcg receptor triple agonist according to any one of embodiments 41-45, wherein C- is Chem. 4.
47. The GLP-1/GIP/Gcg receptor triple agonist according to any one of embodiments 41-45, wherein C- is Chem. 5.
48. The GLP-1/GIP/Gcg receptor triple agonist according to any one of embodiments 41-43 or 46, wherein A- is Chem. 1 and p is 18; B- is Chem. 2; and C- is Chem. 4.
49. The GLP-1/GIP/Gcg receptor triple agonist according to any one of embodiments 41-43 or 47, wherein A- is Chem. 1 and p is 18; B- is Chem. 2; and C- is Chem. 5.
50. The GLP-1/GIP/Gcg receptor triple agonist according to any one of the preceding embodiments, wherein the substituent A-B-C- is selected from the group consisting of Chem. 6, Chem. 7, Chem. 8, Chem. 9, Chem. 10, Chem. 11, Chem. 12, and Chem. 13.
51. The GLP-1/GIP/Gcg receptor triple agonist according to any one of the preceding embodiments, wherein the substituent A-B-C- is selected from the group consisting of Chem. 12 and Chem. 13.
52. The GLP-1/GIP/Gcg receptor triple agonist according to any one of the preceding embodiments, wherein the substituent is covalently attached to the epsilon-amino group of K in position 16.
53. The GLP-1/GIP/Gcg receptor triple agonist according to any one of embodiments 1-15 or 18-51, wherein X₁₇ is K and the substituent is covalently attached to the epsilon-amino group of said K in position 17.
54. The GLP-1/GIP/Gcg receptor triple agonist according to any one of embodiments 1-26, 28-32, or 39-51, wherein X₂₁ is K and the substituent is covalently attached to the epsilon-amino group of said K in position 21.
55. The GLP-1/GIP/Gcg receptor triple agonist according to any one of the preceding embodiments, wherein triple agonist is selected from the group consisting of Compound Nos. 1-57.
56. The GLP-1/GIP/Gcg receptor triple agonist according to any one of embodiments 1-52 or 55, wherein the triple agonist is selected from the group consisting of Compound Nos. 1, 5-6, and 12-57.
57. The GLP-1/GIP/Gcg receptor triple agonist according to any one of embodiments 1-51, 53, or 55 wherein triple agonist is selected from the group consisting of Compound Nos. 2, and 9-10.
58. The GLP-1/GIP/Gcg receptor triple agonist according to any one of embodiments 1-51, or 54-55 wherein triple agonist is selected from the group consisting of Compound Nos. 3-4, 7-8, and 11.
59. The GLP-1/GIP/Gcg receptor triple agonist according to any one of embodiments 1-52 or 55-56, wherein triple agonist is Compound No. 26.
60. The GLP-1/GIP/Gcg receptor triple agonist according to any one of embodiments 1-52 or 55-56, wherein triple agonist is Compound No. 43.
61. The GLP-1/GIP/Gcg receptor triple agonist according to any one of embodiments 1-52 or 55-56, wherein triple agonist is Compound No. 44.
62. The GLP-1/GIP/Gcg receptor triple agonist according to any one of the preceding embodiments, wherein triple agonist is an agonist at hGLP-1R, hGIPR, and hGcgR.
63. The GLP-1/GIP/glucagon receptor triple agonist according to any one of the preceding embodiments, wherein triple agonist is capable of activating hGLP-1R, hGIPR, and hGcgR.
64. The GLP-1/GIP/Gcg receptor triple agonist according to any one of the preceding embodiments, wherein triple agonist is capable of activating hGLP-1R, hGIPR, and hGcgR in assay with whole cells expressing each of the three receptors.
65. The GLP-1/GIP/Gcg receptor triple agonist according to any one of the preceding embodiments, wherein triple agonist is capable of activating hGLP-1R, hGIPR, and hGcgR in vitro in a CRE luciferase assay, such as Example 2 described herein.
66. The GLP-1/GIP/Gcg receptor triple agonist according to any one of the preceding embodiments which is an agonist at hGLP-1R with an EC₅₀ of no more than 50 pM, such as no more than 10 pM, when measured in vitro without HSA, such as determined in Example 2 described herein.
67. The GLP-1/GIP/Gcg receptor triple agonist according to any one of the preceding embodiments which is an agonist at hGIPR with an EC₅₀ of no more than 50 pM, such as no more than 20 pM, when measured in vitro without HSA, such as determined in Example 2 described herein.
68. The GLP-1/GIP/Gcg receptor triple agonist according to any one of the preceding embodiments which is an agonist at hGcgR with an EC₅₀ of no more than 500 pM, such as no more than 250 pM, such as no more than 50 pM, when measured in vitro without HSA, such as determined in Example 2 described herein.
69. The GLP-1/GIP/Gcg receptor triple agonist according to any one of the preceding embodiments which is an agonist at all of hGLP-1R, hGIPR, and hGcgR with an EC₅₀ of no more than 500 pM, such as no more than 100 pM when measured in vitro without HSA, such as determined in Example 2 described herein.
70. The GLP-1/GIP/Gcg receptor triple agonist according to any one of the preceding embodiments which has improved pharmacokinetic properties.
71. The GLP-1/GIP/Gcg receptor triple agonist according to any one of the preceding embodiments which has an increased half-life.
72. The GLP-1/GIP/Gcg receptor triple agonist according to any one of the preceding embodiments which has an improved half-life when measured in minipigs, such as determined in Example 3 described herein.
73. The GLP-1/GIP/Gcg receptor triple agonist according to any one of the preceding embodiments which has a half-life in minipigs of at least 30 hours, such as at least 40 hours, or such as at least 60 hours, as determined in Example 3 described herein.
74. The GLP-1/GIP/Gcg receptor triple agonist according to any one of the preceding embodiments which has the effect in vivo of reducing food intake as determined in DIO mice, such as in Example 4 described herein.
75. The GLP-1/GIP/Gcg receptor triple agonist according to any one of the preceding embodiments which has the effect in vivo of inducing body weight loss as determined in DIO mice, such as in Example 4 described herein.
76. The GLP-1/GIP/Gcg receptor triple agonist according to any one of the preceding embodiments which has the effect in vivo of improving glucose tolerance as determined in DIO mice, such as in Example 4 described herein.
77. A pharmaceutical composition comprising the triple agonist according to any one of the preceding embodiments.
78. The pharmaceutical composition according to embodiment 77 and optionally at least one pharmaceutically acceptable excipient.
79. The pharmaceutical composition according to embodiments 77-78, wherein the triple agonist is selected from the group consisting of Compounds Nos. 1-57.
80. The pharmaceutical composition according to embodiment 77-79, wherein the triple agonist is Compound No. 26.
81. The pharmaceutical composition according to embodiment 77-79, wherein the triple agonist is Compound No. 43.
82. The pharmaceutical composition according to embodiment 77-79, wherein the triple agonist is Compound No. 44.
83. The pharmaceutical composition according to any one of embodiments 77-82 for use as a medicament.
84. The GLP-1/GIP/Gcg receptor triple agonist according to any one of embodiments 1-76 for use as a medicament.
85. The GLP-1/GIP/Gcg receptor triple agonist according to any one of embodiments 1-76 for use in the treatment of:
(i) prevention and/or treatment of eating disorders, such as obesity, e.g. by decreasing food intake, increasing energy expenditure, reducing body weight, suppressing appetite, inducing satiety; treating or preventing binge-eating disorder, bulimia nervosa, and/or obesity induced by administration of an antipsychotic or a steroid; reduction of gastric motility; delaying gastric emptying; increasing physical mobility; and/or prevention and/or treatment of comorbidities to obesity, such as osteoarthritis and/or urine incontinence;
(ii) weight maintenance after successful weight loss (either drug induced or by diet and exercise)—i.e. prevention of weight gain after successful weight loss.
(iii) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C;
(iv) delaying or preventing diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes;
(v) prevention and/or treatment of liver disorders, such as hepatic steatosis, non-alcoholic fatty liver disease (NAFLD), non-alcoholic steatohepatitis (NASH), liver inflammation or fatty liver.
86. The GLP-1/GIP/Gcg receptor triple agonist according to any one of embodiments 1-76 for use in prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C.
87. The GLP-1/GIP/Gcg receptor triple agonist according to any one of embodiments 1-76 for use in prevention and/or treatment of eating disorders, such as obesity, e.g. by decreasing food intake, increasing energy expenditure, reducing body weight, suppressing appetite, inducing satiety; treating or preventing binge-eating disorder, bulimia nervosa, and/or obesity induced by administration of an antipsychotic or a steroid; reduction of gastric motility; delaying gastric emptying; increasing physical mobility; and/or prevention and/or treatment of comorbidities to obesity, such as osteoarthritis and/or urine incontinence.
88. The GLP-1/GIP/Gcg receptor triple agonist according to any one of embodiments 1-76 for use in weight maintenance after successful weight loss (either drug induced or by diet and exercise)—i.e. prevention of weight gain after successful weight loss.
89. The GLP-1/GIP/Gcg receptor triple agonist according to any one of embodiments 1-76 for use in prevention and/or treatment of liver disorders, such as hepatic steatosis, non-alcoholic fatty liver disease (NAFLD), non-alcoholic steatohepatitis (NASH), liver inflammation or fatty liver.
90. The GLP-1/GIP/Gcg receptor triple agonist according to any one of embodiments 1-76 for use in weight management, the treatment and/or prevention of obesity and obesity related disorders.
91. The GLP-1/GIP/Gcg receptor triple agonist according to any one of embodiments 1-76 for use in the treatment and/or prevention of all forms of diabetes, e.g. type 2 diabetes and diabetes related disorders.
92. Use of the GLP-1/GIP/Gcg receptor triple agonist according to any one of embodiments 1-76 for the manufacture of a medicament for
(i) prevention and/or treatment of eating disorders, such as obesity, e.g. by decreasing food intake, increasing energy expenditure, reducing body weight, suppressing appetite, inducing satiety; treating or preventing binge-eating disorder, bulimia nervosa, and/or obesity induced by administration of an antipsychotic or a steroid; reduction of gastric motility; delaying gastric emptying; increasing physical mobility; and/or prevention and/or treatment of comorbidities to obesity, such as osteoarthritis and/or urine incontinence;
(ii) weight maintenance after successful weight loss (either drug induced or by diet and exercise)—i.e. prevention of weight gain after successful weight loss.
(iii) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C;
(iv) delaying or preventing diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes;
(v) prevention and/or treatment of liver disorders, such as hepatic steatosis, non-alcoholic fatty liver disease (NAFLD), non-alcoholic steatohepatitis (NASH), liver inflammation or fatty liver.
93. Use of the GLP-1/GIP/Gcg receptor triple agonist according to any one of embodiments 1-76 for the manufacture of a medicament for prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C.
94. Use of the GLP-1/GIP/Gcg receptor triple agonist according to any one of embodiments 1-76 for the manufacture of a medicament for prevention and/or treatment of eating disorders, such as obesity, e.g. by decreasing food intake, increasing energy expenditure, reducing body weight, suppressing appetite, inducing satiety; treating or preventing binge-eating disorder, bulimia nervosa, and/or obesity induced by administration of an antipsychotic or a steroid; reduction of gastric motility; delaying gastric emptying; increasing physical mobility; and/or prevention and/or treatment of comorbidities to obesity, such as osteoarthritis and/or urine incontinence.
95. Use of the GLP-1/GIP/Gcg receptor triple agonist according to any one of embodiments 1-76 for the manufacture of a medicament for weight maintenance after successful weight loss (either drug induced or by diet and exercise)—i.e. prevention of weight gain after successful weight loss.
96. Use of the GLP-1/GIP/Gcg receptor triple agonist according to any one of embodiments 1-76 for the manufacture of a medicament for prevention and/or treatment of liver disorders, such as hepatic steatosis, non-alcoholic fatty liver disease (NAFLD), non-alcoholic steatohepatitis (NASH), liver inflammation or fatty liver.
97. Use of the GLP-1/GIP/Gcg receptor triple agonist according to any one of embodiments 1-76 for the manufacture of a medicament for treatment and/or prevention of weight management, obesity and obesity related disorders.
98. Use of the GLP-1/GIP/Gcg receptor triple agonist according to any one of embodiments 1-76 for the manufacture of a medicament for treatment and/or prevention of all forms of diabetes, e.g. type 2 diabetes and diabetes related disorders.
99. A method of prevention and/or treatment of eating disorders, such as obesity, e.g. by decreasing food intake, increasing energy expenditure, reducing body weight, suppressing appetite, inducing satiety; treating or preventing binge-eating disorder, bulimia nervosa, and/or obesity induced by administration of an antipsychotic or a steroid; reduction of gastric motility; delaying gastric emptying; increasing physical mobility; and/or prevention and/or treatment of comorbidities to obesity, such as osteoarthritis and/or urine incontinence comprising administering a pharmaceutically active amount of the GLP-1/GIP/Gcg receptor triple agonist according to any one of embodiments 1-76.
100. A method of maintaining weight after successful weight loss (either drug induced or by diet and exercise)—i.e. prevention of weight gain after successful weight loss comprising administering a pharmaceutically active amount of the GLP-1/GIP/Gcg receptor triple agonist according to any one of embodiments 1-76.
101. A method of prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C comprising administering a pharmaceutically active amount of the GLP-1/GIP/Gcg receptor triple agonist according to any one of embodiments 1-76.
102. A method of delaying or preventing diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes comprising administering a pharmaceutically active amount of the GLP-1/GIP/Gcg receptor triple agonist according to any one of embodiments 1-76.
103. A method of prevention and/or treatment of liver disorders, such as hepatic steatosis, non-alcoholic fatty liver disease (NAFLD), non-alcoholic steatohepatitis (NASH), liver inflammation or fatty liver comprising administering a pharmaceutically active amount of the GLP-1/GIP/Gcg receptor triple agonist according to any one of embodiments 1-76.
104. A method of treatment and/or prevention of weight management, obesity and obesity related disorders comprising administering a pharmaceutically active amount of the GLP-1/GIP/Gcg receptor triple agonist according to any one of embodiments 1-76.
105. A method of treatment and/or prevention of all forms of diabetes, e.g. type 2 diabetes and diabetes related disorders comprising administering a pharmaceutically active amount of the GLP-1/GIP/Gcg receptor triple agonist according to any one of embodiments 1-76.
106. A method for preparing the GLP-1/GIP/Gcg receptor triple agonist according to any of embodiments 1-76.

### EXAMPLES

This experimental part starts with a list of abbreviations and is followed by a section including general methods for synthesizing and characterising the triple agonists as described herein. Then follows a number of examples which relate to preparation of specific triple agonists and selected comparative compounds, and at the end a number of examples relating to the activity and properties of the triple agonists.

Examples serve to illustrate the invention.

### List of Abbreviations

The following abbreviations are used in the following, in alphabetical order:
Ac: acetyl
Ado: 8-amino-3,6-dioxaoctanoic acid
Aib: alpha-aminoisobutyric acid (or a-aminoisobutyric acid)
amu: atomic mass unit
API-ES: atmospheric pressure ionization-electrospray
AUC: area under the curve
BHK: baby hamster kidney
Boc: tert-butyloxycarbonyl
BW: body weight
CI-HOBt: 6-chloro-1-hydroxybenzotriazole
CRE: cAMP response element
DCM: dichloromethane
DIC: diisopropylcarbodiimide
DIPEA: N,N-diisopropylethylamine
DMEM: Dulbecco's Modified Eagle's Medium
DPBS: Dulbecco's phosphate buffered saline
EDTA: ethylenediaminetetraacetic acid
ELISA: enzyme linked immunosorbent assay
equiv: molar equivalent
FBS: fetal bovine serum
Fmoc: 9-fluorenylmethyloxycarbonyl
Gcg: glucagon
GcgR: glucagon receptor
GIP: glucose-dependent insulinotropic polypeptide
GIPR: glucose-dependent insulinotropic polypeptide receptor
GLP-1: glucagon-like peptide 1
GLP-1R: glucagon-like peptide 1 receptor
HEPES: 4-(2-hydroxyethyl)-1-piperazineethanesulfonic acid
HFIP: 1,1,1,3,3,3-hexafluoro-2-propanol or hexafluoroisopropanol
hGcgR: human glucagon receptor
hGIPR: human glucose-dependent insulinotropic polypeptide receptor
hGLP-1R: human glucagon-like peptide 1 receptor
HPLC: high performance liquid chromatography
i.p.: intraperitoneal
IPGTT: intraperitoneal glucose tolerance test
i.v. intravenously
LCMS: liquid chromatography mass spectroscopy
MeCN: acetonitrile
mM: millimolar
mmol: millimoles
min: minutes
Mtt: 4-methyltrityl
MW: molecular weight
NMP: 1-methyl-pyrrolidin-2-one
OtBu: tert-butyl ester
Oxyma Pure®: cyano-hydroxyimino-acetic acid ethyl ester
Pbf: 2,2,4,6,7-pentamethyldihydrobenzofuran-5-sulfonyl
PBS: phosphate buffered saline
PK: pharmacokinetic
pM: picomolar
rpm: rounds per minute
Rt: retention time
SEM: standard error of the mean
SPPS: solid phase peptide synthesis
tBu: tert-butyl
TFA: trifluoroacetic acid
TIS: triisopropylsilane
Trt: triphenylmethyl or trityl
Trx: tranexamic acid
TSTU: O—(N-succinimidyl)-1,1,3,3-tetramethyluronium tetrafluoroborate

### General Methods of Preparation

Methods for solid phase peptide synthesis (SPPS methods, including methods for de-protection of amino acids, methods for cleaving the peptide from the resin, and for its purification), as well as methods for detecting and characterising the resulting peptide (LCMS methods) are described here below.

Resins employed for the preparation of C-terminal peptide amides were H-Rink Amide-ChemMatrix resin (loading e.g. 0.5 mmol/g) or Rink Amide AM polystyrene resin (loading e.g. 0.6 mmol/g) or PAL Amide AM resin (loading e.g. 0.6 mmol/g). Resins employed for the preparation of C-terminal peptide acids were Wang-polystyrene resin pre-loaded with the appropriate C-terminal Fmoc-protected amino acid (loading e.g. 0.6 mmol/g). The Fmoc-protected amino acid derivatives used, unless specifically stated otherwise, were the standard recommended: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-Lys(Mtt)-OH, Fmoc-Aib-OH, etc. supplied from e.g. AAPPTEC, Anaspec, Bachem, ChemImpex, Iris Biotech, Midwest Biotech, Gyros Protein Technologies or Novabiochem. Where nothing else is specified, the natural L-form of the amino acids are used. The N-terminal amino acid was employed with Boc protection at the alpha-amino group (e.g. Boc-Tyr(tBu)-OH for peptides with Tyr at the N-terminus or Boc-His(Trt)-OH for peptides with His at the N-terminus).

In case of modular albumin binding moiety attachment using SPPS, the following suitably protected building blocks such as but not limited to Fmoc-8-amino-3,6-dioxaoctanoic acid (Fmoc-Ado-OH), Fmoc-tranexamic acid (Fmoc-Trx-OH), Boc-Lys(Fmoc)-OH, Fmoc-Glu-OtBu, octadecanedioic acid mono-tert-butyl ester, nonadecanedioic acid mono-tert-butyl ester, eicosanedioic acid mono-tert-butyl ester, or tetradecanedioic acid mono-tert-butyl ester were used. All operations stated below were performed within a 0.1-0.5 mmol synthesis scale range.

### 1. Synthesis of Resin-Bound Protected Peptide Backbone:

### Method: SPPS_A

SPPS was performed using Fmoc based chemistry on a Protein Technologies SymphonyX solid phase peptide synthesizer, using the manufacturer supplied protocols with minor modifications. Mixing was accomplished by occasional bubbling with nitrogen. The step-wise assembly was performed using the following steps: 1) pre-swelling of resin in DMF; 2) Fmoc-deprotection by the use of 20% (v/v) piperidine in DMF for two treatments of 10 min each; 3) washes with DMF to remove piperidine; 4) coupling of Fmoc-amino acid by the addition of Fmoc-amino acid (4-12 equiv) and Oxyma Pure® (4-12 equiv) as a 0.3-0.6 M solution each in DMF, followed by addition of DIC (4-12 equiv) as a 0.6-1.2 M solution in DMF, with an optional addition of DMF to reduce the final concentration of each component as necessary, then mixing for 0.5-4 h; 4) washes with DMF to remove excess reagents; 5) final washes with DCM at the completion of the assembly. Some amino acids such as, but not limited to, those following a sterically hindered amino acid (e.g. Aib) were coupled with an extended reaction time (e.g. 4 h) to ensure reaction completion.

### Method: SPPS_B

The protected peptidyl resin was synthesized according to the Fmoc strategy on an Applied Biosystems 431A solid-phase peptide synthesizer using the manufacturer supplied general Fmoc protocols. Mixing was accomplished by vortexing and occasional bubbling with nitrogen. The step-wise assembly was done using the following steps: 1) activation of Fmoc-amino acid by dissolution of solid Fmoc-acid acid (10 equiv) in CI-HOBt (10 equiv) as a 1 M solution in NMP, then addition of DIC (10 equiv) as a 1 M solution in NMP, then mixing simultaneous to steps 2-3; 2) Fmoc-deprotection by the use of 20% (v/v) piperidine in NMP for one treatment of 3 min then a second treatment of 15 min; 3) washes with NMP to remove piperidine; 4) addition of activated Fmoc-amino acid solution to resin, then mixing for 45-90 min; 4) washes with NMP to remove excess reagents; 5) final washes with DCM at the completion of the assembly. The standard protected amino acid derivatives listed above were supplied in pre-weighed cartridges (from e.g. Midwest Biotech), and non-standard derivatives were weighed by hand. Some amino acids such as, but not limited to, those following a sterically hindered amino acid (e.g. Aib) were “double coupled” to ensure reaction completion, meaning that after the first coupling (e.g. 45 min) the resin is drained, more reagents are added (Fmoc-amino acid, DIC, CI-HOBt), and the mixture allowed to react again (e.g. 45 min).

### 2. Attachment of Substituent to Resin-Bound Protected Peptide Backbone

### Method: SC_A

The N-epsilon-lysine protection Mtt protection group was removed by washing the resin with 30% (v/v) HFIP in DCM for two treatments of 45 min each, following by washing with DCM and DMF. Acylation was performed on a Protein Technologies SymphonyX solid phase peptide synthesizer using the protocols described in method SPPS_A using stepwise addition of building blocks, such as, but not limited to, Boc-Lys(Fmoc)-OH, Fmoc-8-amino-3,6-dioxaoctanoic acid, Fmoc-tranexamic acid, Fmoc-Glu-OtBu, octadecanedioic acid mono-tert-butyl ester, and eicosanedioic acid mono-tert-butyl ester.

### Method: SC_B

The N-epsilon-lysine protection Mtt protection group was removed by washing the resin with 30% (v/v) HFIP in DCM for two treatments of 45 min each, following by washing with DCM and DMF. Acylation was performed on an Applied Biosystems 431A solid-phase peptide synthesizer using the protocols described in method SPPS_B using stepwise addition of building blocks, such as, but not limited to, Boc-Lys(Fmoc)-OH, Fmoc-8-amino-3,6-dioxaoctanoic acid, Fmoc-tranexamic acid, Fmoc-Glu-OtBu, octadecanedioic acid mono-tert-butyl ester, and eicosanedioic acid mono-tert-butyl ester.

### Method: SC_C

The N-epsilon-lysine protection Mtt protection group was removed by washing the resin with 75:2.5:22.5 v/v/v HFIP/TIS/DCM for three treatments of 30 min each, following by washing with DCM and DMF. Separately, 2-chlorotrityl chloride resin (loading e.g. 1.1 mmol/g) was transferred to a sintered separating funnel and swollen in DCM for 30 mins, then drained and shaken with 10% DIPEA in DCM (v/v) for 10 mins. The resin then drained and immediately treated with the first Fmoc-protected building block of the substituent (1.0 equiv) and DIPEA (2 equiv) in DCM and shaken overnight at room temperature. Methanol was then added to the mixture to cap the unreacted sites of the resin. The resin was washed with DCM, DMF, and diethyl ether before drying under vacuum. The substituent was subsequently assembled in a stepwise fashion as described in method SPPS_A. The protected substituent was cleaved from the resin using 20% HFIP in DCM (v/v) for three treatments of 10 min each, and the cleavage mixtures were concentrated in vacuo and dried under vacuum to yield the protected substituent, which was used without further manipulation. The protected substituent was dissolved in dry THF at a concentration of 0.1 M before being treated with TSTU (1.5 equiv) and DIPEA (3 equiv). The mixture was stirred at room temperature for two hours, filtered, and diluted five-fold in volume with ethyl acetate. The organic extract was washed with an equal volume of 0.1 M aqueous HCl, followed by an equal volume of water. The organic layer was subsequently dried with MgSO₄, and the volatile solvents were removed in vacuo. The activated substituent (1.5 equiv) was used directly as a 0.1 M solution in DMF containing DIPEA (3 equiv) and incubated with peptidyl resin bearing a free lysine ε-amino group for 16 hours. The resin was subsequently washed with DMF and DCM.

### 3. Cleavage of Resin Bound Peptide and Purification:

Following completion of the sidechain synthesis, the peptidyl resin was washed with DCM and dried, then treated with TFA/water/TIS (95:2.5:2.5 v/v/v) for approximately 2-3 h, followed by precipitation with diethyl ether. The precipitate was washed with diethyl ether, dissolved in a suitable solvent (e.g. 2:1 water/MeCN), and let stand until all labile adducts decomposed. Purification was performed by reversed-phase preparative HPLC (e.g. Waters 2545 binary gradient module, Waters 2489 UV/Visible detector, Waters fraction collector III; or e.g. Waters Deltaprep 4000) on a suitable column containing e.g. C8- or C18-silica gel. Separation of impurities and product elution was accomplished using an increasing gradient of MeCN in water containing 0.1% TFA. Relevant fractions were checked for identity and purity by analytical LCMS. Fractions containing the pure desired product were pooled and freeze-dried to afford the peptide TFA salt as a white solid.

4. Salt Exchange from TFA to Sodium Salt:

The freeze-dried, purified peptide was dissolved to 3-20 mg/mL in an appropriate aqueous buffer such as, but not limited to, 4:1 water/MeCN, 0.2 M sodium acetate, or 50 mM HEPES buffer pH 7.4. The solution pH was adjusted with aqueous NaOH if necessary to achieve full solubility. The buffered solutions containing the peptide were salt-exchanged using a Sep-Pak C18 cartridge (0.5-5 g). The cartridge was first equilibrated with isopropanol, then MeCN, then water. The peptide solution was applied to the cartridge, and the flow through was reapplied to ensure complete retention of peptide. The cartridge was washed with water, then a buffer solution (e.g. pH 7.5) containing such as, but not limited to, NaHCO₃, NaOAc, or Na₂HPO₄. The cartridge was then washed with water, and the peptide was eluted with 50-80% (v/v) MeCN in water. The peptide-containing eluent was freeze-dried to afford the peptide sodium salt as a white solid, which was used as such.

### General Methods of Detection and Characterisation

LCMS methods:

### Method: LCMS_A

LCMS_A was performed on a setup consisting of an Agilent 1260 Infinity series HPLC system and an Agilent Technologies 6120 Quadrupole MS. Eluents were defined as: A: 0.05% (v/v) TFA in water; B: 0.05% (v/v) TFA in 9:1 (v/v) MeCN/water. The analysis was performed at a column temperature of 37° C. by injecting an appropriate volume of the sample onto the column which was eluted with a gradient of A and B. Column: Phenomenex Kinetex C8, 2.6 μm, 100 Å, 4.6×75 mm. Gradient run time: Linear 20-100% B over 10 min at a flow rate of 1.0 mL/min. Detection: diode array detector set to 214 nm. MS ionisation mode: API-ES, positive polarity. MS scan mass range: 500-2000 amu. The most abundant isotope of each m/z is reported.

### Method: LCMS_B

LCMS_B was performed on a setup consisting of a Waters Acquity H class UPLC system and a Waters Xevo G2-XS QT of MS. Eluents were defined as: A: 0.1% (v/v) formic acid in water; B: 0.1% (v/v) formic acid in MeCN; C: 0.1% (v/v) TFA in water. The analysis was performed at a column temperature of 40° C. by injecting an appropriate volume of the sample onto the column which was eluted with a gradient of A and B. Column: Waters Acquity BEH, C-18, 1.7 μm, 2.1×50 mm. Gradient run time: Linear 5-95% B over 4.0 min at a flow rate of 0.4 mL/min in the presence of a constant 5% C. MS ionisation mode: ES, positive polarity. MS scan mass range: 50-4000 amu. The most abundant isotope of each m/z is reported.

### Example 1: Synthesis of Compounds

The compounds are in the following described using single letter amino acid codes, except for Aib. The substituent is included after the lysine (K) residue to which it is attached.

### Compound No. 1

### Y-Aib-QGTFTSDYSIYLD-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-QAA-Aib-EFVNWLLAGGPSSGAPPPS-NH₂

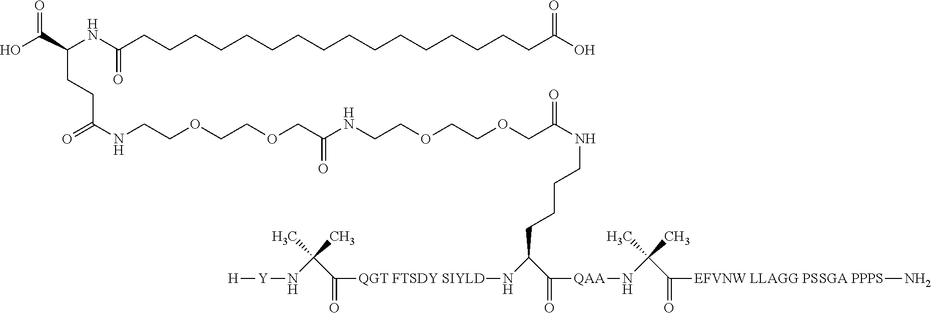

Peptide backbone: SEQ ID NO: 1, C-terminal amide
Substituent: Chem. 6 attached via ε-amino group of K16
Synthesis methods: SPPS_A; SC_A
Molecular weight (average) calculated: 4821.3 Da
LCMS_B: Rt=3.4 min; found [M+3H]³⁺ 1607.1, [M+4H]⁴⁺ 1205.6.

### Compound No. 2

### Y-Aib-QGTFTSDYSIYLDK-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-AA-Aib-EFVNWLLAGGPSSGAPPPS-NH₂

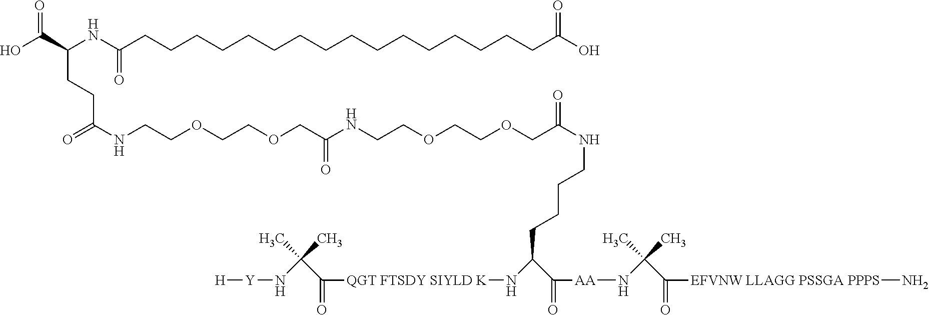

Peptide backbone: SEQ ID NO: 2, C-terminal amide
Substituent: Chem. 6 attached via ε-amino group of K17
Synthesis methods: SPPS_A; SC_A
Molecular weight (average) calculated: 4821.4 Da
LCMS_B: Rt=3.0 min; found [M+2H]²⁺ 2411.8, [M+3H]³⁺ 1607.8, [M+4H]⁴⁺ 1206.1.

### Compound No. 3

### Y-Aib-QGTFTSDYSIYLDKQAA-Aib-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-FVNWLLAGGPSSGAPPPS-NH₂

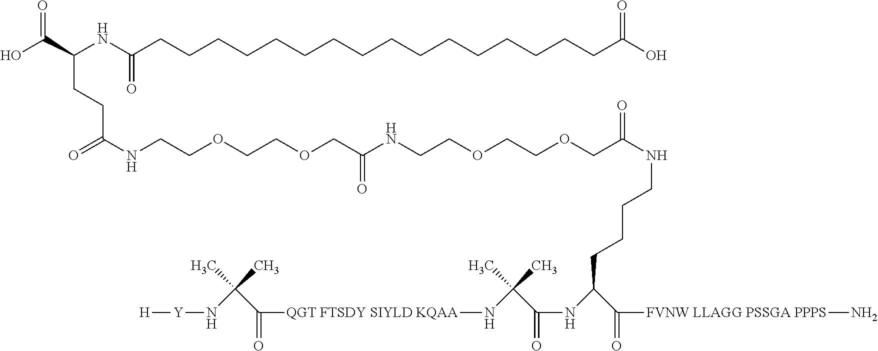

Peptide backbone: SEQ ID NO: 3, C-terminal amide
Substituent: Chem. 6 attached via ε-amino group of K21
Synthesis methods: SPPS_A; SC_A
Molecular weight (average) calculated: 4820.4 Da
LCMS_B: Rt=3.0 min; found [M+3H]³⁺ 1606.8, [M+4H]⁴⁺ 1205.6.

### Compound No. 4

### Y-Aib-QGTFTSDYSIYLDKQAA-Aib-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-FVQWLLEGGPSSGAPPPS-NH₂

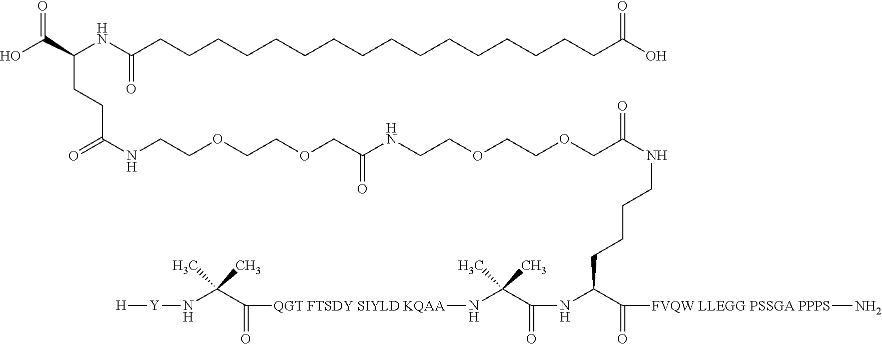

Peptide backbone: SEQ ID NO: 4, C-terminal amide
Substituent: Chem. 6 attached via ε-amino group of K21
Synthesis methods: SPPS_A; SC_A
Molecular weight (average) calculated: 4892.5 Da
LCMS_A: Rt=6.1 min; found [M+3H]³⁺ 1631.5, [M+4H]⁴⁺ 1224.0.

### Compound No. 5

### Y-Aib-QGTFTSDYSIYLD-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-QAA-Aib-EFVNWLLAGGPSSGAPPPS-NH₂

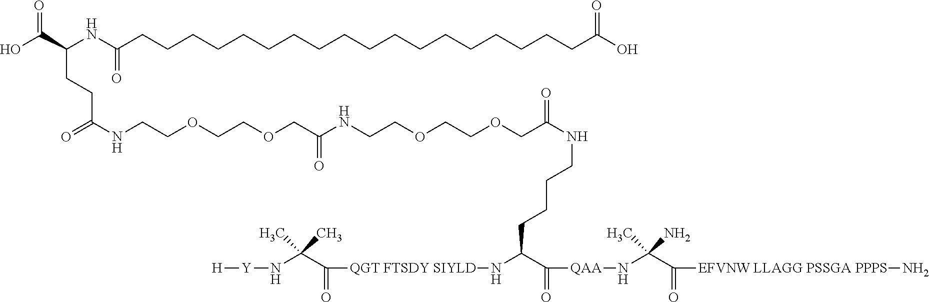

Peptide backbone: SEQ ID NO: 1, C-terminal amide
Substituent: Chem. 12 attached via ε-amino group of K16
Synthesis methods: SPPS_A; SC_C
Molecular weight (average) calculated: 4849.4 Da
LCMS_B: Rt=3.5 min; found [M+2H]²⁺ 2424.7, [M+3H]³⁺ 1616.5, [M+4H]⁴⁺ 1212.6.

### Compound No. 6

### Y-Aib-QGTFTSDYSIYLD-K[2-[2-[2-[[2-[2-[2-[[(4S)-4-carboxy-4-[[4-[(19-carboxynonadecanoylamino)methyl]cyclohexanecarbonyl]amino]butanoyl]amino]ethoxy]ethoxy]acetyl]amino]ethoxy]ethoxy]acetyl]-QAA-Aib-EFVNWLLAGGPSSGAPPPS-NH₂

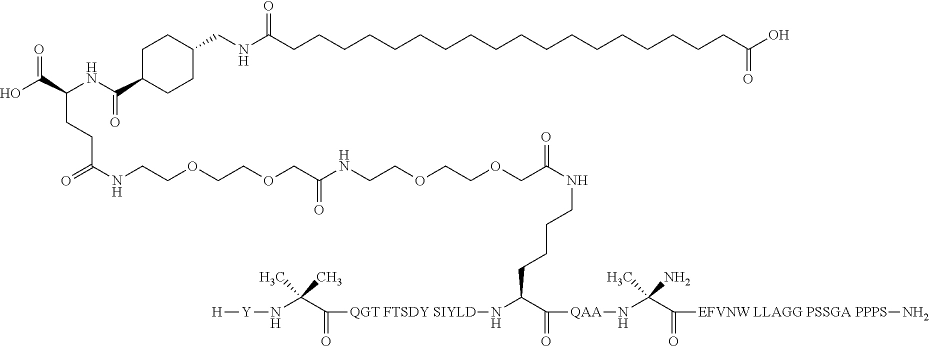

Peptide backbone: SEQ ID NO: 1, C-terminal amide
Substituent: Chem. 10 attached via ε-amino group of K16
Synthesis methods: SPPS_A; SC_C
Molecular weight (average) calculated: 4988.6 Da
LCMS_B: Rt=3.6 min; found [M+2H]²⁺ 2495.1, [M+3H]³⁺ 1663.4, [M+4H]⁴⁺ 1247.8.

### Compound No. 7

### Y-Aib-QGTFTSDYSIYLDKQAA-Aib-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-FVQWLLEGGPSSGAPPPS-NH₂

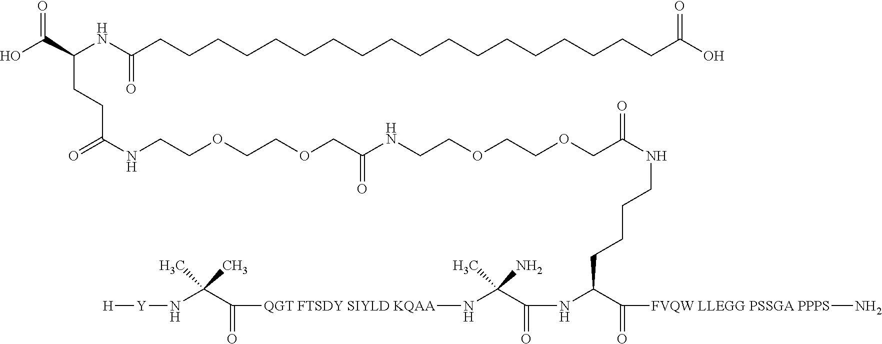

Peptide backbone: SEQ ID NO: 4, C-terminal amide
Substituent: Chem. 12 attached via ε-amino group of K21
Synthesis methods: SPPS_A; SC_C
Molecular weight (average) calculated: 4920.5 Da
LCMS_B: Rt=3.2 min; found [M+2H]²⁺ 2461.3, [M+3H]³⁺ 1640.9.

### Compound No. 8

### Y-Aib-QGTFTSDYSIYLDKQAA-Aib-K[2-[2-[2-[[2-[2-[2-[[(4S)-4-carboxy-4-[[4-[(19-carboxynonadecanoylamino)methyl]cyclohexanecarbonyl]amino]butanoyl]amino]ethoxy]ethoxy]acetyl]amino]ethoxy]ethoxy]acetyl]-FVQWLLEGGPSSGAPPPS-NH₂

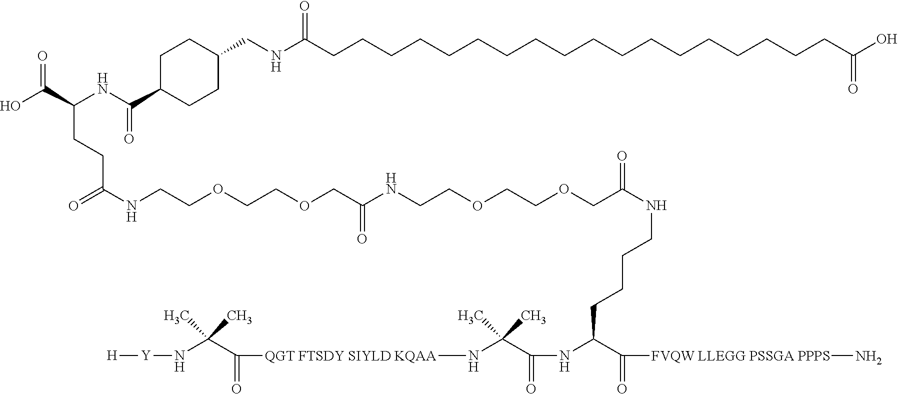

Substituent: Chem. 10 attached via ε-amino group of K21
Synthesis methods: SPPS_A; SC_C
Molecular weight (average) calculated: 5059.7 Da
LCMS_B: Rt=3.3 min; found [M+3H]³⁺ 1686.5, [M+4H]⁴⁺ 1265.4.

### Compound No. 9

### Y-Aib-QGTFTSDYSIYLDK-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl-AA-Aib-EFVNWLLAGGPSSGAPPPS-NH₂

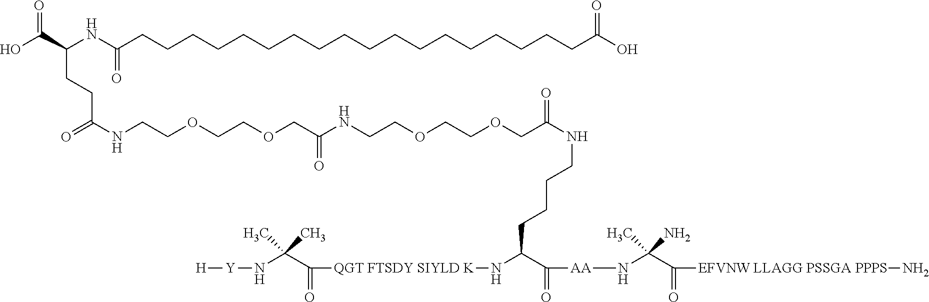

Peptide backbone: SEQ ID NO: 2, C-terminal amide
Substituent: Chem. 12 attached via ε-amino group of K17
Synthesis methods: SPPS_A; SC_C
Molecular weight (average) calculated: 4849.4 Da
LCMS_B: Rt=3.2 min; found [M+H]⁺ 4850.0 [M+2H]²⁺ 2426.1, [M+3H]³⁺ 1617.6.

### Compound No. 10

### Y-Aib-QGTFTSDYSIYLDK-K[2-[2-[2-[[2-[2-[2-[[(4S)-4-carboxy-4-[[4-[(19-carboxynonadecanoylamino)methyl]cyclohexanecarbonyl]amino]butanoyl]amino]ethoxy]ethoxy]acetyl]amino]ethoxy]ethoxy]acetyl]-AA-Aib-EFVNWLLAGGPSSGAPPPS-NH₂

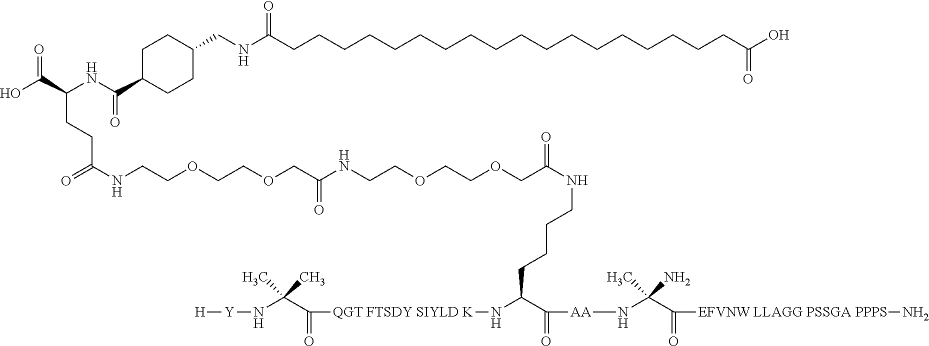

Peptide backbone: SEQ ID NO: 2, C-terminal amide
Substituent: Chem. 10 attached via ε-amino group of K17
Synthesis methods: SPPS_A; SC_C
Molecular weight (average) calculated: 4988.6 Da
LCMS_B: Rt=3.3 min; found [M+H]⁺ 4988.0, [M+2H]²⁺ 2495.4, [M+3H]³⁺ 1663.6.

### Compound 11

### Y-Aib-QGTFTSDYSIYLEKQAA-Aib-K[2-[2-[2-[[2-[2-[2-[[(4S)-4-carboxy-4-[[4-[(19-carboxynonadecanoylamino)methyl]cyclohexanecarbonyl]amino]butanoyl]amino]ethoxy]ethoxy]acetyl]amino]ethoxy]ethoxy]acetyl]-FVQWLLEGGPSSGAPPPS-NH₂

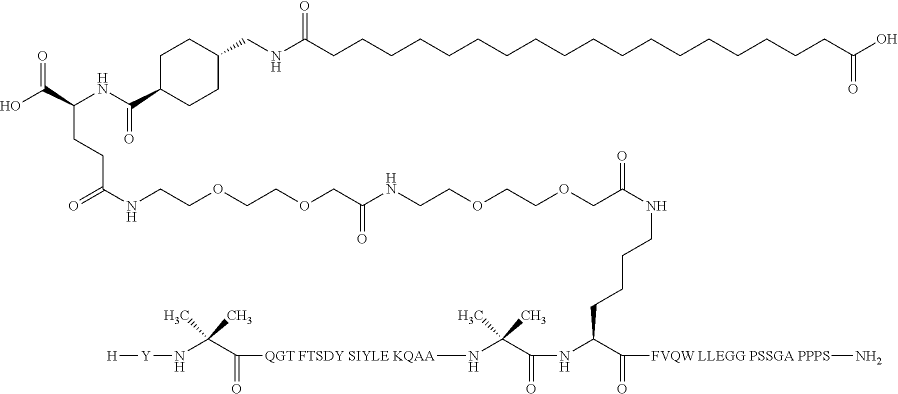

Peptide backbone: SEQ ID NO: 5, C-terminal amide
Substituent: Chem. 10 attached via ε-amino group of K21
Synthesis methods: SPPS_A; SC_A
Molecular weight (average) calculated: 5073.7 Da
LCMS_A: Rt=6.6 min; found [M+3H]³⁺ 1692.0, [M+4H]⁴⁺ 1269.4.

### Compound No. 12

### Y-Aib-QGTFTSDYSIYLE-K[2-[2-[2-[[2-[2-[2-[[(4S)-4-carboxy-4-[[4-[(19-carboxynonadecanoylamino)methyl]cyclohexanecarbonyl]amino]butanoyl]amino]ethoxy]ethoxy]acetyl]amino]ethoxy]ethoxy]acetyl]-QAA-Aib-EFVNWLLAGGPSSGAPPPS-NH₂

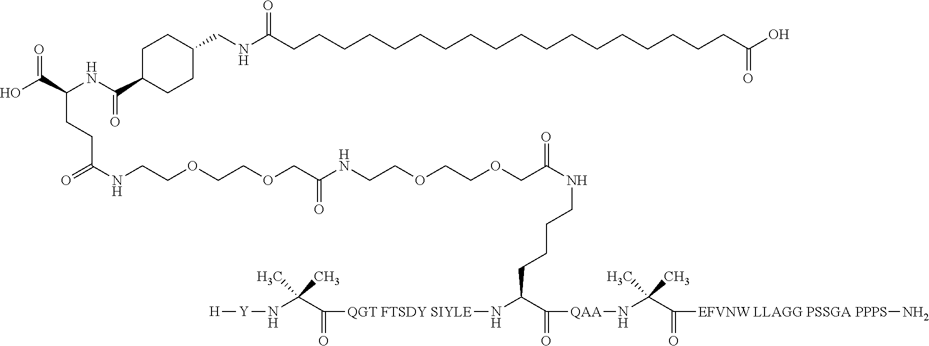

Peptide backbone: SEQ ID NO: 6, C-terminal amide
Substituent: Chem. 10 attached via ε-amino group of K16
Synthesis methods: SPPS_A; SC_B
Molecular weight (average) calculated: 5002.6 Da
LCMS_A: Rt=6.9 min; found [M+3H]³⁺ 1668.4, [M+4H]⁴⁺ 1251.5.

### Compound No. 13

### Y-Aib-QGTFTSDYSIYLE-K[(2S)-2-amino-6-[[(2S)-2-amino-6-[[(4S)-4-carboxy-4-[[4-[(19-carboxynonadecanoylamino)methyl]cyclohexanecarbonyl]amino]butanoyl]amino]hexanoyl]amino]hexanoyl]-QAA-Aib-EFVNWLLAGGPSSGAPPPS-NH₂

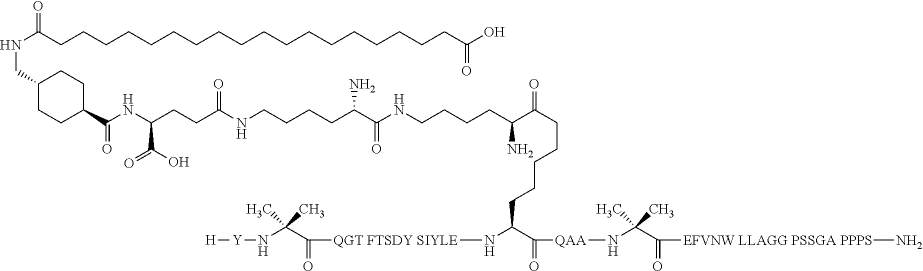

Peptide backbone: SEQ ID NO: 6, C-terminal amide
Substituent: Chem. 11 attached via ε-amino group of K16
Synthesis methods: SPPS_A; SC_B
Molecular weight (average) calculated: 4968.7 Da
LCMS_A: Rt=6.3 min; found [M+3H]³⁺ 1657.0, [M+4H]⁴⁺ 1243.1.

### Compound No. 14

### Y-Aib-QGTFTSDYSIYLE-K[2-[2-[2-[[2-[2-[2-[[(4S)-4-carboxy-4-[[4-[(19-carboxynonadecanoylamino)methyl]cyclohexanecarbonyl]amino]butanoyl]amino]ethoxy]ethoxy]acetyl]amino]ethoxy]ethoxy]acetyl]-QAA-Aib-EFVQWLLEGGPSSGAPPPS-NH₂

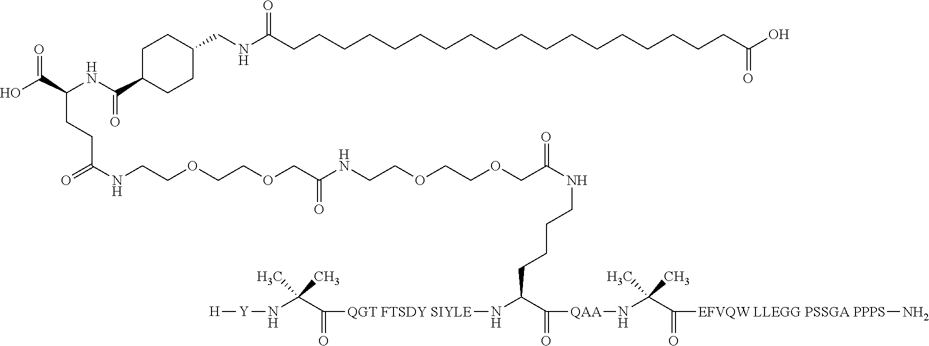

Peptide backbone: SEQ ID NO: 7, C-terminal amide
Substituent: Chem. 10 attached via ε-amino group of K16
Synthesis methods: SPPS_A; SC_B
Molecular weight (average) calculated: 5102.7 Da
LCMS_A: Rt=7.0 min; found [M+3H]³⁺ 1701.7, [M+4H]⁴⁺ 1276.4.

### Compound No. 15

### Y-Aib-QGTFTSDYSIYLE-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-QAA-Aib-EFVNWLLAGGPSSGAPPPS-NH₂

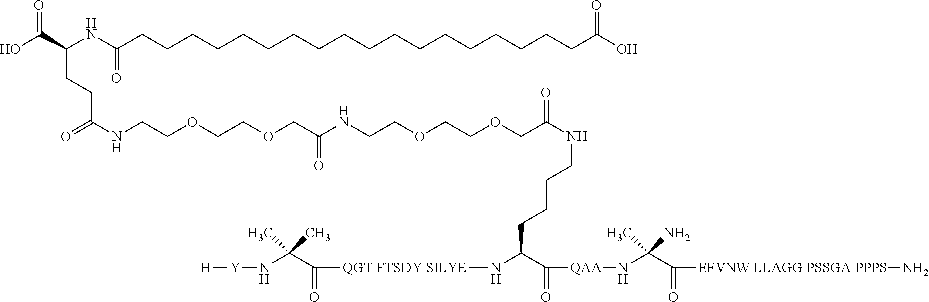

Peptide backbone: SEQ ID NO: 6, C-terminal amide
Substituent: Chem. 12 attached via ε-amino group of K16
Synthesis methods: SPPS_A; SC_A
Molecular weight (average) calculated: 4863.4 Da
LCMS_A: Rt=6.9 min; found [M+3H]³⁺ 1621.7, [M+4H]⁴⁺ 1216.7.

### Compound No. 16

### Y-Aib-QGTFTSDYSIYLE-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-QAA-Aib-EFVQWLLEGGPSSGAPPPS-NH₂

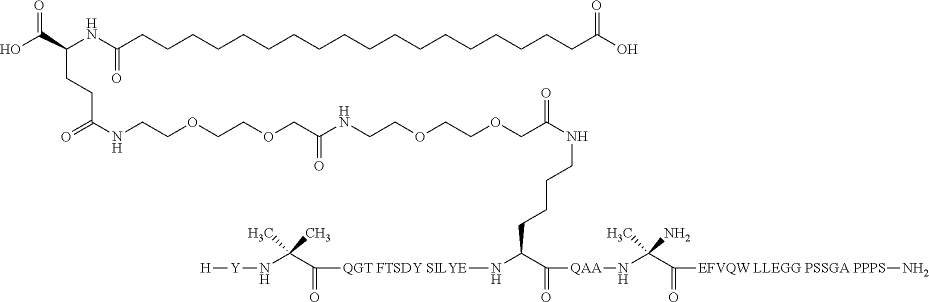

Peptide backbone: SEQ ID NO: 7, C-terminal amide
Substituent: Chem. 12 attached via ε-amino group of K16
Synthesis methods: SPPS_A; SC_A
Molecular weight (average) calculated: 4935.5 Da
LCMS_A: Rt=7.0 min; found [M+3H]³⁺ 1645.7, [M+4H]⁴⁺ 1234.6.

### Compound No. 17

### Y-Aib-QGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂

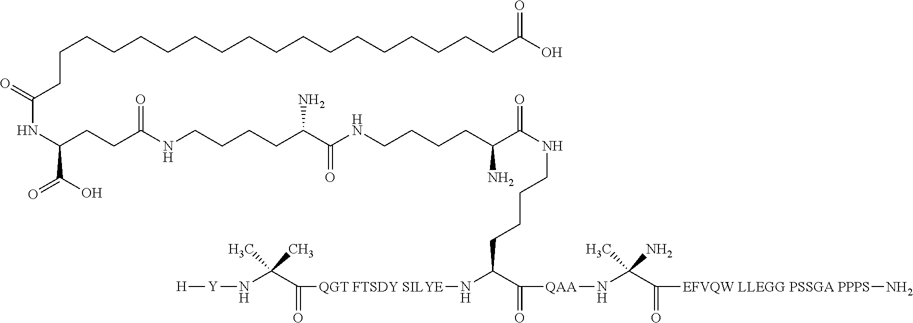

Peptide backbone: SEQ ID NO: 7, C-terminal amide
Substituent: Chem. 13 attached via ε-amino group of K16
Synthesis methods: SPPS_A; SC_A
Molecular weight (average) calculated: 4901.5 Da
LCMS_A: Rt=6.2 min; found [M+3H]³⁺ 1634.5, [M+4H]⁴⁺ 1226.7.

### Compound No. 18

### Y-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂

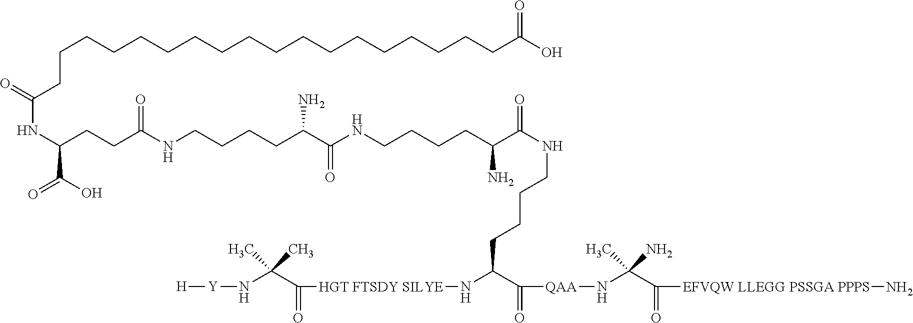

Peptide backbone: SEQ ID NO: 8, C-terminal amide
Substituent: Chem. 13 attached via ε-amino group of K16
Synthesis methods: SPPS_A; SC_A
Molecular weight (average) calculated: 4910.5 Da
LCMS_A: Rt=6.1 min; found [M+3H]³⁺ 1637.5, [M+4H]⁴⁺ 1228.4.

### Compound No. 19

### H-Aib-QGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂

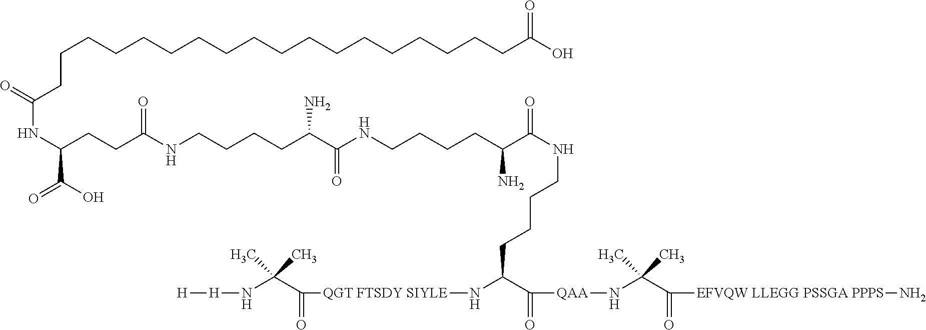

Peptide backbone: SEQ ID NO: 9, C-terminal amide
Substituent: Chem. 13 attached via ε-amino group of K16
Synthesis methods: SPPS_A; SC_A
Molecular weight (average) calculated: 4875.5 Da
LCMS_A: Rt=7.0 min; found [M+3H]³⁺ 1625.8, [M+4H]⁴⁺ 1219.7.

### Compound No. 20

### Y-Aib-QGTFTSDYSIYLE-K[2-[2-[2-[[2-[2-[2-[[(4S)-4-carboxy-4-[[4-[(19-carboxynonadecanoylamino)methyl]cyclohexanecarbonyl]amino]butanoyl]amino]ethoxy]ethoxy]acetyl]amino]ethoxy]ethoxy]acetyl]-QAAQEFVQWLLEGGPSSGAPPPS-NH₂

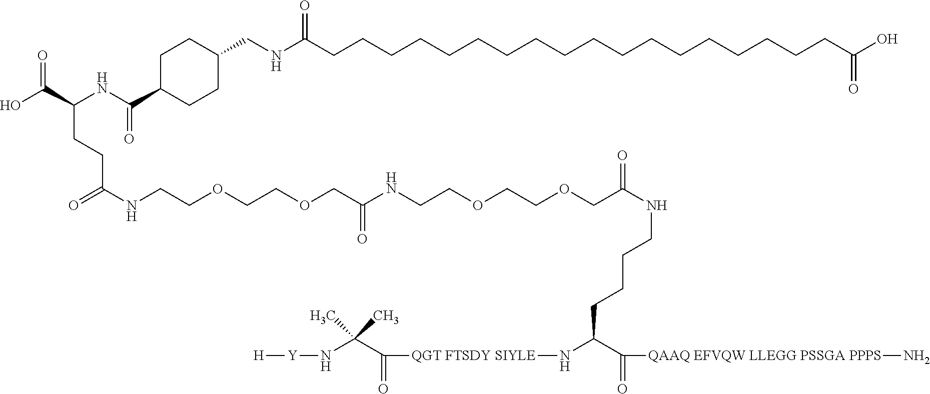

Peptide backbone: SEQ ID NO: 10, C-terminal amide
Substituent: Chem. 10 attached via ε-amino group of K16
Synthesis methods: SPPS_B; SC_B
Molecular weight (average) calculated: 5117.7 Da
LCMS_A: Rt=6.8 min; found [M+3H]³⁺ 1706.8, [M+4H]⁴⁺ 1280.3.

### Compound No. 21

### H-Aib-HGTFTSDYSIYLE-K[(2S)-2-amino-6-[[(2S)-2-amino-6-[[(4S)-4-carboxy-4-[[4-[(19-carboxynonadecanoylamino)methyl]cyclohexanecarbonyl]amino]butanoyl]amino]hexanoyl]amino]hexanoyl]-QAA-Aib-EFVQWLLEGGPSSGAPPPS-NH₂

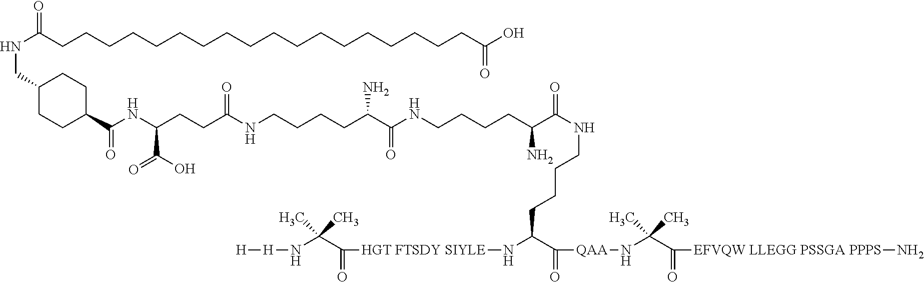

Peptide backbone: SEQ ID NO: 11, C-terminal amide
Substituent: Chem. 11 attached via ε-amino group of K16
Synthesis methods: SPPS_A; SC_B
Molecular weight (average) calculated: 5023.7 Da
LCMS_A: Rt=6.1 min; found [M+3H]³⁺ 1675.3, [M+4H]⁴⁺ 1256.7.

### Compound No. 22

### H-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂

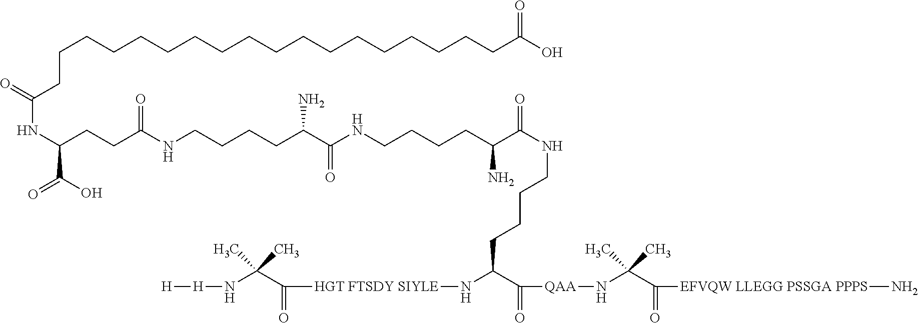

Peptide backbone: SEQ ID NO: 11, C-terminal amide
Substituent: Chem. 13 attached via ε-amino group of K16
Synthesis methods: SPPS_A; SC_B
Molecular weight (average) calculated: 4884.5 Da
LCMS_A: Rt=6.0 min; found [M+3H]³⁺ 1628.8, [M+4H]⁴⁺ 1222.0.

### Compound No. 23

### H-Aib-HGTFTSDYSIYLE-K[(2S)-2-amino-6-[[(2S)-2-amino-6-[[(4S)-4-carboxy-4-[[4-[(19-carboxynonadecanoylamino)methyl]cyclohexanecarbonyl]amino]butanoyl]amino]hexanoyl]amino]hexanoyl]-QAAQEFVQWLLEGGPSSGAPPPS-NH₂

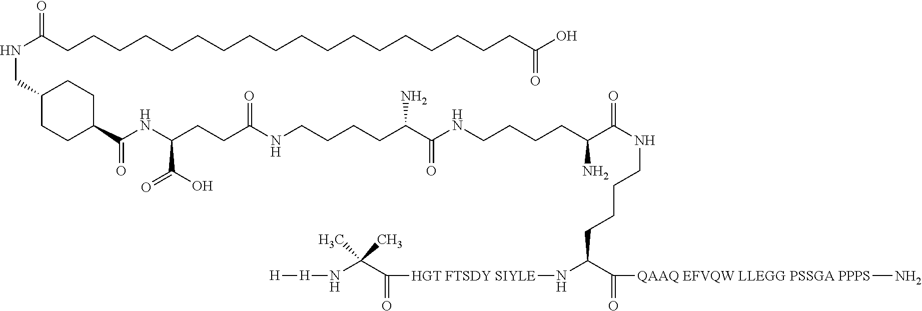

Peptide backbone: SEQ ID NO: 12, C-terminal amide
Substituent: Chem. 11 attached via ε-amino group of K16
Synthesis methods: SPPS_B; SC_B
Molecular weight (average) calculated: 5066.7 Da
LCMS_A: Rt=6.0 min; found [M+3H]³⁺ 1689.7, [M+4H]⁴⁺ 1267.5.

### Compound No. 24

### H-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QYA-Aib-EFVQWLLEGGPSSGAPPPS-NH₂

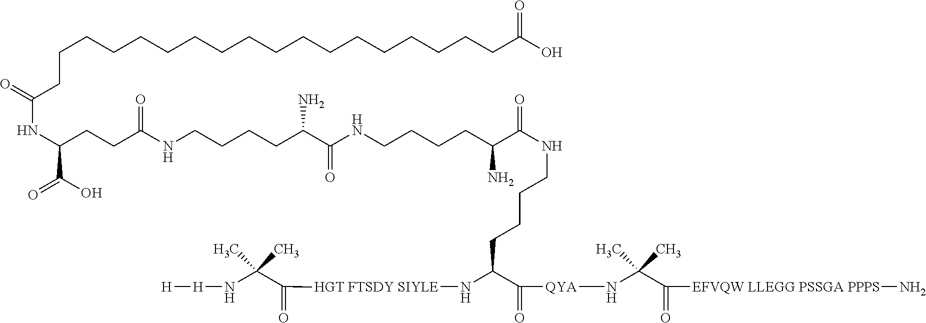

Peptide backbone: SEQ ID NO: 13, C-terminal amide
Substituent: Chem. 13 attached via ε-amino group of K16
Synthesis methods: SPPS_A; SC_B
Molecular weight (average) calculated: 4976.6 Da
LCMS_A: Rt=6.0 min; found [M+3H]³⁺ 1659.8, [M+4H]⁴⁺ 1244.9.

### Compound No. 25

### H-Aib-HGTFTSDYSYYLE-K[(2S)-2-amino-6-[[(2S)-2-amino-6-[[(4S)-4-carboxy-4-[[4-[(19-carboxynonadecanoylamino)methyl]cyclohexanecarbonyl]amino]butanoyl]amino]hexanoyl]amino]hexanoyl]-QAA-Aib-EFVQWLLEGGPSSGAPPPS-NH₂

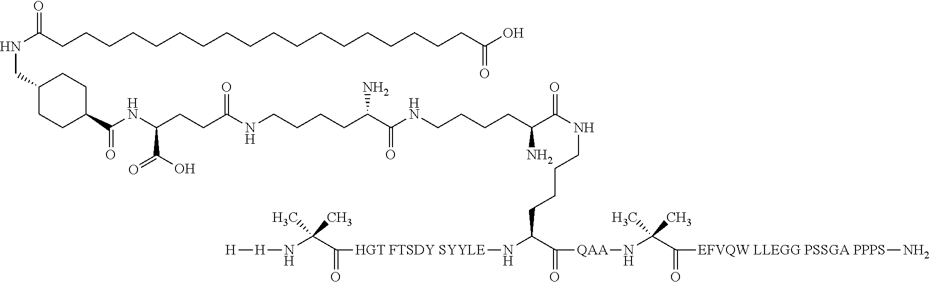

Peptide backbone: SEQ ID NO: 14, C-terminal amide
Substituent: Chem. 11 attached via ε-amino group of K16
Synthesis methods: SPPS_A; SC_B
Molecular weight (average) calculated: 5073.7 Da
LCMS_A: Rt=6.0 min; found [M+3H]³⁺ 1692.0, [M+4H]⁴⁺ 1269.1.

### Compound No. 26

### H-Aib-HGTFTSDYSYYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂

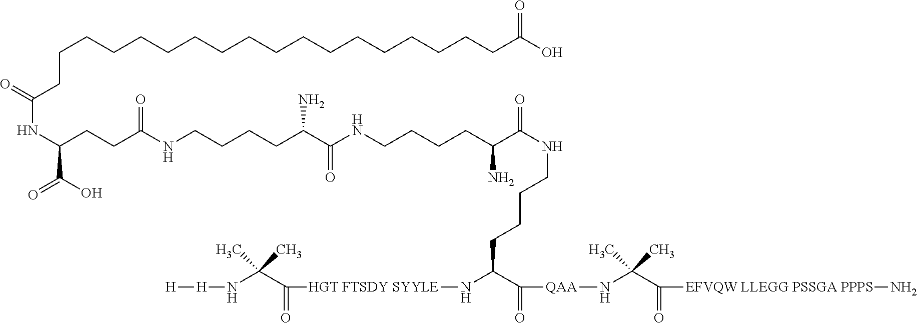

Peptide backbone: SEQ ID NO: 14, C-terminal amide
Substituent: Chem. 13 attached via ε-amino group of K16
Synthesis methods: SPPS_A; SC_B
Molecular weight (average) calculated: 4934.5 Da
LCMS_A: Rt=5.9 min; found [M+3H]³⁺ 1645.5, [M+4H]⁴⁺ 1234.5.

### Compound No. 27

### H-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAAEEFVQWLLEGGPSSGAPPPS-NH₂

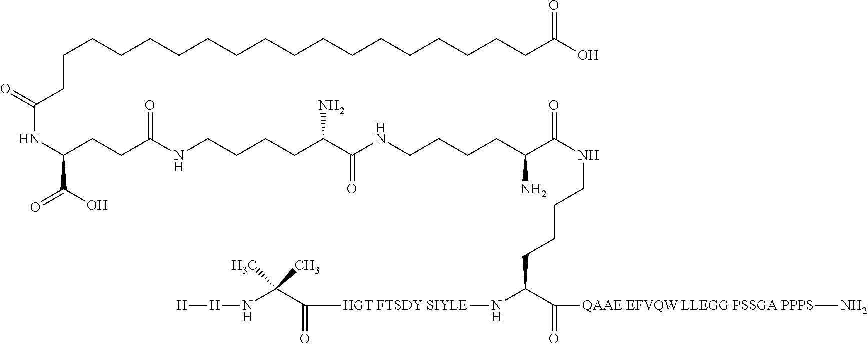

Peptide backbone: SEQ ID NO: 15, C-terminal amide
Substituent: Chem. 13 attached via ε-amino group of K16
Synthesis methods: SPPS_A; SC_B
Molecular weight (average) calculated: 4928.5 Da
LCMS_A: Rt=5.9 min; found [M+3H]³⁺ 1643.6, [M+4H]⁴⁺ 1232.9.

### Compound No. 28

### H-Aib-HGTFTSDYSIYLD-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂

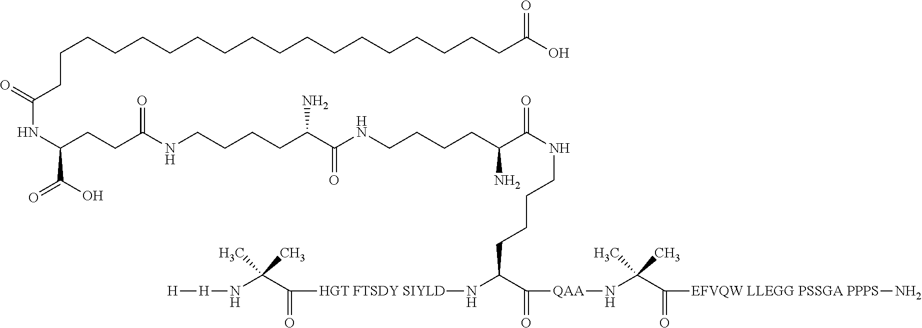

Peptide backbone: SEQ ID NO: 16, C-terminal amide
Substituent: Chem. 13 attached via ε-amino group of K16
Synthesis methods: SPPS_A; SC_B
Molecular weight (average) calculated: 4870.5 Da
LCMS_A: Rt=5.9 min; found [M+3H]³⁺ 1624.4, [M+4H]⁴⁺ 1218.5.

### Compound No. 29

### H-Aib-HGTFTSDYSYYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QYA-Aib-EFVQWLLEGGPSSGAPPPS-NH₂

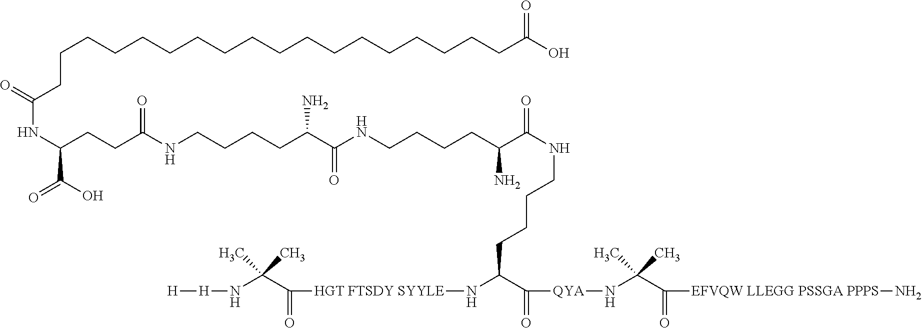

Peptide backbone: SEQ ID NO: 17, C-terminal amide
Substituent: Chem. 13 attached via ε-amino group of K16
Synthesis methods: SPPS_B; SC_B
Molecular weight (average) calculated: 5026.6 Da
LCMS_A: Rt=5.8 min; found [M+3H]³⁺ 1676.2, [M+4H]⁴⁺ 1257.5.

### Compound No. 30

### H-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-KYA-Aib-EFVQWLLEGGPSSGAPPPS-NH₂

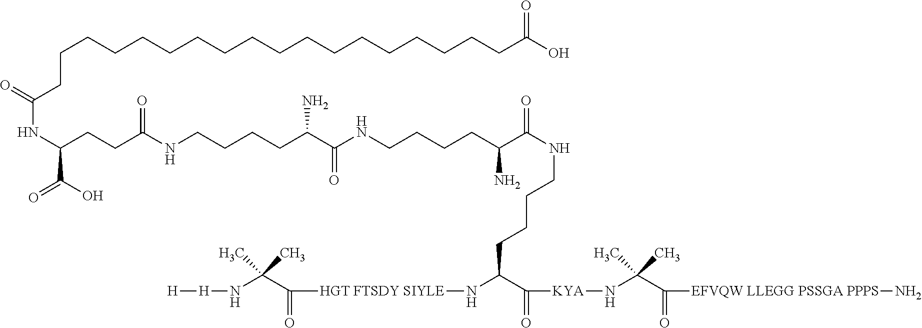

Peptide backbone: SEQ ID NO: 18, C-terminal amide
Substituent: Chem. 13 attached via ε-amino group of K16
Synthesis methods: SPPS_A; SC_A
Molecular weight (average) calculated: 4976.6 Da
LCMS_B: Rt=2.7 min; found [M+3H]³⁺ 1658.7, [M+4H]⁴⁺ 1244.3.

### Compound No. 31

### H-Aib-HGTFTSDYSYYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-KAA-Aib-EFVQWLLEGGPSSGAPPPS-NH₂

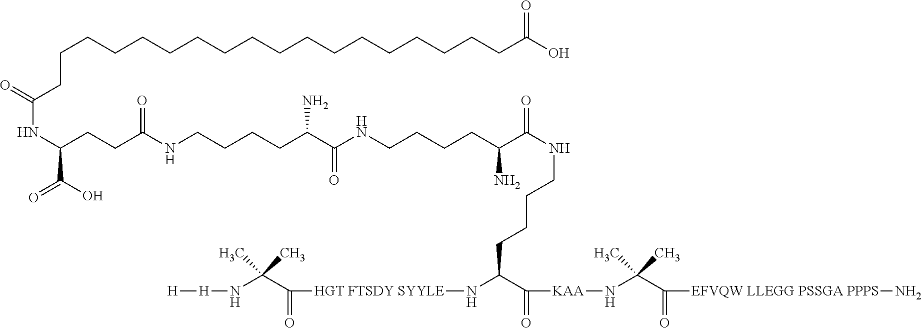

Peptide backbone: SEQ ID NO: 19, C-terminal amide
Substituent: Chem. 13 attached via ε-amino group of K16
Synthesis methods: SPPS_A; SC_A
Molecular weight (average) calculated: 4934.6 Da
LCMS_B: Rt=2.7 min; found [M+3H]³⁺ 1644.8, [M+4H]⁴⁺ 1233.8.

### Compound No. 32

### H-Aib-HGTFTSDYSYYLE-K[(2S)-2-amino-6-[[(2S)-2-amino-6-[[(4S)-4-carboxy-4-(17-carboxyheptadecanoylamino)butanoyl]amino]hexanoyl]amino]hexanoyl]-QAA-Aib-EFVQWLLEGGPSSGAPPPS-NH₂

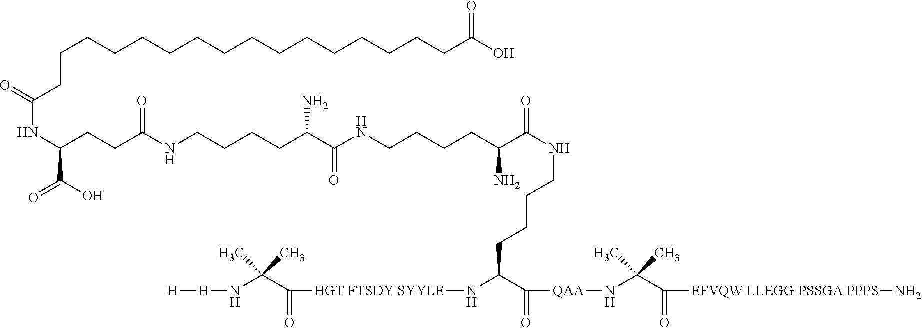

Peptide backbone: SEQ ID NO: 14, C-terminal amide
Substituent: Chem. 7 attached via ε-amino group of K16
Synthesis methods: SPPS_B; SC_B
Molecular weight (average) calculated: 4906.5 Da
LCMS_A: Rt=5.6 min; found [M+3H]³⁺ 1636.4, [M+4H]⁴⁺ 1227.3.

### Compound No. 33

### H-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-KAA-Aib-EFVQWLLEGGPSSGAPPPS-NH₂

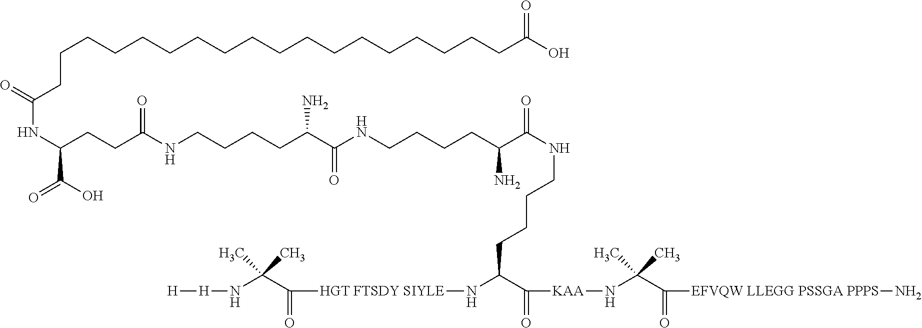

Peptide backbone: SEQ ID NO: 20, C-terminal amide
Substituent: Chem. 13 attached via ε-amino group of K16
Synthesis methods: SPPS_B; SC_B
Molecular weight (average) calculated: 4884.5 Da
LCMS_A: Rt=5.7 min; found [M+3H]³⁺ 1629.0, [M+4H]⁴⁺ 1222.0.

### Compound No. 34

### H-Aib-HGTFTSDYSIYLE-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-KAA-Aib-EFVQWLLEGGPSSGAPPPS-NH₂

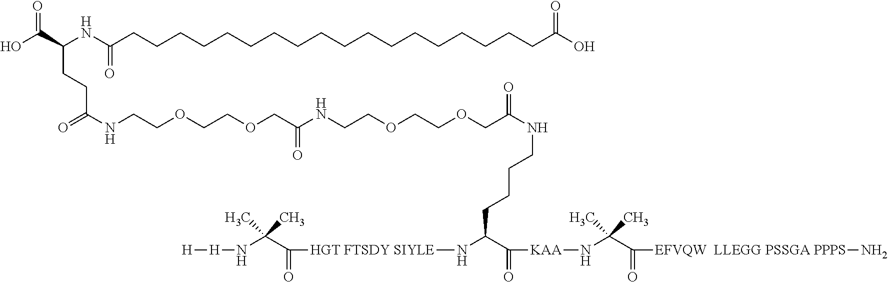

Peptide backbone: SEQ ID NO: 20, C-terminal amide
Substituent: Chem. 12 attached via ε-amino group of K16
Synthesis methods: SPPS_B; SC_B
Molecular weight (average) calculated: 4918.5 Da
LCMS_A: Rt=6.5 min; found [M+3H]³⁺ 1640.3, [M+4H]⁴⁺ 1230.4.

### Compound No. 35

### H-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QKA-Aib-EFVQWLLEGGPSSGAPPPS-NH₂

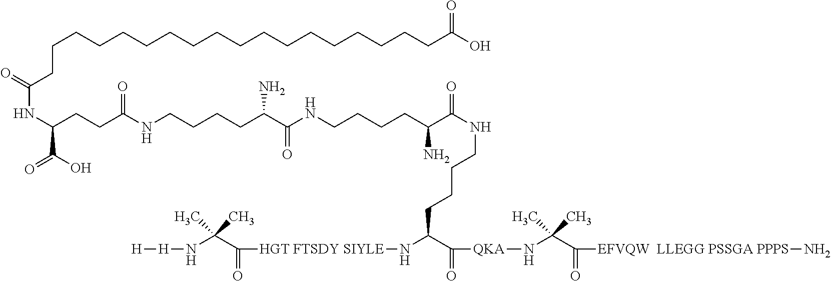

Peptide backbone: SEQ ID NO: 21, C-terminal amide
Substituent: Chem. 13 attached via ε-amino group of K16
Synthesis methods: SPPS_B; SC_B
Molecular weight (average) calculated: 4941.6 Da
LCMS_A: Rt=5.6 min; found [M+3H]³⁺ 1648.0, [M+4H]⁴⁺ 1236.2.

### Compound No. 36

### H-Aib-HGTFTSDYSKYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂

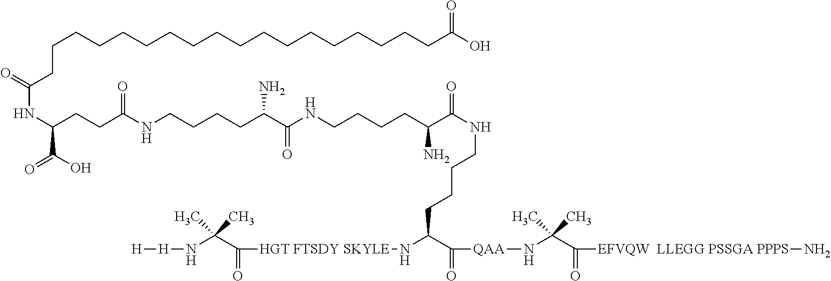

Peptide backbone: SEQ ID NO: 22, C-terminal amide
Substituent: Chem. 13 attached via ε-amino group of K16
Synthesis methods: SPPS_B; SC_B
Molecular weight (average) calculated: 4899.5 Da
LCMS_A: Rt=5.6 min; found [M+3H]³⁺ 1633.8, [M+4H]⁴⁺ 1225.7.

### Compound No. 37

### H-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-RAA-Aib-EFVQWLLEGGPSSGAPPPS-NH₂

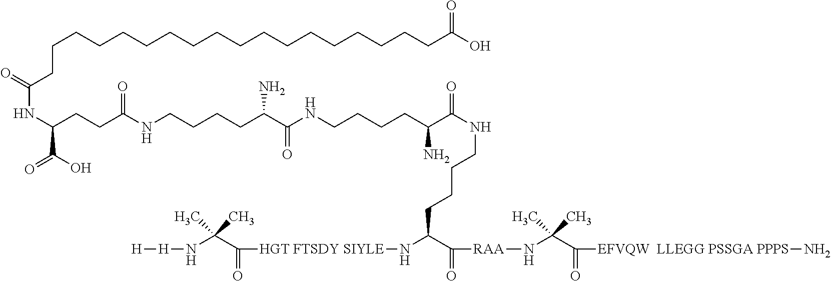

Peptide backbone: SEQ ID NO: 23, C-terminal amide
Substituent: Chem. 13 attached via ε-amino group of K16
Synthesis methods: SPPS_B; SC_A
Molecular weight (average) calculated: 4912.6 Da
LCMS_A: Rt=5.8 min; found [M+3H]³⁺ 1636.2, [M+4H]⁴⁺ 1229.0.

### Compound No. 38

### H-Aib-HGTFTSDYSIYLE-K[(2S)-2-amino-6-[[(2S)-2-amino-6-[[(4S)-4-carboxy-4-(17-carboxyheptadecanoylamino)butanoyl]amino]hexanoyl]amino]hexanoyl]-KYA-Aib-EFVQWLLEGGPSSGAPPPS-NH₂

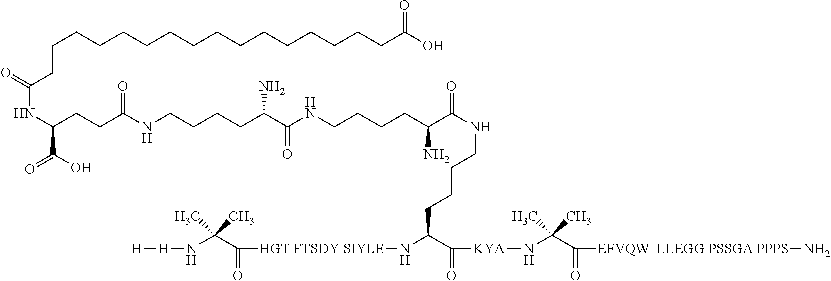

Peptide backbone: SEQ ID NO: 18, C-terminal amide
Substituent: Chem. 7 attached via ε-amino group of K16
Synthesis methods: SPPS_A; SC_A
Molecular weight (average) calculated: 4948.6 Da
LCMS_A: Rt=5.5 min; found [M+3H]³⁺ 1650.2, [M+4H]⁴⁺ 1238.0.

### Compound No. 39

### H-Aib-HGTFTSDYSIYLE-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-KYA-Aib-EFVQWLLEGGPSSGAPPPS-NH₂

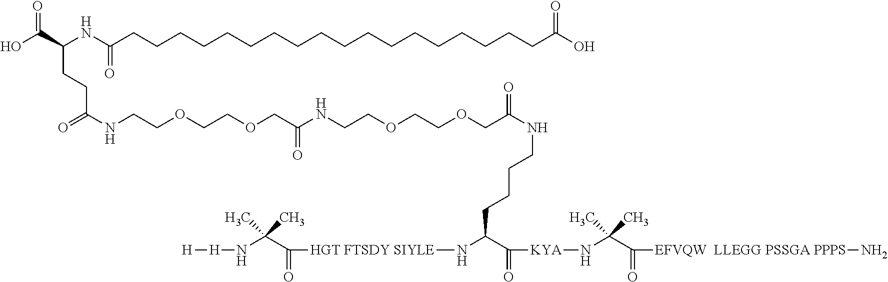

Peptide backbone: SEQ ID NO: 18, C-terminal amide
Substituent: Chem. 12 attached via ε-amino group of K16
Synthesis methods: SPPS_A; SC_A
Molecular weight (average) calculated: 5010.6 Da
LCMS_A: Rt=6.6 min; found [M+3H]³⁺ 1670.9, [M+4H]⁴⁺ 1253.7.

### Compound No. 40

### H-Aib-HGTFTSDYSYYLE-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-QAA-Aib-EFVQWLLEGGPSSGAPPPS-NH₂

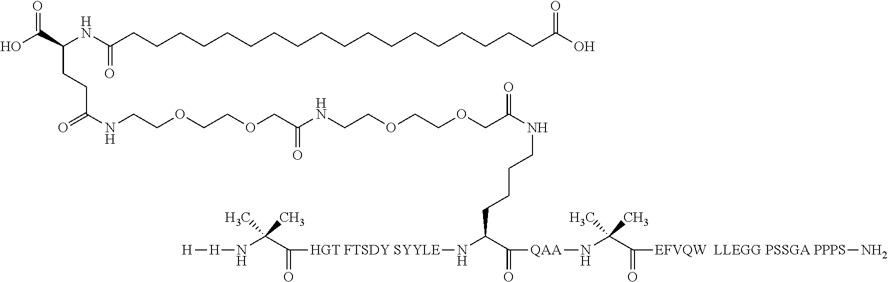

Peptide backbone: SEQ ID NO: 14, C-terminal amide
Substituent: Chem. 12 attached via ε-amino group of K16
Synthesis methods: SPPS_A; SC_A
Molecular weight (average) calculated: 4968.5 Da
LCMS_A: Rt=6.5 min; found [M+3H]³⁺ 1656.9, [M+4H]⁴⁺ 1242.9.

### Compound No. 41

### H-Aib-HGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-KYA-Aib-EFVQWLLEGGPSSGAPPPS-NH₂

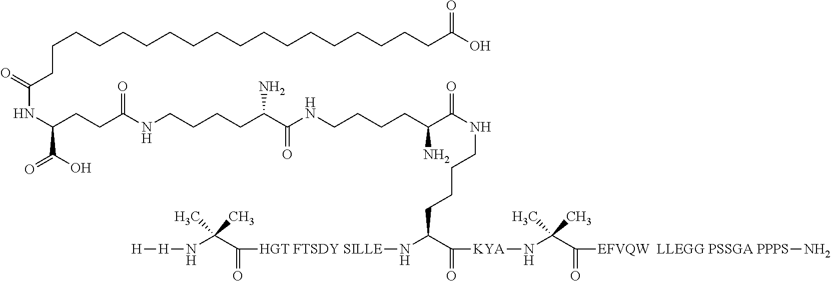

Peptide backbone: SEQ ID NO: 24, C-terminal amide
Substituent: Chem. 13 attached via ε-amino group of K16
Synthesis methods: SPPS_A; SC_A
Molecular weight (average) calculated: 4926.6 Da
LCMS_A: Rt=5.8 min; found [M+3H]³⁺ 1643.0, [M+4H]⁴⁺ 1232.5.

### Compound No. 42

### H-Aib-HGTFTSDYSYLLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂

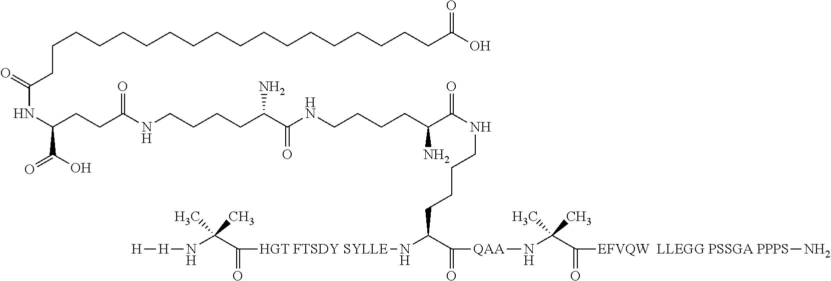

Peptide backbone: SEQ ID NO: 25, C-terminal amide
Substituent: Chem. 13 attached via ε-amino group of K16
Synthesis methods: SPPS_A; SC_A
Molecular weight (average) calculated: 4884.5 Da
LCMS_A: Rt=5.9 min; found [M+3H]³⁺ 1628.9, [M+4H]⁴⁺ 1222.0.

### Compound No. 43

### H-Aib-HGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂

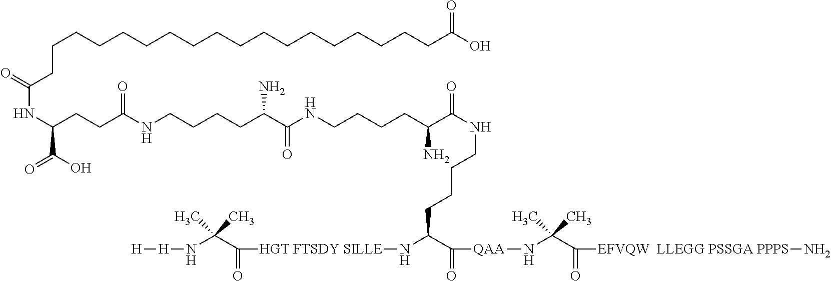

Peptide backbone: SEQ ID NO: 26, C-terminal amide
Substituent: Chem. 13 attached via ε-amino group of K16
Synthesis methods: SPPS_A; SC_A
Molecular weight (average) calculated: 4834.5 Da
LCMS_A: Rt=6.0 min; found [M+3H]³⁺ 1612.3, [M+4H]⁴⁺ 1209.5.

### Compound No. 44

### H-Aib-HGTFTSDYSIYLE-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-QAA-Aib-EFVQWLLEGGPSSGAPPPS-NH₂

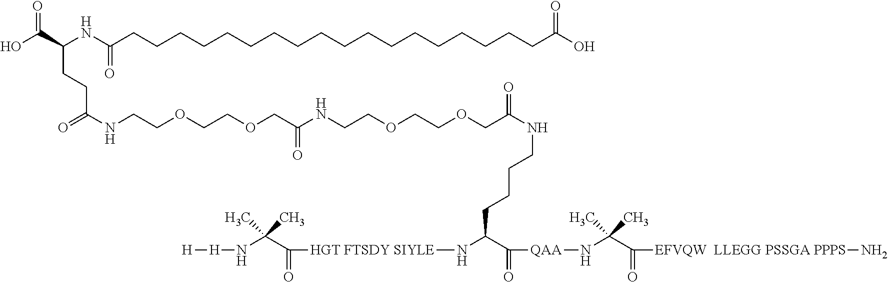

Peptide backbone: SEQ ID NO: 11, C-terminal amide
Substituent: Chem. 12 attached via ε-amino group of K16
Synthesis methods: SPPS_A; SC_A
Molecular weight (average) calculated: 4918.5 Da
LCMS_A: Rt=6.7 min; found [M+3H]³⁺ 1640.0, [M+4H]⁴⁺ 1230.3.

### Compound No. 45

### H-Aib-HGTFTSDYSILLE-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-QAA-Aib-EFVQWLLEGGPSSGAPPPS-NH₂

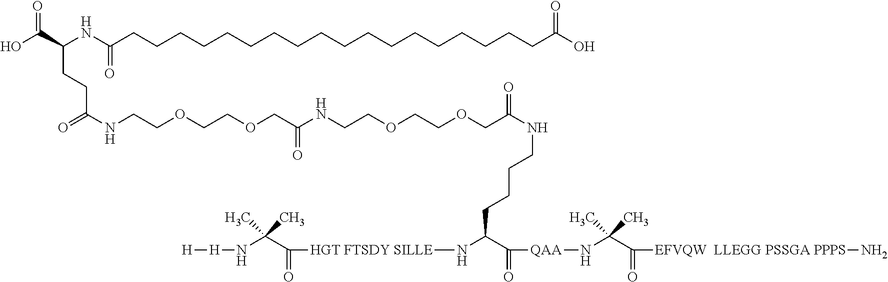

Peptide backbone: SEQ ID NO: 26, C-terminal amide
Substituent: Chem. 12 attached via ε-amino group of K16
Synthesis methods: SPPS_A; SC_A
Molecular weight (average) calculated: 4868.5 Da
LCMS_A: Rt=6.7 min; found [M+3H]³⁺ 1623.4, [M+4H]⁴⁺ 1217.9.

### Compound No. 46

### Y-Aib-QGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂

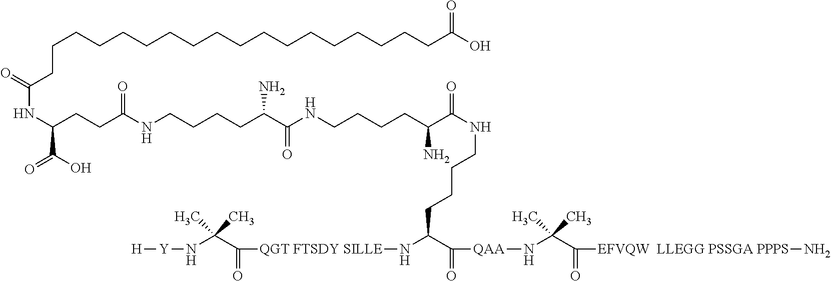

Peptide backbone: SEQ ID NO: 27, C-terminal amide
Substituent: Chem. 13 attached via ε-amino group of K16
Synthesis methods: SPPS_A; SC_A
Molecular weight (average) calculated: 4851.5 Da
LCMS_A: Rt=6.3 min; found [M+3H]³⁺ 1617.8, [M+4H]⁴⁺ 1213.6.

### Compound No. 47

### Y-Aib-QGTFTSDYSYYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂P

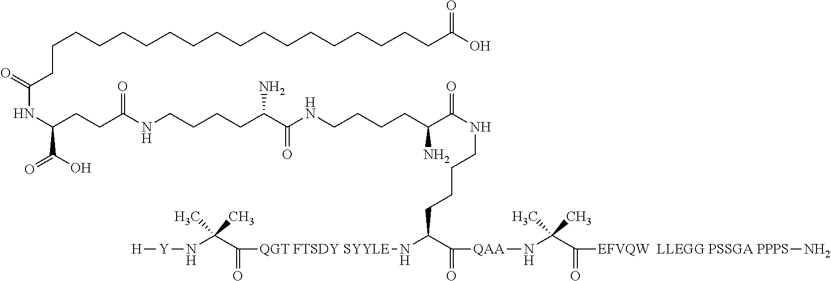

Peptide backbone: SEQ ID NO: 28, C-terminal amide
Substituent: Chem. 13 attached via ε-amino group of K16
Synthesis methods: SPPS_A; SC_A
Molecular weight (average) calculated: 4951.5 Da
LCMS_A: Rt=6.2 min; found [M+3H]³⁺ 1651.1, [M+4H]⁴⁺ 1238.7.

### Compound No. 48

### Y-Aib-QGTFTSDYSYLLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂

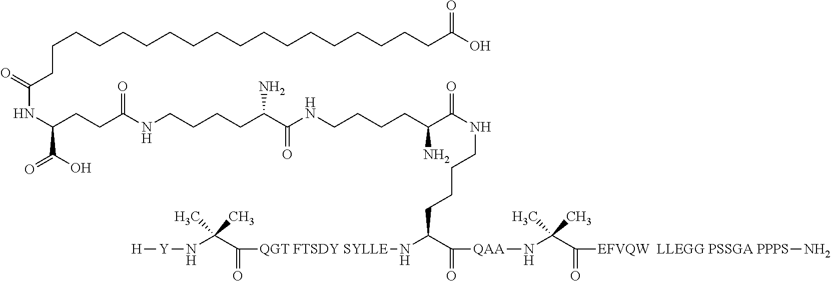

Peptide backbone: SEQ ID NO: 29, C-terminal amide
Substituent: Chem. 13 attached via ε-amino group of K16
Synthesis methods: SPPS_A; SC_A
Molecular weight (average) calculated: 4901.5 Da
LCMS_A: Rt=6.3 min; found [M+3H]³⁺ 1634.5, [M+4H]⁴⁺ 1226.3.

### Compound No. 49

### H-Aib-HGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAAEEFVQWLLEGGPSSGAPPPS-NH₂

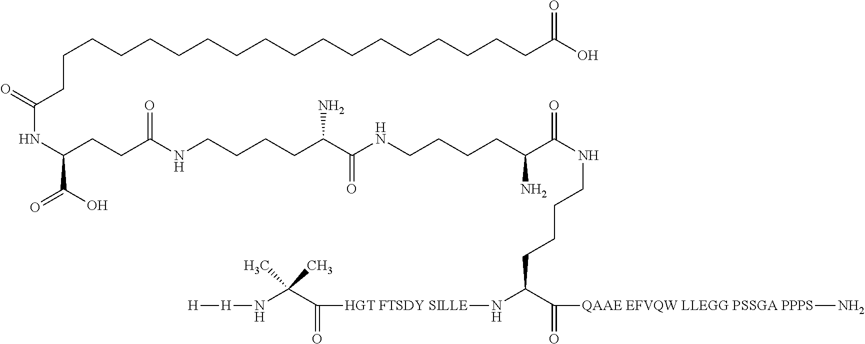

Peptide backbone: SEQ ID NO: 30, C-terminal amide
Substituent: Chem. 13 attached via ε-amino group of K16
Synthesis methods: SPPS_A; SC_A
Molecular weight (average) calculated: 4878.5 Da
LCMS_A: Rt=5.9 min; found [M+3H]³⁺ 1626.9, [M+4H]⁴⁺ 1220.3.

### Compound No. 50

### H-Aib-HGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAAEEFVQWLLEGGPSSGAPPPS-OH

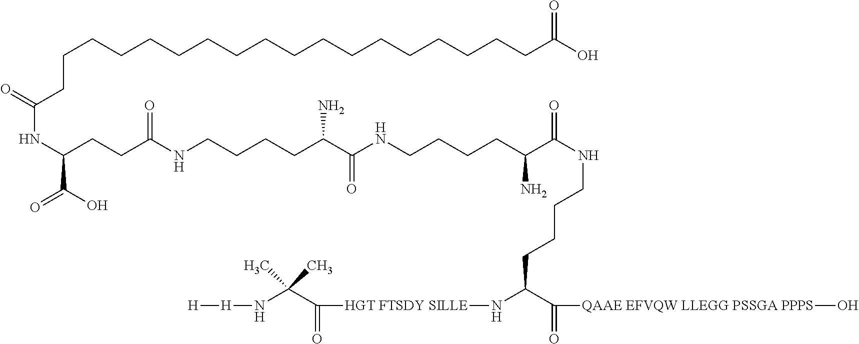

Peptide backbone: SEQ ID NO: 30, C-terminal acid
Substituent: Chem. 13 attached via ε-amino group of K16
Synthesis methods: SPPS_A; SC_A
Molecular weight (average) calculated: 4879.5 Da
LCMS_A: Rt=6.0 min; found [M+3H]³⁺ 1627.0, [M+4H]⁴⁺ 1220.8.

### Compound No. 51

### H-Aib-HGTFTSDYSILLE-K[(2S)-2-amino-6-[[(2S)-2-amino-6-[[(4S)-4-carboxy-4-(17-carboxyheptadecanoylamino)butanoyl]amino]hexanoyl]amino]hexanoyl]-QAAEEFVQWLLEGGPSSGAPPPS-OH

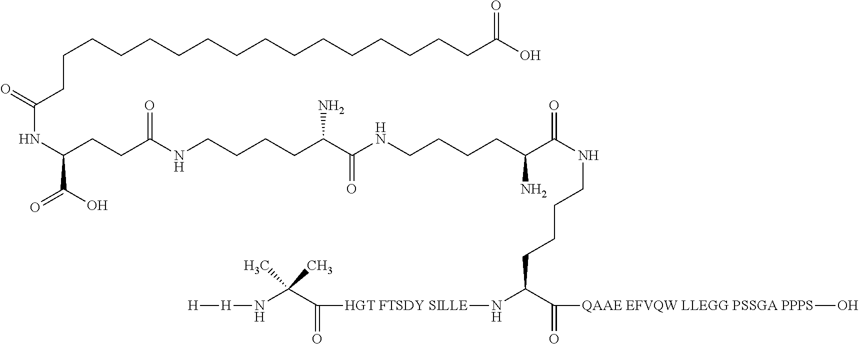

Peptide backbone: SEQ ID NO: 30, C-terminal acid
Substituent: Chem. 7 attached via ε-amino group of K16
Synthesis methods: SPPS_A; SC_A
Molecular weight (average) calculated: 4851.4 Da
LCMS_A: Rt=5.7 min; found [M+3H]³⁺ 1617.8, [M+4H]⁴⁺ 1213.7.

### Compound No. 52

### H-Aib-HGTFTSDYSILLE-K[(2S)-2-amino-6-[[(2S)-2-amino-6-[[(4S)-4-carboxy-4-(17-carboxyheptadecanoylamino)butanoyl]amino]hexanoyl]amino]hexanoyl]-QAAQEFVQWLLEGGPSSGAPPPS-OH

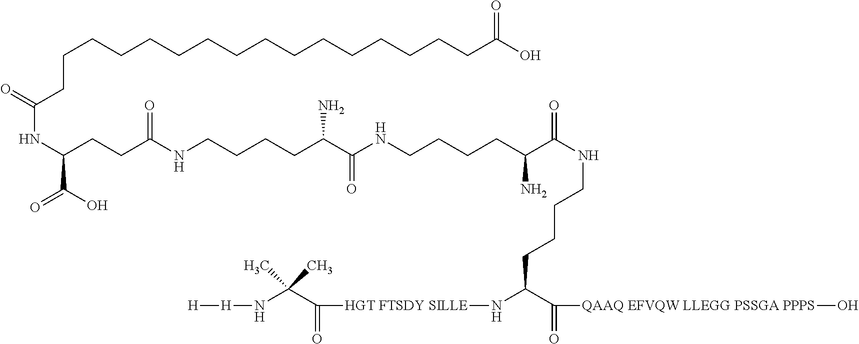

Peptide backbone: SEQ ID NO: 31, C-terminal acid
Substituent: Chem. 7 attached via ε-amino group of K16
Synthesis methods: SPPS_A; SC_A
Molecular weight (average) calculated: 4850.4 Da
LCMS_A: Rt=5.6 min; found [M+3H]³⁺ 1617.5, [M+4H]⁴⁺ 1213.4.

### Compound No. 53

### H-Aib-HGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAAQEFVQWLLEGGPSSGAPPPS-OH

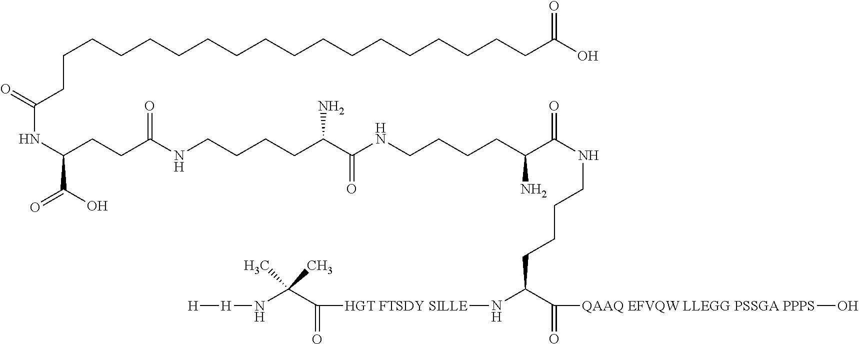

Peptide backbone: SEQ ID NO: 31, C-terminal acid
Substituent: Chem. 13 attached via ε-amino group of K16
Synthesis methods: SPPS_A; SC_A
Molecular weight (average) calculated: 4878.5 Da
LCMS_A: Rt=5.9 min; found [M+3H]³⁺ 1626.8, [M+4H]⁴⁺ 1220.3.

### Compound No. 54

### H-Aib-HGTFTSDYSILLE-K[(2S)-2-amino-6-[[(2S)-2-amino-6-[[(4S)-4-carboxy-4-(17-carboxyheptadecanoylamino)butanoyl]amino]hexanoyl]amino]hexanoyl]-QAAREFVQWLLEGGPSSGAPPPS-OH

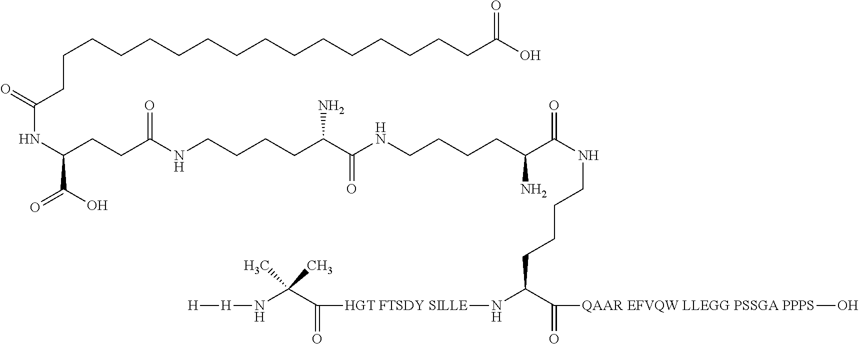

Peptide backbone: SEQ ID NO: 32, C-terminal acid
Substituent: Chem. 7 attached via ε-amino group of K16
Synthesis methods: SPPS_A; SC_A
Molecular weight (average) calculated: 4878.5 Da
LCMS_A: Rt=5.4 min; found [M+3H]³⁺ 1626.8, [M+4H]⁴⁺ 1220.4.

### Compound No. 55

### H-Aib-HGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAAREFVQWLLEGGPSSGAPPPS-OH

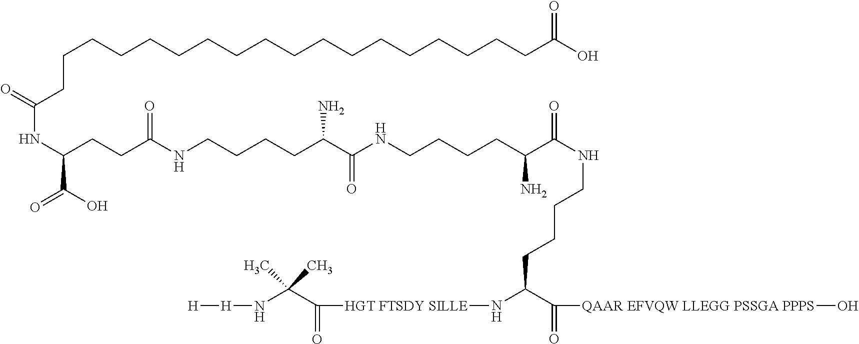

Peptide backbone: SEQ ID NO: 32, C-terminal acid
Substituent: Chem. 13 attached via ε-amino group of K16
Synthesis methods: SPPS_A; SC_A
Molecular weight (average) calculated: 4878.5 Da
LCMS_A: Rt=5.6 min; found [M+3H]³⁺ 1636.2, [M+4H]⁴⁺ 1227.4.

### Compound No. 56

### H-Aib-HGTFTSDYSILLE-K[(2S)-2-amino-6-[[(2S)-2-amino-6-[[(4S)-4-carboxy-4-(17-carboxyheptadecanoylamino)butanoyl]amino]hexanoyl]amino]hexanoyl]-QAAHEFVQWLLEGGPSSGAPPPS-OH

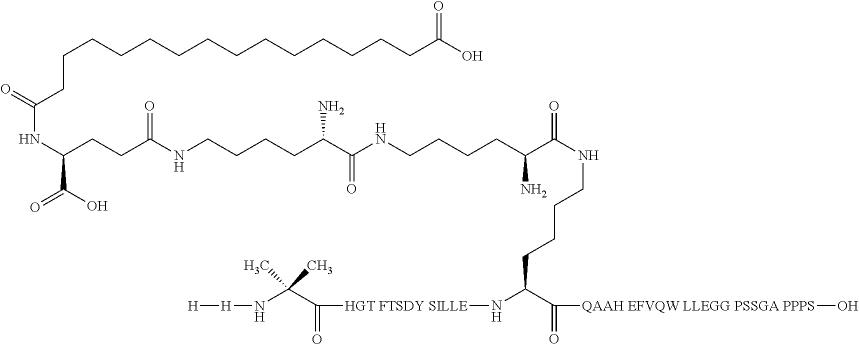

Peptide backbone: SEQ ID NO: 33, C-terminal acid
Substituent: Chem. 7 attached via ε-amino group of K16
Synthesis methods: SPPS_A; SC_A
Molecular weight (average) calculated: 4859.5 Da
LCMS_A: Rt=5.3 min; found [M+3H]³⁺ 1620.4, [M+4H]⁴⁺ 1215.7.

### Compound No. 57

### H-Aib-HGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAAHEFVQWLLEGGPSSGAPPPS-OH

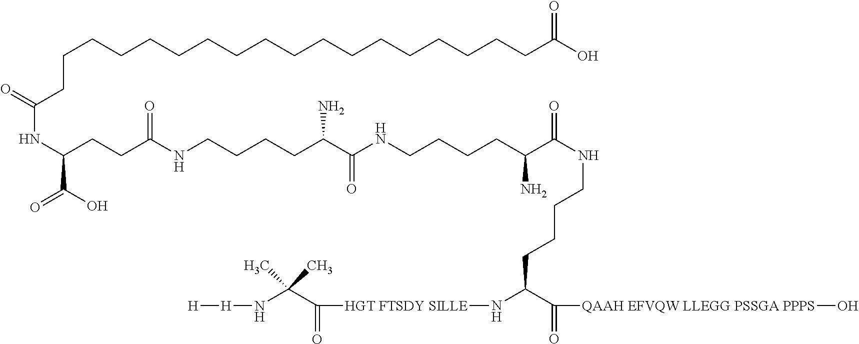

Peptide backbone: SEQ ID NO: 33, C-terminal acid
Substituent: Chem. 13 attached via ε-amino group of K16
Synthesis methods: SPPS_A; SC_A
Molecular weight (average) calculated: 4887.5 Da
LCMS_A: Rt=5.6 min; found [M+3H]³⁺ 1629.8, [M+4H]⁴⁺ 1222.7.

### Structures of Standard and Comparator Compounds:

### Native Human GLP-1 (7-37), SEQ ID NO: 35

H—H A E G T F T S D V S S Y L E G Q A A K E F I A W L V K G R G—OH

### Native Human GIP, SEQ ID NO: 36

H—Y A E G T F I S D Y S I A M D K I H Q Q D F V N W L L A Q K G K K N D W K H N I T Q-OH

### Native Human Glucagon, SEQ ID NO: 37

H—H S Q G T F T S D Y S K Y L D S R R A Q D F V Q W L M N T—OH

### Native Mouse GIP, SEQ ID NO: 34

H—Y A E G T F I S D Y S I A M D K I R Q Q D F V N W L L A Q R G K K S D W K H N I T Q-OH

### Compound No. 58

### Y-Aib-QGTFTSDYSIYLDKQAA-Aib-EFVNWLLAGGPSSGAPPPS-K[hexadecanoyl]-NH₂

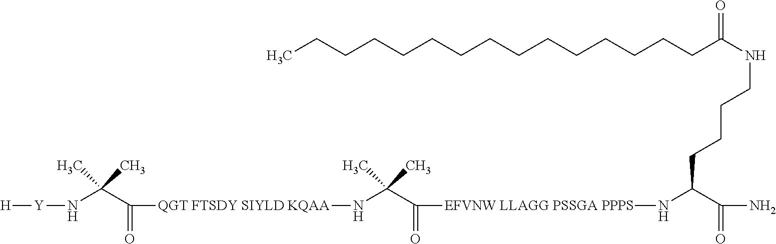

Peptide backbone: SEQ ID NO: 53, C-terminal amide
Substituent: Hexadecanoyl attached via ε-amino group of K40
Synthesis methods: see U.S. Pat. No. 9,062,124 Example 18, SEQ ID NO 124
Molecular weight (average) calculated: 4472.1 Da
LCMS_A: Rt=7.1 min; found [M+3H]³⁺ 1491.4, [M+4H]⁴⁺ 1118.9.

### Compound No. 59

### Y-Aib-QGTFTSDYSIYLDKQAA-Aib-EFVNWLLAGGPSSGAPPPS-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-NH₂

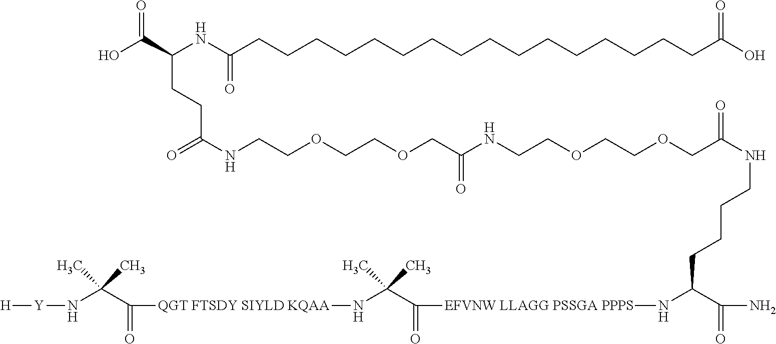

Peptide backbone: SEQ ID NO: 53, C-terminal amide
Substituent: Chem. 6 attached via ε-amino group of K40
Synthesis methods: SPPS_A; SC_A
Molecular weight (average) calculated: 4949.5 Da
LCMS_A: Rt=6.1 min; found [M+3H]³⁺ 1650.4, [M+4H]⁴⁺ 1238.2.

### Compound No. 60

### Y-Aib-QGTFTSDYSIYLDKQAA-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-EFVNWLLAGGPSSGAPPPS-NH₂

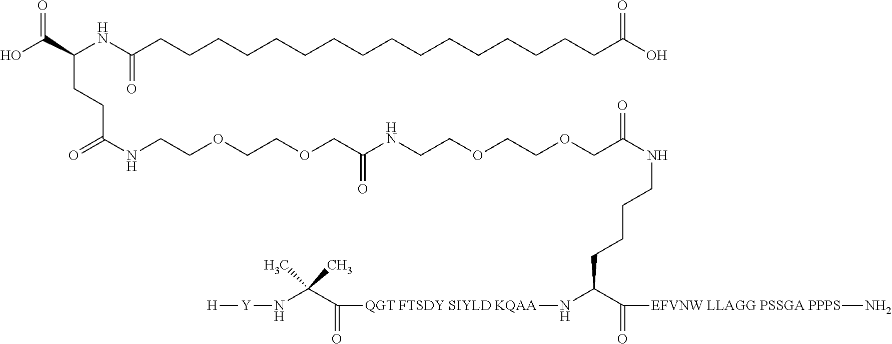

Peptide backbone: SEQ ID NO: 38, C-terminal amide
Substituent: Chem. 6 attached via ε-amino group of K20
Synthesis methods: SPPS_A; SC_B
Molecular weight (average) calculated: 4864.4 Da
LCMS_A: Rt=6.1 min; found [M+3H]³⁺ 1622.1, [M+4H]⁴⁺ 1216.8.

### Compound No. 61

### Y-Aib-QGTFTSDYSYLA-K[2-[2-[2-[[2-[2-[2-[[(4)-4-carboxy-4-(17-carboxyheptadecanoylamino)butanoyl]amino]ethoxy]ethoxy]acetyl]amino]ethoxy]ethoxy]acetyl]-YLDKQAA-Aib-EFVNWLLAGGPSSGAPPPS-NH₂

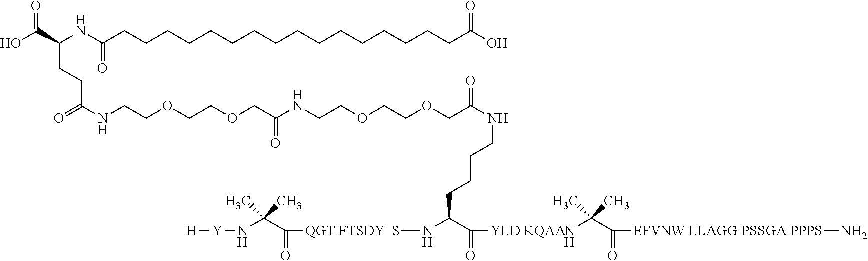

Peptide backbone: SEQ ID NO: 39, C-terminal amide
Substituent: Chem. 6 attached via ε-amino group of K12
Synthesis methods: SPPS_A; SC_A
Molecular weight (average) calculated: 4836.4 Da
LCMS_A: Rt=5.9 min; found [M+3H]³⁺ 1612.7, [M+4H]⁴⁺ 1210.0.

### Compound No. 62

### Y-Aib-QGTFTSDYSI-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-LDKQAA-Aib-EFVNWLLAGGPSSGAPPPS-NH₂

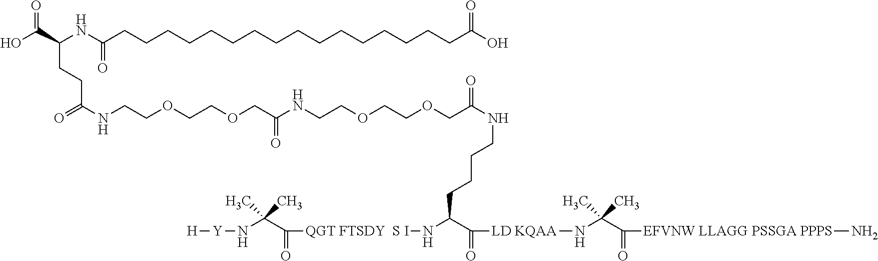

Peptide backbone: SEQ ID NO: 40, C-terminal amide
Substituent: Chem. 6 attached via ε-amino group of K13
Synthesis methods: SPPS_A; SC_A
Molecular weight (average) calculated: 4786.3 Da
LCMS_A: Rt=6.0 min; found [M+3H]³⁺ 1596.2, [M+4H]⁴⁺ 1197.4.

### Compound No. 63

### Y-Aib-QGTFTSDYSIY-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-DKQAA-Aib-EFVNWLLAGGPSSGAPPPS-NH₂

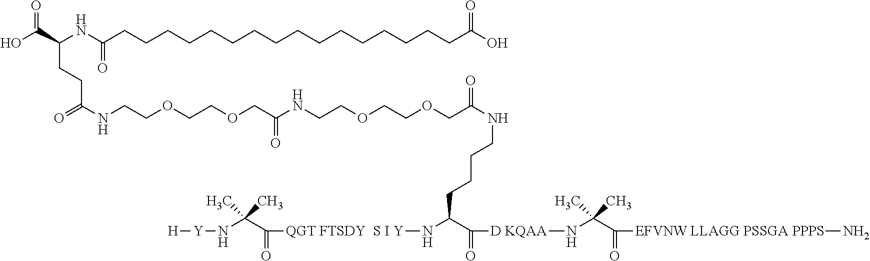

Peptide backbone: SEQ ID NO: 41, C-terminal amide
Substituent: Chem. 6 attached via ε-amino group of K14
Synthesis methods: SPPS_A; SC_A
Molecular weight (average) calculated: 4836.4 Da
LCMS_A: Rt=5.9 min; found [M+3H]³⁺ 1613.0, [M+4H]⁴⁺ 1209.9.

### Compound No. 64

### Y-Aib-QGTFTSDYSIYLDKQAA-Aib-EFV-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-WLLAGGPSSGAPPPS-NH₂

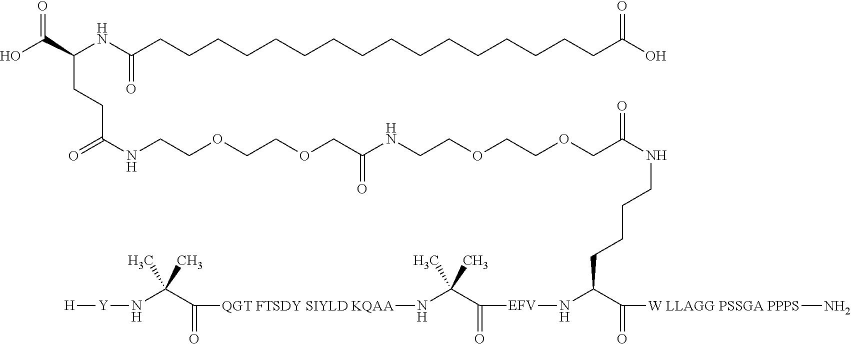

Peptide backbone: SEQ ID NO: 42, C-terminal amide
Substituent: Chem. 6 attached via ε-amino group of K24
Synthesis methods: SPPS_A; SC_A
Molecular weight (average) calculated: 4835.4 Da
LCMS_A: Rt=6.2 min; found [M+3H]³⁺ 1612.6, [M+4H]⁴⁺ 1209.6.

### Compound No. 65

### Y-Aib-QGTFTSDYSIYLDKQAA-Aib-EFVNWLLA-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-GPSSGAPPPS-NH₂

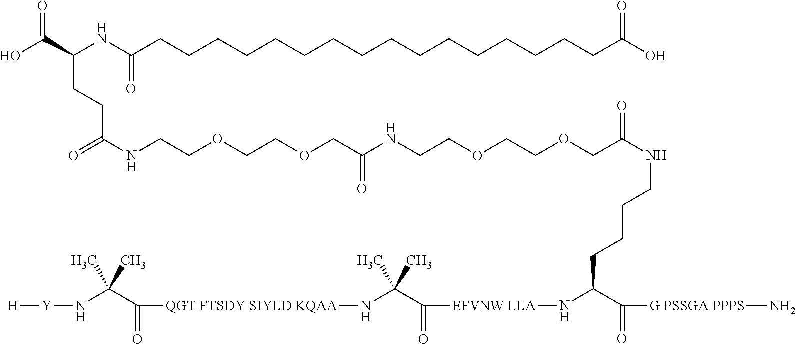

Peptide backbone: SEQ ID NO: 43, C-terminal amide
Substituent: Chem. 6 attached via ε-amino group of K29
Synthesis methods: SPPS_A; SC_A
Molecular weight (average) calculated: 4892.5 Da
LCMS_A: Rt=5.8 min; found [M+3H]³⁺ 1631.5, [M+4H]⁴⁺ 1223.9.

### Compound No. 66

### Y-Aib-QGTFTSDYSIYLDKQAA-Aib-EFVNWLLAGGPSSG-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-PPPS-NH₂

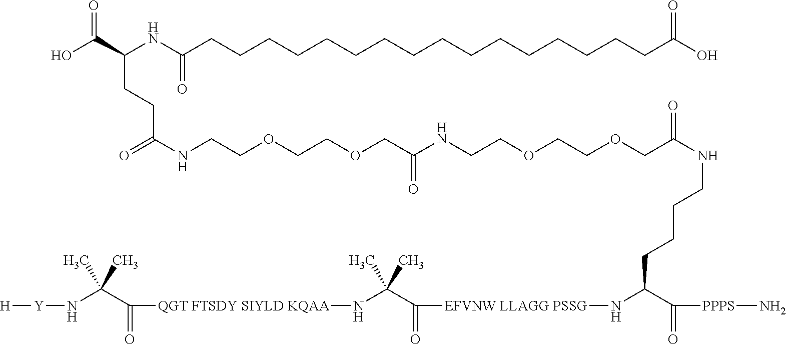

Peptide backbone: SEQ ID NO: 44, C-terminal amide
Substituent: Chem. 6 attached via ε-amino group of K35
Synthesis methods: SPPS_A; SC_A
Molecular weight (average) calculated: 4878.4 Da
LCMS_A: Rt=6.2 min; found [M+3H]³⁺ 1626.8, [M+4H]⁴⁺ 1220.5.

### Example 2: In Vitro Functional Potency (CRE Luciferase; Whole Cells)

The purpose of this example is to test the functional activity, or potency, of the compounds in vitro at the human and mouse GLP-1, GIP, and Gcg receptors. The in vitro functional potency is the measure of target receptor activation in a whole cell assay. The potencies of the compounds listed in Example 1 were determined as described below. Human GLP-1(7-37) (SEQ ID NO: 35) (identical to mouse GLP-1(7-37)), human GIP (SEQ ID NO: 36), and human glucagon (SEQ ID NO: 37) (identical to mouse glucagon) were included in appropriate assays for comparison.

### Principle

In vitro functional potency was determined by measuring the response of the target receptor in a reporter gene assay in individual cell lines. The assay was performed in stably transfected BHK cell lines that expresses one of the following G-protein coupled receptors: human GLP-1 receptor, human GIP receptor, human Gcg receptor, mouse GLP-1 receptor, mouse GIP receptor, or mouse Gcg receptor; and where each cell line contains the DNA for the cAMP response element (CRE) coupled to a promoter and the gene for firefly luciferase (CRE luciferase). When the respective receptor is activated, it results in the production of cAMP, which in turn results in expression of the luciferase protein. When assay incubation is completed, luciferase substrate (luciferin) is added resulting in the enzymatic conversion of luciferin to oxyluciferin and producing bioluminescence. The luminescence is measured as the readout for the assay.

### Cell Culture and Preparation

The cells lines used in these assays were BHK cells with BHKTS13 as a parent cell line. The cell lines were derived from a clone containing the CRE luciferase element and were established by further transfection with the respective receptor to obtain the relevant cell line. The following cell lines were used:

| Assay | Cell line |
|---|---|
| GLP-1 receptor assay | BHK CRE luc2P hGLP-1R |
| GIP receptor assay | BHK CRE luc2P hGIPR |
| Glucagon receptor assay | BHK CRE luc2P hGcgR |

The cells were cultured at 37° C. with 5% CO₂ in Cell Culture Medium. They were aliquoted and stored in liquid nitrogen. The cells were kept in continuous culture and were seeded out the day before each assay.

### Materials

The following chemicals were used in the assay: Pluronic F-68 10% (Gibco 2404), 10% fetal bovine serum (FBS; Invitrogen 16140-071), chicken egg white ovalbumin (Sigma A5503), DMEM w/o phenol red (Gibco 21063-029), DMEM (Gibco 12430-054), 1 M Hepes (Gibco 15630), Glutamax 100× (Gibco 35050), G418 (Invitrogen 10131-027), hygromycin (Invitrogen 10687-010), and steadylite plus (PerkinElmer 6016757).

### Buffers

GLP-1R and GcgR Cell Culture Medium consisted of DMEM medium with 10% FBS, 500 pg/mL G418, and 300 pg/mL hygromycin. GIPR Cell Culture Medium consisted of DMEM medium with 10% FBS, 400 pg/mL G418, and 300 pg/mL hygromycin. Assay Buffer consisted of DMEM w/o phenol red, 10 mM Hepes, 1× Glutamax, 1% ovalbumin, and 0.1% Pluronic F-68. The Assay Buffer was mixed 1:1 with an equal volume of the test compound in Assay Buffer to give the final assay concentration.

### Procedure

1) Cells were plated at 5000 cells/well and incubated overnight in the assay plate.
2) Cells were washed once in DPBS.
3) Stocks of the test compounds and reference compounds in concentrations ranging from 100-300 pM were diluted 1:150 in Assay Buffer. Compounds were then diluted 1:10 in column 1 of a 96 deep well dilution plate and then carried across the row creating a 3.5 fold, 12 point dilution curve.
4) Assay Buffer (50 μl aliquot) was added to each well in the assay plate.
5) A 50 μl aliquot of compound or blank was transferred from the dilution plate to the assay plate containing the Assay Buffer.
6) The assay plate was incubated for 3 h in a 5% CO₂ incubator at 37° C.
7) The cells were washed once with DPBS.
8) A 100 μl aliquot of DPBS was added to each well of the assay plate.
9) A 100 μl aliquot of steadylite plus reagent (light sensitive) was added to each well of the assay plate.
10) Each assay plate was covered with aluminum foil to protect it from light and shaken at 250 RPM for 30 min at room temperature.
11) Each assay plate was read in a microtiter plate reader.

### Calculations and Results

The data from the microtiter plate reader was first regressed in an Excel in order to calculate the x-axis, log scale concentrations based on the individual test compound's stock concentration and the dilutions of the assay. This data was then transferred to GraphPad Prism software for graphing and statistical analysis. The software performs a non-linear regression (log(agonist) vs response). EC₅₀ values which were calculated by the software and reported in pM are shown in Tables 1 and 2 below. A minimum of two replicates was measured for each sample. The reported values are averages of the replicates.

TABLE 1
Functional potencies at human GLP-1R, GIPR, and GcgR for triple
agonists varying the position of the substituent.

| Com- | | | hGLP-1R | hGIPR | hGcgR |
|---|---|---|---|---|---|
| pound | | Position of | CRE Luc | CRE Luc | CRE Luc |
| No. | Substituent | substituent | EC₅₀ (pM) | EC₅₀ (pM) | EC₅₀ (pM) |
| hGLP- | — | — | 6.9 | nd | nd |
| 1(7-37) | | | | | |
| hGIP | — | — | nd | 10.4 | nd |
| hGcg | — | — | nd | nd | 9.5 |
| 58 | hexadecanoyl | Lys40 | 4.1 | 7.2 | 21.2 |
| 59 | Chem. 6 | Lys40 | 11.7 | 44.5 | 703.4 |
| 61 | Chem. 6 | Lys12 | 6.0 | 43.0 | 321.0 |
| 62 | Chem. 6 | Lys13 | 36.0 | 65.0 | 891.0 |
| 63 | Chem. 6 | Lys14 | 25.0 | 175.0 | 1112.0 |
| 1 | Chem. 6 | Lys16 | 8.6 | 23.7 | 157.4 |
| 2 | Chem. 6 | Lys17 | 7.1 | 49.8 | 47.6 |
| 60 | Chem. 6 | Lys20 | 14.9 | 416.1 | 182.0 |
| 3 | Chem. 6 | Lys21 | 5.4 | 27.5 | 34.7 |
| 64 | Chem. 6 | Lys24 | 45.4 | 173.9 | 464.5 |
| 65 | Chem. 6 | Lys29 | 132.0 | 56.0 | 1347.0 |
| 66 | Chem. 6 | Lys35 | 12.0 | 26.0 | 1023.0 |

nd = not determined.

Compound No. 58 is a known (U.S. Pat. No. 9,062,124 Example 18, SEQ ID NO 124) and potent triple agonist with a hexadecanoyl moiety attached to the epsilon-amino group of Lys40. It was expected that exchanging the substituent at Lys40 to substituent Chem. 6 (i.e. Compound No. 59) would result in an equipotent compound with a longer half-life. However, the above results show that Compound No. 59 has a lower potency at all three receptors, especially at the GIPR and GcgR. Accordingly, the substituent cannot be changed from a fatty monoacid to a fatty diacid and similar potency obtained. The results in Table 1 indicate that placement of the fatty diacid-based substituent to a lysine E-amine is preferred when the lysine is located at positions 16, 17, or 21 in the peptide backbone, whereas significant reductions in potency at one or more receptors is seen when the 018 diacid-based substituent Chem. 6 is in any of positions 12, 13, 14, 20, 24, 29, 35, or 40.

TABLE 2
Functional potencies at human GLP-1R, GIPR, and
GcgR for triple agonists of the invention wherein the
substituent is present in positions 16, 17, or 21.

| | hGLP-1R | hGIPR | hGcgR |
|---|---|---|---|
| Compound | CRE Luc | CRE Luc | CRE Luc |
| No. | EC₅₀ (pM) | EC₅₀ (pM) | EC₅₀ (pM) |
| hGLP-1(7-37) | 6.9 | nd | nd |
| hGIP | nd | 10.4 | nd |
| hGcg | nd | nd | 9.5 |
| 1 | 8.6 | 23.7 | 157.4 |
| 2 | 7.1 | 49.8 | 47.6 |
| 3 | 5.4 | 27.5 | 34.7 |
| 4 | 9.4 | 60.3 | 31.4 |
| 5 | 7.2 | 20.6 | 205.0 |
| 6 | 5.1 | 7.7 | 216.2 |
| 7 | 8.2 | 65.5 | 61.3 |
| 8 | 12.1 | 45.5 | 91.9 |
| 9 | 4.4 | 48.6 | 116.0 |
| 10 | 4.7 | 27.6 | 249.8 |
| 11 | 4.3 | 7.9 | 129.1 |
| 12 | 2.4 | 1.8 | 239.3 |
| 13 | 2.8 | 2.1 | 157.2 |
| 14 | 4.3 | 4.2 | 286.8 |
| 15 | 1.4 | 4.8 | 267.2 |
| 16 | 3.1 | 11.4 | 185.0 |
| 17 | 2.8 | 7.0 | 86.6 |
| 18 | 106.4 | 7.1 | 72.1 |
| 19 | 1.7 | 55.0 | 357.1 |
| 20 | 5.1 | 8.1 | 147.2 |
| 21 | 3.0 | 3.2 | 26.6 |
| 22 | 2.0 | 7.1 | 48.7 |
| 23 | 2.0 | 5.1 | 18.3 |
| 24 | 6.8 | 7.7 | 13.1 |
| 25 | 4.6 | 6.9 | 25.2 |
| 26 | 3.1 | 6.5 | 13.6 |
| 27 | 0.8 | 2.5 | 55.0 |
| 28 | 4.5 | 7.7 | 16.7 |
| 29 | 5.8 | 7.2 | 9.6 |
| 30 | 3.9 | 15.6 | 9.3 |
| 31 | 3.0 | 9.0 | 7.7 |
| 32 | 1.6 | 3.5 | 10.3 |
| 33 | 3.3 | 7.3 | 13.7 |
| 34 | 2.6 | 8.7 | 24.7 |
| 35 | 5.2 | 13.9 | 5.0 |
| 36 | 1.8 | 9.3 | 11.4 |
| 37 | 1.8 | 10.8 | 11.8 |
| 38 | 2.8 | 13.1 | 6.4 |
| 39 | 4.4 | 17.9 | 12.9 |
| 40 | 3.7 | 5.7 | 36.4 |
| 41 | 4.3 | 9.3 | 7.6 |
| 42 | 1.9 | 3.8 | 8.0 |
| 43 | 3.0 | 2.8 | 10.8 |
| 44 | 1.8 | 6.1 | 45.2 |
| 45 | 2.4 | 1.9 | 6.8 |
| 46 | 8.3 | 3.0 | 25.0 |
| 47 | 5.6 | 14.9 | 28.7 |
| 48 | 10.1 | 9.2 | 12.6 |
| 49 | 1.4 | 2.1 | 3.5 |
| 50 | 1.0 | 1.9 | 18.5 |
| 51 | 0.7 | 1.8 | 79.9 |
| 52 | 0.8 | 6.6 | 14.9 |
| 53 | 2.7 | 4.8 | 22.0 |
| 54 | 1.5 | 28.0 | 22.8 |
| 55 | 2.0 | 7.5 | 18.8 |
| 56 | 1.1 | 2.3 | 6.4 |

nd = not determined.

The results in Table 2 show that the compounds of the present invention display potent functional activation of the human GLP-1R, human GIPR, and human GcgR, which is obtained by the peptide backbone in combination with the substituent in positions 16, 17 or 21.

### Example 3: Pharmacokinetic Study in Minipigs

The purpose of this example is to determine the half-life in vivo of the triple agonists as described herein after i.v. administration to minipigs, i.e. the prolongation of their time in the body and thereby their time of action. This is done in a pharmacokinetic (PK) study, where the terminal half-life of the triple agonist in question is determined. By terminal half-life is generally meant the period of time it takes to halve a certain plasma concentration, measured after the initial distribution phase.

### Study

Female Göttingen minipigs were obtained from Ellegaard Göttingen Minipigs (Dalmose, Denmark) approximately 7-14 months of age and weighing from approximately 16-35 kg were used in the studies. The minipigs were housed individually and fed restrictedly once daily with SDS minipig diet (Special Diets Services, Essex, UK).

After at 3 weeks of acclimatisation two permanent central venous catheters were implanted in vena cava caudalis in each animal. The animals were allowed 1 week recovery after the surgery, and were then used for repeated pharmacokinetic studies with a suitable wash-out period between successive dosings.

The animals were fasted for approximately 18 hours before dosing and from 0 to 4 hours after dosing, but had adlibitum access to water during the whole period.

The sodium salts of triple agonists of Examples 1 were dissolved to a concentration of 20-40 nmol/mL in a buffer containing 0.025% (v/v) polysorbate 20, 10 mM sodium phosphate, 250 mM glycerol, pH 7.4. Intravenous injections (the volume corresponding to usually 1.5-2 nmol/kg, for example 0.1 mL/kg) of the triple agonists were given through one catheter, and blood was sampled at predefined time points for up to 14 days post dosing (preferably through the other catheter). Blood samples (for example 0.8 mL) were collected in 8 mM EDTA buffer and then centrifuged at 4° C. and 1942 g for 10 minutes.

### Sampling and Analysis

Plasma was pipetted into Micronic tubes on dry ice and kept at −20° C. until analysed for plasma concentration of the triple agonists using ELISA, or a similar antibody-based assay, or LCMS. Individual plasma concentration-time profiles were analysed by a non-compartmental model in Phoenix WinNonLin ver. 6.4. (Pharsight Inc., Mountain View, CA, USA), and the resulting terminal half-lives (harmonic mean) determined.

### Results

TABLE 3
Terminal half-life as measured after i.v.
administration to minipigs

| Compound No. | t_{1/2} (h) |
|---|---|
| 1 | 61 |
| 2 | 45 |
| 3 | 33 |
| 5 | 85 |
| 6 | 83 |
| 7 | 79 |
| 8 | 81 |
| 9 | 103 |
| 24 | 82 |
| 25 | 79 |
| 26 | 80 |
| 30 | 78 |
| 31 | 55 |
| 32 | 52 |
| 33 | 47 |
| 39 | 66 |
| 41 | 69 |
| 42 | 66 |
| 43 | 71 |

The tested triple agonists as described herein have very long half-lives as compared to native hormones. The half-lives of hGLP-1 and hGIP as measured in man are reported to be approximately 2-4 min and 5-7 min, respectively (Meier et al., Diabetes, 2004, 53(3): 654-662). The half-life of glucagon as measured in man is reported to vary between 5 min and 30 min, depending on the route of administration (Pontiroli et al., Eur J Clin Pharmacol 1993, 45: 555-558).

### Example 4: Pharmacodynamic Study Studies in Diet-Induced Obese (DIO) Mice

The purpose of this example is to assess the in vivo effect of selected triple agonists on pharmacodynamic parameters in diet-induced obese (DIO) mice. The animals were treated once daily via subcutaneous injection with a liquid formulation of the triple agonist to be tested to assess effects on body weight, foot intake, and glucose tolerance.

### Animals and Diet

C57BL/6J male mice were purchased from Jackson Laboratories at approximately 8 weeks of age. Mice were group housed and fed a high-fat, high-sugar diet from Research Diets (D12331). Mice were maintained on this diet for 12 weeks prior to initializing the pharmacology studies. Mice exceeding a measured body weight of 50 grams were considered diet-induced obese (DIO) and included in pharmacology studies. Mice were exposed to a controlled 12 h:12 h light:dark cycle at ambient room temperature (22° C.) with ab libitum access to food and water. Studies were approved by and performed according to the guidelines of the Institutional Animal Care and Use Committee of the University of Cincinnati.

### Dosing and Formulation

All compounds in the study were formulated in the following buffer: 50 mM phosphate; 70 mM sodium chloride; 0.05% Tween-80, pH 7.4. Dosing solutions were formulated in glass vials and stored at 2-8° C. Dosing solutions were brought to room temperature before dosing and returned to 2-8° C. after dosing.

DIO mice were distributed into groups (n=8 per group) such that statistical variations in the mean and standard deviations of fat mass and body weight were minimized between groups. The animals were grouped to receive treatment as follows: Vehicle or GLP-1/GIP/Gcg receptor triple agonists as described herein, where vehicle is 50 mM phosphate, 70 mM sodium chloride; 0.05% (v/v) Tween-80, pH 7.4. The test compounds were dissolved in the vehicle, to stock concentrations of 100 pM, then diluted 50- to 200-fold in the vehicle to achieve the desired dosing solution concentrations. Animals were dosed subcutaneously once daily in the morning for each day of treatment with dosing solution at a volume of 2-5 μL per gram of body weight as necessary to achieve the desired dose (e.g. 0.3 nmol/kg, 1.0 nmol/kg, or 3.0 nmol/kg).

### Body Weight and Food Intake

Body weight (BW) and food intake were measured immediately prior to dosing each day. The percent change in body was calculated individually for each mouse based on initial body weight prior to the first injection.

### IPGTT (Intraperitoneal Glucose Tolerance Test)

On the day of the glucose tolerance test, animals were fasted for 4 h. Food was removed and animals were transferred to fresh cages. Animals had access to water but not to food. Tail blood glucose levels were measured, and mice were injected (t=0) with an intraperitoneal (i.p.) glucose load of 2 g/kg (200 mg/ml glucose solution, dose volume 10 ml/kg). Tail blood glucose levels were measured at times 0, 15, 30, 60, 90, 120 minutes following the i.p. glucose load. Stratification of the animals during the IPGTT was such that for example two mice from group 1 are dosed followed by two mice from group 2, 3, 4, before the next two mice from group 1, 2, 3 etc. were handled. This was to allow for equal distribution of “time of day” throughout all groups.

### Results:

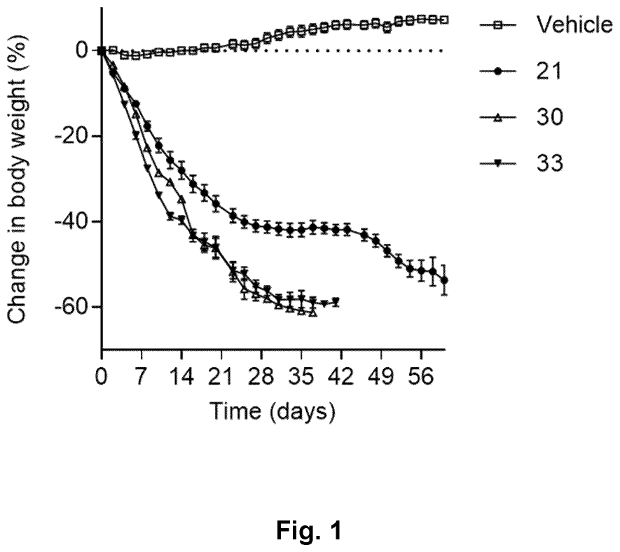

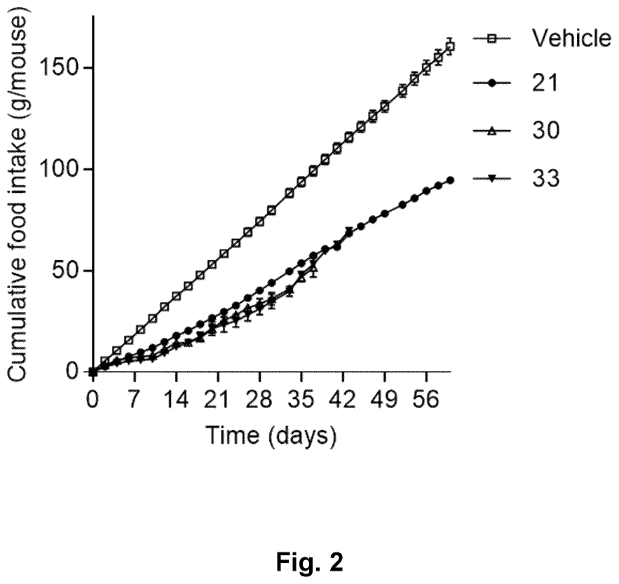

In one study, DIO mice received a daily subcutaneous dose for up to 60 days of one of the following: vehicle, Compound No. 21, Compound No. 30, or Compound No. 33. The dose of each compound was up-titrated to maximal efficacy based on the titration schedule included in Table 4. Mice that achieved body weight normalization, defined as a body weight of 22.5 g or less, were voluntarily removed from the study. Results are shown in Table 5 and FIG. 1-2. All triple agonist compounds tested resulted is substantially greater reductions of body weight and food intake than vehicle. At time points prior to day 42 (see FIG. 1), treatment with Compound No. 21 resulted in similar reduction of food intake but inferior reduction in body weight compared to Compound No. 30 and Compound No. 33. This may be explained by the similar GLP-1R and GIPR in vitro potency but reduced in vitro GcgR potency of Compound No. 21 compared to Compound No. 30 and Compound No. 33 when investigated with the protocol described in Example 2 except using the mouse-specific receptors. In vitro potencies at mouse GLP-1R, mouse GIPR and mouse GcgR are shown in Table 6.

TABLE 4
Dose titration schedule for DIO mouse study described in Table 5.

| | Dose during specified treatment day range (nmol/kg) | | | | | | | |
|---|---|---|---|---|---|---|---|---|
| Compound | Day | Day | Day | Day | Day | Day | Day | Day |
| No. | 0-1 | 2-3 | 4-7 | 8-16 | 17-23 | 24-42 | 43-47 | 48-60 |
| 21 | 1.0 | 2.0 | 3.0 | 5.0 | 10.0 | 10.0 | 20.0 | 30.0 |
| 30, 33 | 1.0 | 2.0 | 3.0 | 3.0 | 5.0 | 10.0 | — | — |

Table 5: Effects on food intake and body weight in 010 mice treated daily for up to 60 days with vehicle or a GLP-1/GIP/Gcg triple agonist following the dose titration schedule shown in

TABLE 4
The last day any mouse received treatment in that group before
being removed from the study is also included.

| | | Average | Absolute | | Change in |
|---|---|---|---|---|---|
| Com- | Last | food intake | BW (grams) | | BW (%) |
| pound | day of | per mouse | | Last day of | Last day of |
| No. | treatment | (grams/day) | Day 0 | treatment | treatment |
| Vehicle | 60 | 2.68 ± 0.07 | 56.7 ± 2.5 | 60.8 ± 2.6 | 7.2 ± 0.8 |
| 21 | 60 | 1.58 | 57.2 ± 1.5 | 26.0 ± 1.8 | −54.8 ± 2.2 |
| 30 | 37 | 1.39 ± 0.13 | 56.2 ± 1.3 | 22.2 ± 0.3 | −60.5 ± 0.4 |
| 33 | 43 | 1.38 ± 0.25 | 54.9 ± 2.6 | 24.8 ± 1.4 | −54.5 ± 3.5 |

Results expressed as mean ± SEM, n = 1-2 (food intake) or n = 5-8 (body weight).

TABLE 6
Functional potencies at mouse-specific GLP-1R, GIPR,
and GcgR for select triple agonists of the invention.

| | mGLP-1R | mGIPR | mGcgR |
|---|---|---|---|
| | CRE Luc | CRE Luc | CRE Luc |
| Compound No. | EC₅₀ (pM) | EC₅₀ (pM) | EC₅₀ (pM) |
| hGLP-1(7-37) | 3.6 | no | nd |
| mGIP | nd | 29.6 | nd |
| hGlucagon | no | no | 4.4 |
| 21 | 2.9 | 9.8 | 56.1 |
| 30 | 2.6 | 11.2 | 5.7 |
| 33 | 2.2 | 10.0 | 8.1 |

nd = not determined.

An additional mouse study using Compound No.'s 26, 43, and 44 demonstrated superior reduction in food intake and body weight compared to vehicle in 010 mice after daily subcutaneous dosing over 13 days. Results are shown in Table 7. Compound No. 44 demonstrated a dose-dependent effect on reduction of food intake and body weight, and improved glucose tolerance compared to vehicle at the 1.0 nmol/kg and 3.0 nmol/kg doses. This demonstrates that weight lowering efficacy can be increased in a triple agonist without detrimental effects on glucose tolerance.

TABLE 7
Effects on food intake, body weight and glucose tolerance in DIO mice treated daily with vehicle
or a GLP-1/GIP/Gcg triple agonist at indicated doses.

| | Cumulative | | | Change in | |
|---|---|---|---|---|---|
| | food intake | Absolute BW | | BW | iAUC, IPGTT |
| Compound | (grams) | (grams) | | (%) | (min*mg/dL) |
| No. | Day 13 | Day 0 | Day 13 | Day 13 | Day 14 |
| Vehicle | 34.3 ± 0.6 | 59.5 ± 1.3 | 60.0 ± 1.3 | 0.8 ± 0.6 | 23256 ± 3864 |

0.3 nmol/kg

| | | | | | |
|---|---|---|---|---|---|
| 44 | 28.0 ± 0.6 | 59.2 ± 1.2 | 56.9 ± 1.5 | −3.9 ± 1.1 | 25356 ± 4338 |

1.0 nmol/kg

| | | | | | |
|---|---|---|---|---|---|
| 44 | 24.8 ± 1.8 | 60.8 ± 1.1 | 52.7 ± 1.3 | −13.4 ± 0.9 | 14606 ± 2410 |
| 26 | 20.1 ± 3.4 | 60.7 ± 1.9 | 51.6 ± 2.2 | −15.6 ± 0.9 | 8789 ± 1521 |
| 43 | 21.1 ± 3.3 | 60.4 ± 1.0 | 48.7 ± 1.4 | −19.4 ± 1.5 | nd |

3.0 nmol/kg

| | | | | | |
|---|---|---|---|---|---|
| 44 | 12.4 ± 1.5 | 60.6 ± 1.1 | 43.1 ± 1.8 | −29.1 ± 2.3 | 15161 ± 2257 |
| 43 | 11.5 ± 2.4 | 60.1 ± 1.9 | 36.3 ± 1.0 | −39.5 ± 1.3 | nd |

Results expressed as mean ± SEM, n = 2 (food intake) or n = 7-8 (body weight, IPGTT).
iAUC = baseline subtracted area under the curve.
nd = not determined.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A GLP-1/GIP/Gcg receptor triple agonist comprising a peptide and a substituent, wherein the amino acid sequence of the peptide is:
X₁X₂X₃GTFTSDYSX₁₂X₁₃LX₁₅KX₁₇X₁₈AX₂₀X₂₁FVX₂₄WLLX₂₈GGPSSGAPPPS (SEQ ID NO: 45), wherein
X₁ is H or Y
X₂ is Aib
X₃ is H or Q
X₁₂ is K, I or Y
X₁₃ is Y or L
X₁₅ is D or E
X₁₇ is Q, R, or K
X₁₈ is A, Y or K
X₂₀ is Aib, Q, E, R, or H
X₂₁ is E or K
X₂₄ is N or Q
X₂₈ is A or E;
wherein the peptide is a C-terminal acid or amide;
wherein the substituent is A-B-C-;
wherein A- is HOOC—(CH₂)ₚ—CO—* (Chem. 1), wherein p is an integer in the range of 16-20;
wherein B- is *—NH—CH(COOH)—(CH₂)₂—CO—* (Chem. 2) or *—NH—CH₂—(C₆H₁₀)—CO—NH—CH(COOH)—(CH₂)₂—C—-* (Chem. 3);
wherein C- is *—[NH—((CH₂)₂—O)₂—CH₂—CO]₂—* (Chem. 4) or *—[NH—(CH₂)₄—CH(NH₂)—CO]₂—* (Chem. 5);
wherein * denotes the points of attachment and A, B, and C are interconnected by amide bonds in the sequence indicated; and
wherein the substituent is covalently attached to the epsilon-amino group of the K in a position selected from the group consisting of positions 16, 17, and 21;
or pharmaceutically acceptable salts thereof.

2. The GLP-1/GIP/Gcg receptor triple agonist according to claim 1, wherein the substituent is covalently attached to the epsilon-amino group of K in position 16.

3. The GLP-1/GIP/Gcg receptor triple agonist according to claim 1, wherein X₁X₂X₃ is HAibH or YAibQ.

4. The GLP-1/GIP/Gcg receptor triple agonist according to claim 1, wherein SX₁₂X₁₃LX₁₅ is selected from the group consisting of SYYLE (SEQ ID NO: 54), SILLE (SEQ ID NO: 55), SYLLE (SEQ ID NO: 56), SIYLE (SEQ ID NO: 57), and SIYLD (SEQ ID NO: 58).

5. The GLP-1/GIP/Gcg receptor triple agonist according to claim 1, wherein KX₁₇X₁₈AX₂₀ is selected from the group consisting of KQAAAib (SEQ ID NO: 59), KKAAAib (SEQ ID NO: 60), KQYAAib (SEQ ID NO: 61), and KKYAAib (SEQ ID NO: 62).

6. The GLP-1/GIP/Gcg receptor triple agonist according to claim 1, wherein the amino acid sequence of the peptide is:
HX₂HGTFTSDYSX₁₂X₁₃LX₁₅KX₁₇X₁₈AX₂₀EFVX₂₄WLLX₂₈GGPSSGAPPPS (SEQ ID NO: 47), wherein
X₂ is Aib
X₁₂ is I or Y
X₁₃ is Y or L
X₁₅ is D or E
X₁₇ is Q or K
X₁₈ is A or Y
X₂₀ is Aib
X₂₄ is N or Q
X₂₈ is A or E.

7. The GLP-1/GIP/Gcg receptor triple agonist according to claim 1, wherein the amino acid sequence of the peptide is:
YX₂QGTFTSDYSX₁₂X₁₃LX₁₅KX₁₇X₁₈AX₂₀EFVX₂₄WLLX₂₈GGPSSGAPPPS (SEQ ID NO: 50), wherein
X₂ is Aib
X₁₂ is I or Y
X₁₃ is Y or L
X₁₅ is D or E
X₁₇ is Q or K
X₁₈ is A or Y
X₂₀ is Aib
X₂₄ is N or Q
X₂₈ is A or E.

8. (canceled)

9. The GLP-1/GIP/Gcg receptor triple agonist according to claim 1, wherein the substituent A-B-C- is selected from the group consisting of:

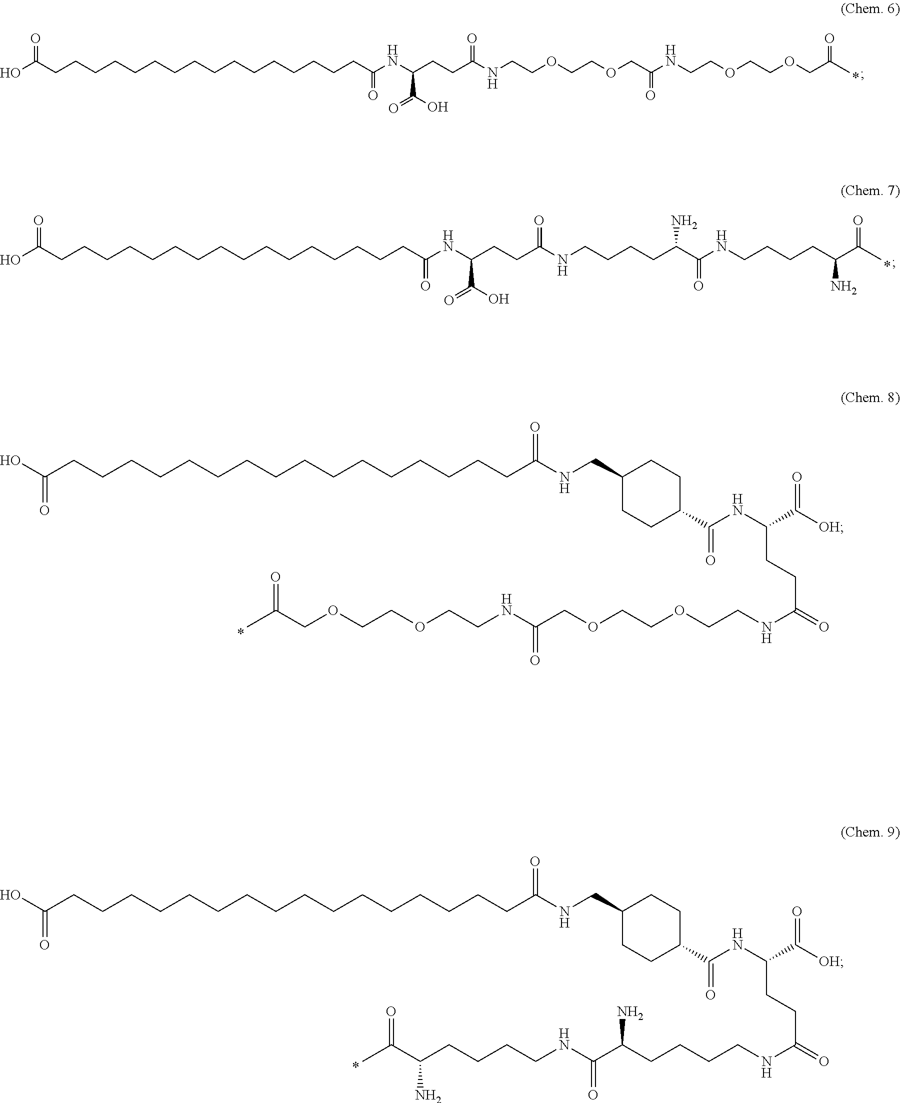 [SMILES: CC(=O)COCCOCCNC(=O)COCCOCCNC(=O)CC[C@H](NC(=O)CCCCCCCCCCCCCCCCC(=O)O)C(=O)O ; CC(=O)[C@@H](N)CCCCNC(=O)[C@@H](N)CCCCNC(=O)CC[C@H](NC(=O)CCCCCCCCCCCCCCCCC(=O)O)C(=O)O ; CC(=O)COCCOCCNC(=O)COCCOCCNC(=O)CC[C@H](NC(=O)[C@H]1CC[C@H](CNC(=O)CCCCCCCCCCCCCCCCC(=O)O)CC1)C(=O)O ; CC(=O)[C@@H](N)CCCCNC(=O)[C@@H](N)CCCCNC(=O)CC[C@H](NC(=O)[C@H]1CC[C@H](CNC(=O)CCCCCCCCCCCCCCCCC(=O)O)CC1)C(=O)O]

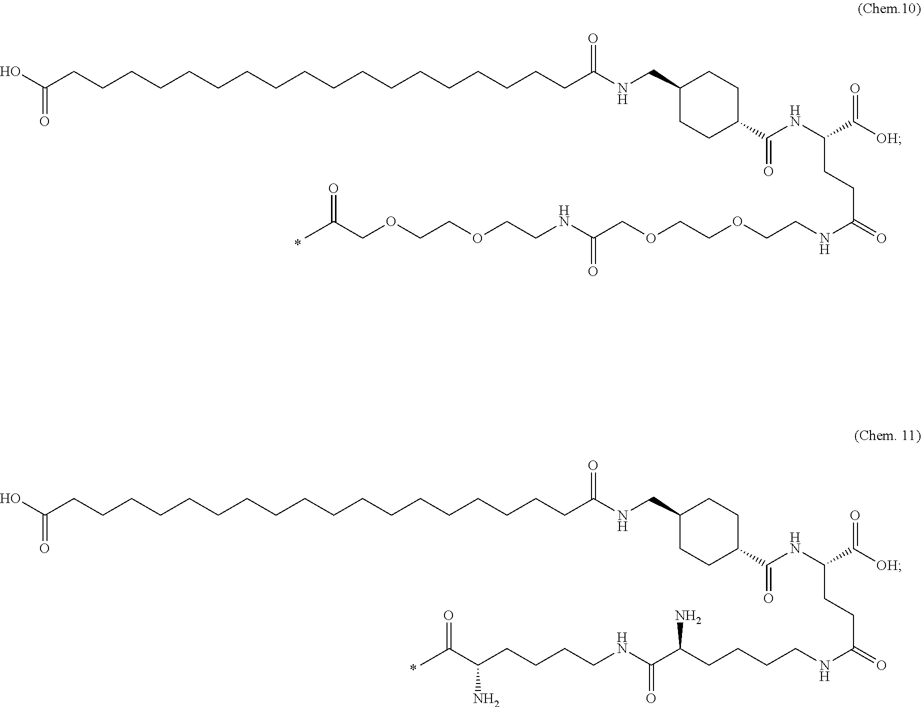 [SMILES: CC(=O)COCCOCCNC(=O)COCCOCCNC(=O)CC[C@H](NC(=O)[C@H]1CC[C@H](CNC(=O)CCCCCCCCCCCCCCCCCCC(=O)O)CC1)C(=O)O ; CC(=O)[C@@H](N)CCCCNC(=O)[C@@H](N)CCCCNC(=O)CC[C@H](NC(=O)[C@H]1CC[C@H](CNC(=O)CCCCCCCCCCCCCCCCCCC(=O)O)CC1)C(=O)O]

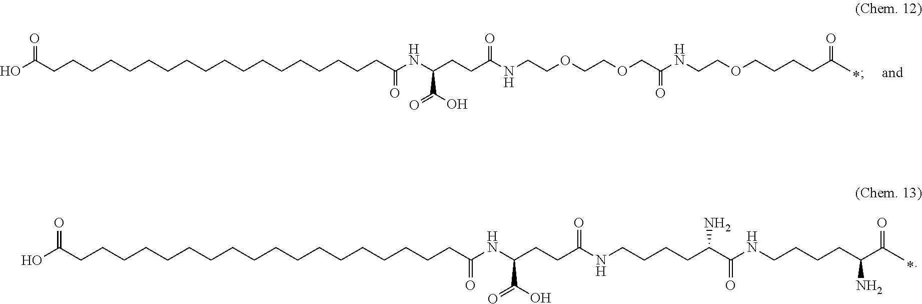 [SMILES: CC(=O)CCCCOCCNC(=O)COCCOCCNC(=O)CC[C@H](NC(=O)CCCCCCCCCCCCCCCCCCC(=O)O)C(=O)O ; CC(=O)[C@@H](N)CCCCNC(=O)[C@@H](N)CCCCNC(=O)CC[C@H](NC(=O)CCCCCCCCCCCCCCCCCCC(=O)O)C(=O)O]

10. The GLP-1/GIP/Gcg receptor triple agonist according to claim 1, wherein triple agonist is selected from the group consisting of:
Y-Aib-QGTFTSDYSIYLD-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-QAA-Aib-EFVNWLLAGGPSSGAPPPS-NH₂ (Compound No. 1): 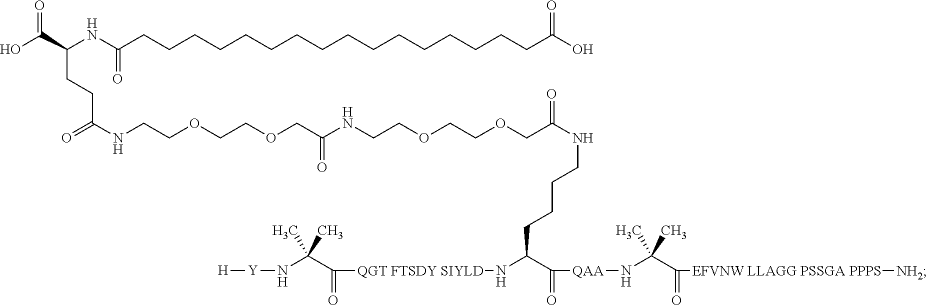
Y-Aib-QGTFTSDYSIYLDK-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-AA-Aib-EFVNWLLAGGPSSGAPPPS-NH₂ (Compound No. 2): 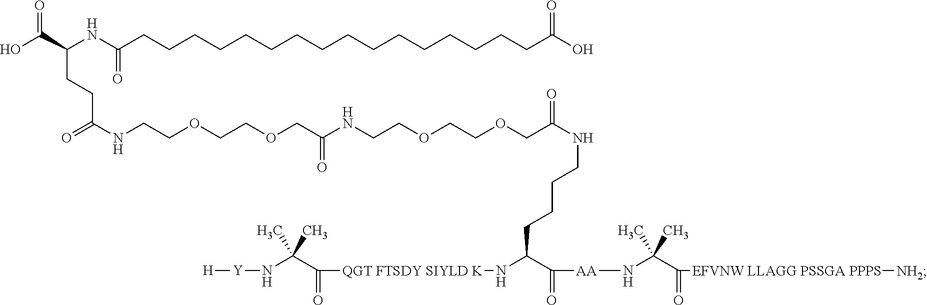
Y-Aib-QGTFTSDYSIYLDKQAA-Aib-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-FVNWLLAGGPSSGAPPPS-NH₂ (Compound No. 3): 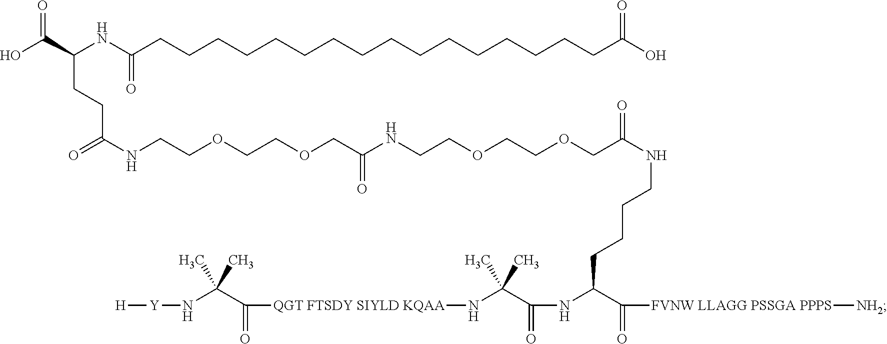
Y-Aib-QGTFTSDYSIYLDKQAA-Aib-K[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-FVQWLLEGGPSSGAPPPS-NH₂ (Compound No. 4): 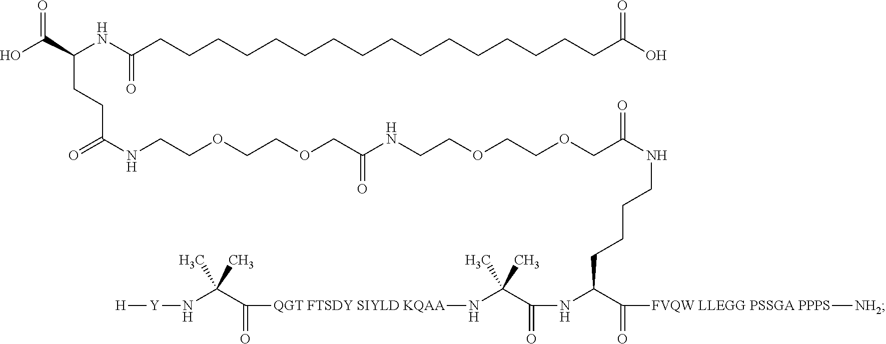
Y-Aib-QGTFTSDYSIYLD-K[2-[2-[2-[2-[2-[2-[[[(4S)-4-carboxy-4-(19-carboxynonadecanoylamino) butanoyl]amino]ethoxy]ethoxy]acetyl]amino]ethoxy]ethoxy]acetyl]-QAA-Aib-EFVNWLLAGGPSSGAPPPS-NH2 (Compound No. 5): 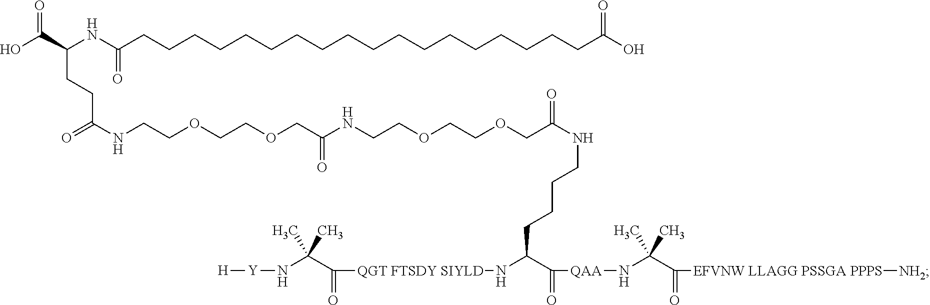
Y-Aib-QGTFTSDYSIYLD-K[2-[2-[2-[[2-[2-[2-[[(4S)-4-carboxy-4-[[4-[(19-carboxynonadecanoylamino) methyl]cyclohexanecarbonyl]amino]butanoyl]amino]ethoxy ]ethoxy]acetyl]amino]ethoxy]ethoxy]acetyl]-QAA-Aib-EFVNWLLAGGPSSGAPPPS-NH2(Compound No. 6): 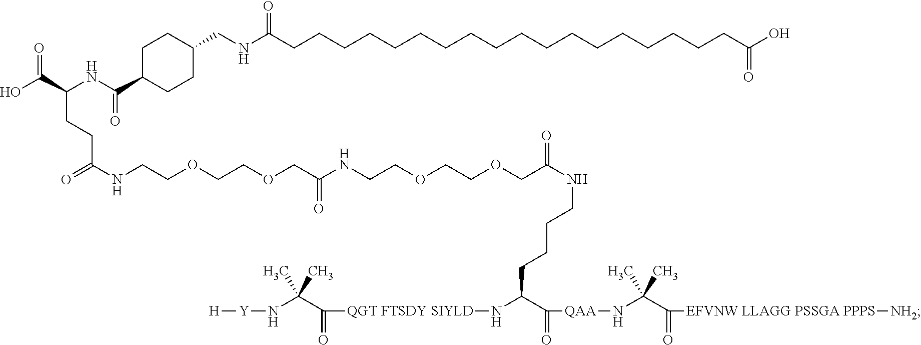
Y-Aib-QGTFTSDYSIYLDKQAA-Aib-K[2-[2-[2-[[2-[2-[2-11(4S)-4-carboxy-4-(19-carboxynonadecanoylamino)butanoyl]amino]ethoxy]ethoxy]acetyl]amino]ethoxy]ethoxy]acetyl]-FVQWLLEGGPSSGAPPPS-NH₂ (Compound No. 7): 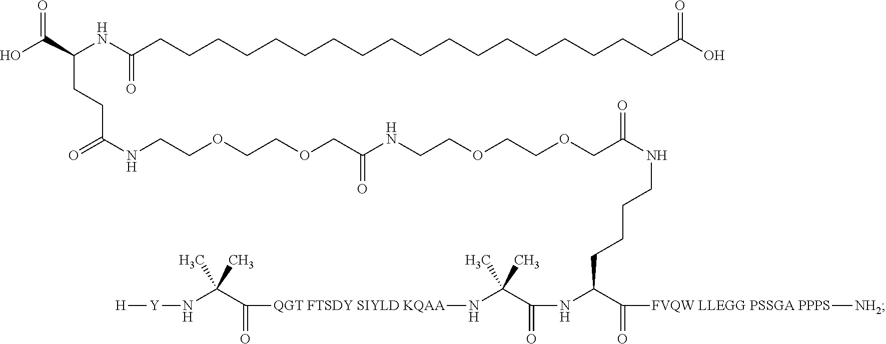
Y-Aib-QGTFTSDYSIYLDKQAA-Aib-K[2-[2-[2-[[2-[2-[2-[[(4S)-4-carboxy-4-[[4-[(19-carboxynonadecanoylamino)methyl]cyclohexanecarbonyl]amino]butanoyl]amino]ethoxy]ethoxy]acetyl]amino]ethoxy]ethoxy]acetyl]-FVQWLLEGGPSSGAPPPS-NH₂ (Compound No. 8): 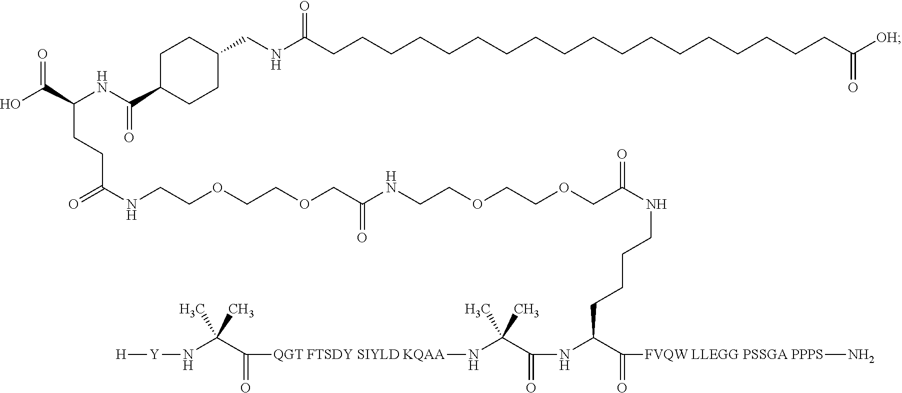
Y-Aib-QGTFTSDYSIYLDK-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-AA-Aib-EFVNWLLAGGPSSGAPPPS-NH₂ (Compound No. 9): 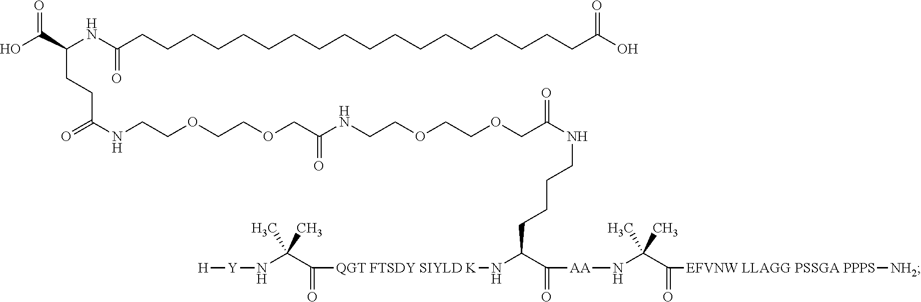
Y-Aib-QGTFTSDYSIYLDK-K[2-[2-[2-[[2-[2-[2-[[(4S)-4-carboxy-4-[[4-[(19-carboxynonadecanoylamino)methyl]cyclohexanecarbonyl]amino]butanoyl]amino]ethoxy]ethoxy]acetyl]amino]ethoxy]ethoxy]acetyl]-AA-Aib-EFVNWLLAGGPSSGAPPPS-NH₂ (Compound No. 10): 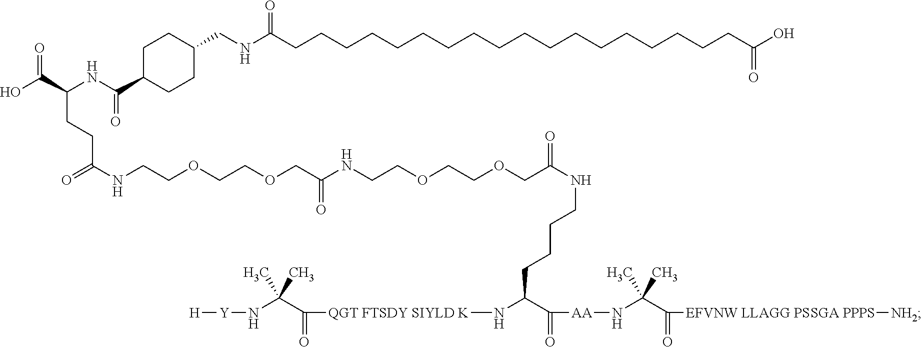
Y-Aib-QGTFTSDYSIYLEKQAA-Aib-K[2-[2-[2-[[2-[2-[2-[[(4S)-4-carboxy-4-14-1(19-carboxynonadecanoylamino)methyl]cyclohexanecarbonyl]amino]butanoyl]amino]ethoxy]ethoxy]acetyl]amino]ethoxy]ethoxy]acetyl]-FVQWLLEGGPSSGAPPPS-NH₂ (Compound No. 11): 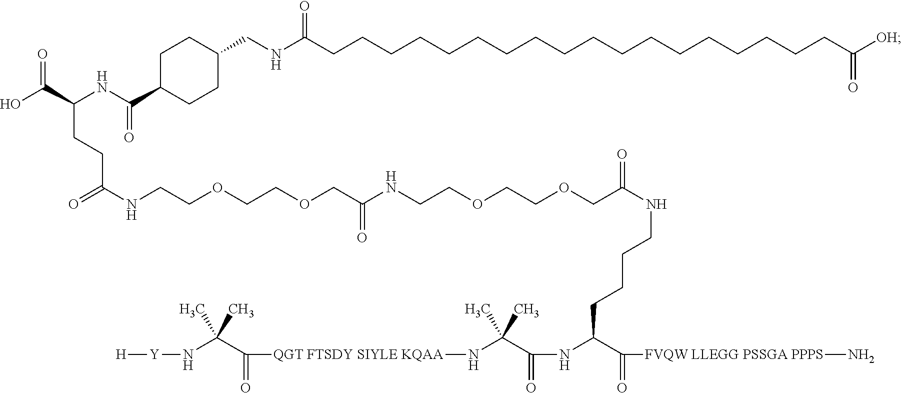
Y-Aib-QGTFTSDYSIYLE-K[2-[2-[2-[[2-[2-[2-[[(4S)-4-carboxy-4-[[4-[(19-carboxynonadecanoylamino)methyl]cyclohexanecarbonyl]amino]butanoyl]amino]ethoxy]ethoxy]acetyl]amino]ethoxy]ethoxy]acetyl]-QAA-Aib-EFVNWLLAGGPSSGAPPPS-NH₂ (Compound No. 12): 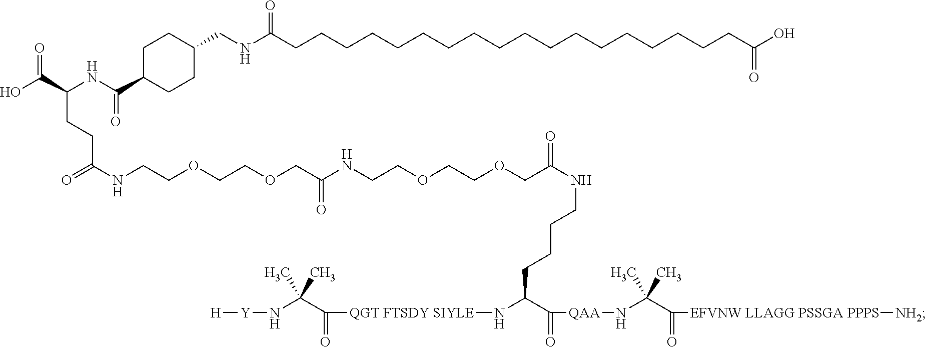
Y-Aib-QGTFTSDYSIYLE-K[(2S)-2-amino-6-[[(2S)-2-amino-6-[[(4S)-4-carboxy-4-[[4-[(19-carboxynonadecanoylamino)methyl]cyclohexanecarbonyl]amino]butanoyl]amino]hexanoyl]amino]hexanoyl]-QAA-Aib-EFVNWLLAGGPSSGAPPPS-NH₂ (Compound No. 13): 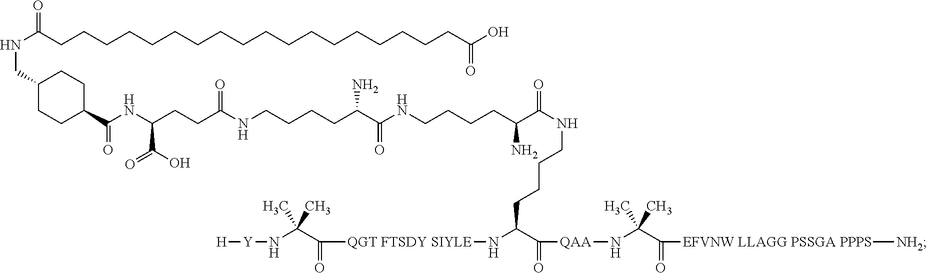
Y-Aib-QGTFTSDYSIYLE-K[2-[2-[2-[[2-[2-[2-[[(4S)-4-carboxy-4-[[4-[(19-carboxynonadecanoylamino)methyl]cyclohexanecarbonyl]amino]butanoyl]amino]ethoxy]ethoxy]acetyl]amino]ethoxy]ethoxy]acetyl]-QAA-Aib-EFVQWLLEGGPSSGAPPPS-NH₂ (Compound No. 14): 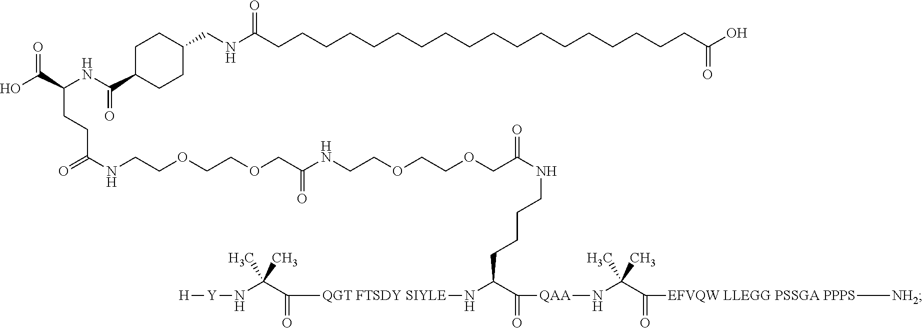
Y-Aib-QGTFTSDYSIYLE-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-QAA-Aib-EFVNWLLAGGPSSGAPPPS-NH₂ (Compound No. 15): 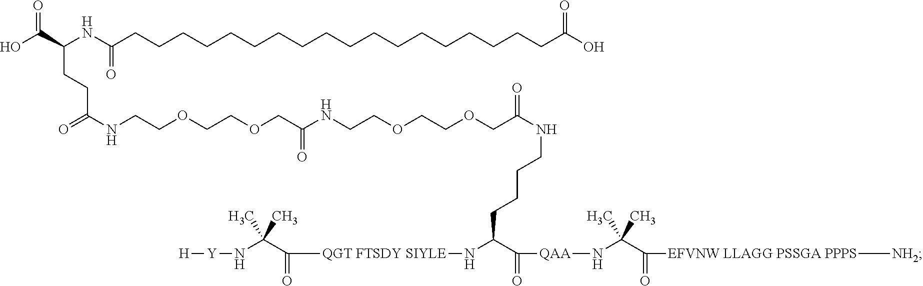
Y-Aib-QGTFTSDYSIYLE-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-QAA-Aib-EFVQWLLEGGPSSGAPPPS-NH₂ (Compound No. 16): 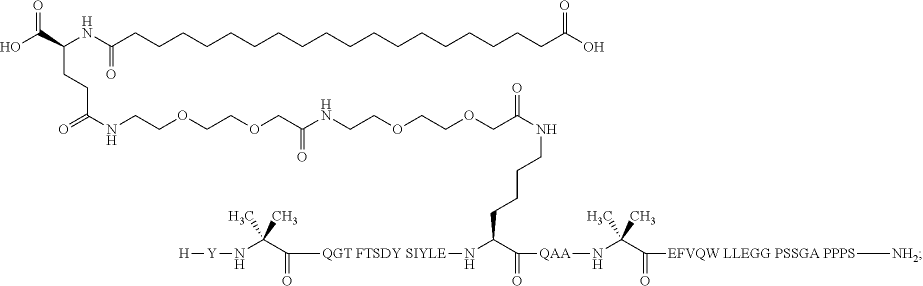
Y-Aib-QGTFTSDYSIYLE-K[(2S)-[2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂ (Compound No. 17): 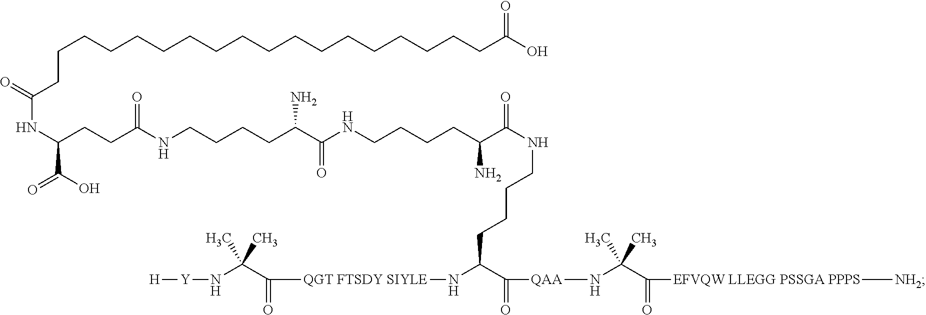
Y-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂ (Compound No. 18): 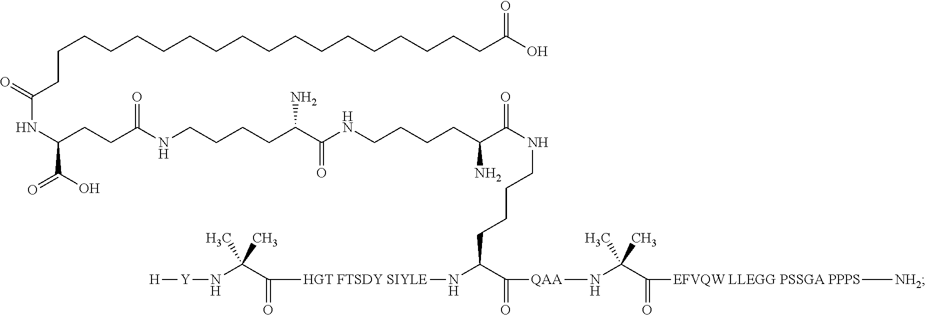
H-Aib-QGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂ (Compound No. 19): 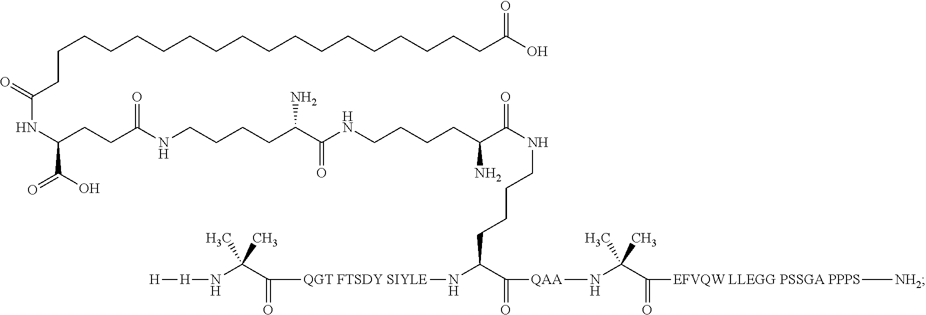
Y-Aib-QGTFTSDYSIYLE-K[2-[2-[2-[[2-[2-[2-[[(4S)-4-carboxy-4-[[4-[(19-carboxynonadecanoylamino)methyl]cyclohexanecarbonyl]amino]butanoyl]amino]ethoxy]ethoxy]acetyl]amino]ethoxy]ethoxy]acetyl]-QAAQEFVQWLLEGGPSSGAPPPS-NH₂ (Compound No. 20): 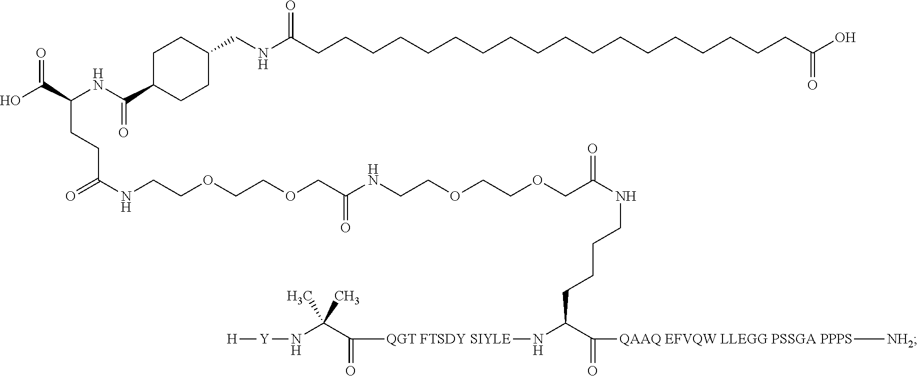
H-Aib-HGTFTSDYSIYLE-K[(2S)-2-amino-6-[[(2S)-2-amino-6-[[(4S)-4-carboxy-4-[[4-[(19-carboxynonadecanoylamino)methyl]cyclohexanecarbonyl]amino]butanoyl]amino]hexanoyl]amino]hexanoyl]-QAA-Aib-EFVQWLLEGGPSSGAPPPS-NH₂ (Compound No. 21): 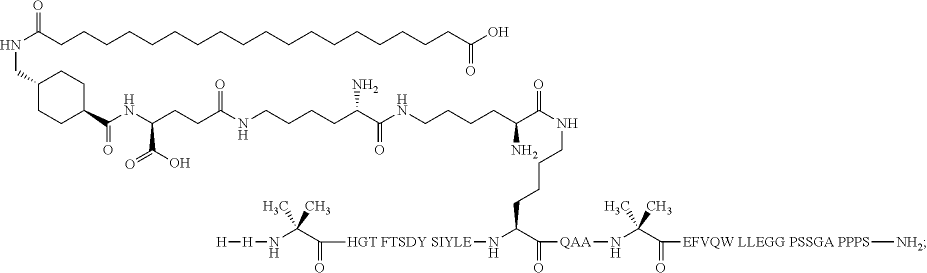
H-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂ (Compound No. 22): 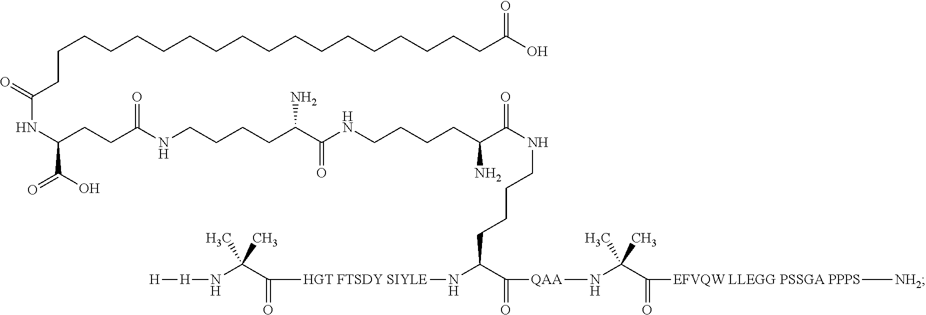
H-Aib-HGTFTSDYSIYLE-K[(2S)-2-amino-6-[[(2S)-2-amino-6-[[(4S)-4-carboxy-4-[[4-[(19-carboxynonadecanoylamino)methyl]cyclohexanecarbonyl]amino]butanoyl]amino]hexanoyl]amino]hexanoyl]-QAAQEFVQWLLEGGPSSGAPPPS-NH₂(Compound No. 23): 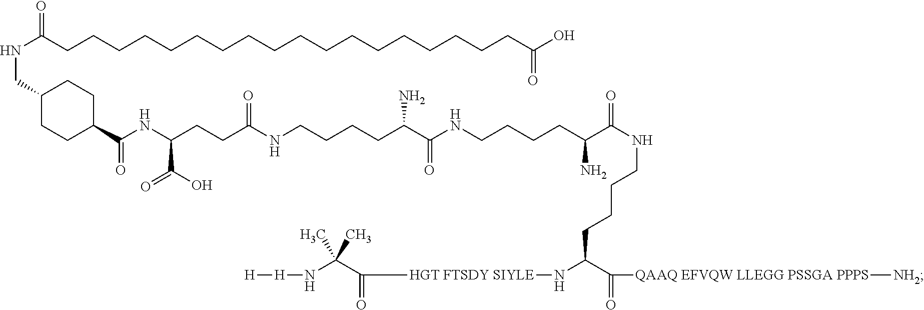
H-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QYA-Aib-EFVQWLLEGGPSSGAPPPS-NH₂ (Compound No. 24): 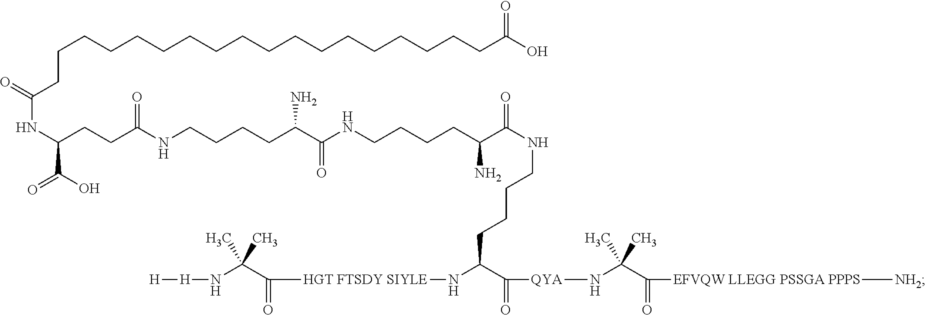
H-Aib-HGTFTSDYSYYLE-K[(2S)-2-amino-6-[[(2S)-2-amino-6-[[(4S)-4-carboxy-4-[[4-[(19-carboxynonadecanoylamino)methyl]cyclohexanecarbonyl]amino]butanoyl]amino]hexanoyl]amino]hexanoyl]-QAA-Aib-EFVQWLLEGGPSSGAPPPS-NH₂ (Compound No. 25): 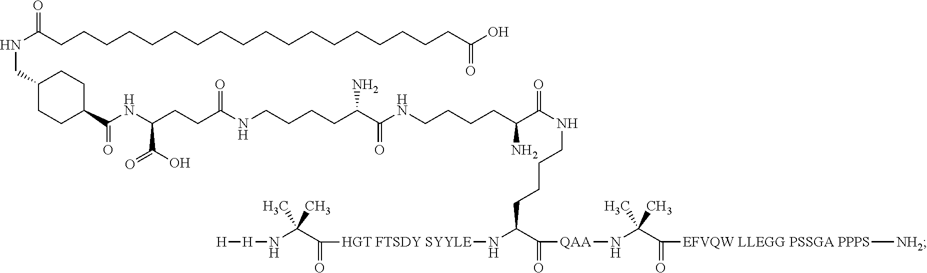
H-Aib-HGTFTSDYSYYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂ (Compound No. 26): 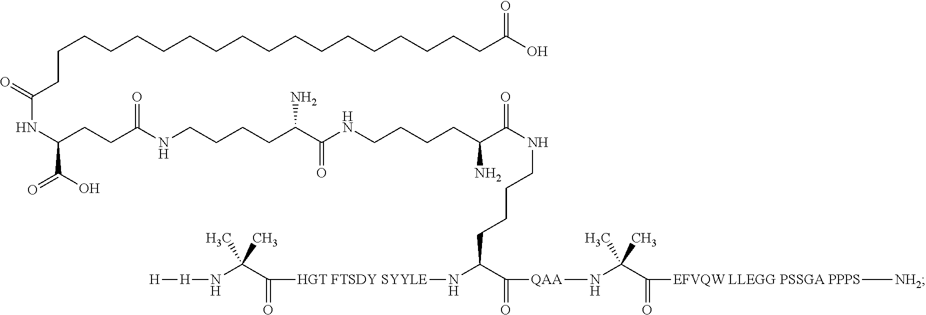
H-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAAEEFVQWLLEGGPSSGAPPPS-NH₂ (Compound No. 27): 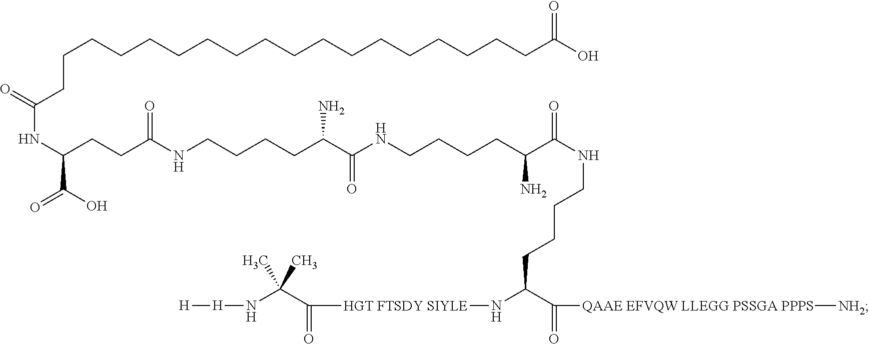
H-Aib-HGTFTSDYSIYLD-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂ (Compound No. 28): 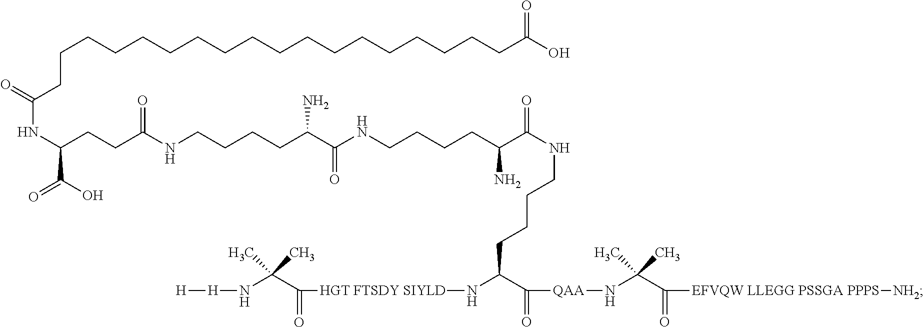
H-Aib-HGTFTSDYSYYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QYA-Aib-EFVQWLLEGGPSSGAPPPS-NH₂ (Compound No. 29): 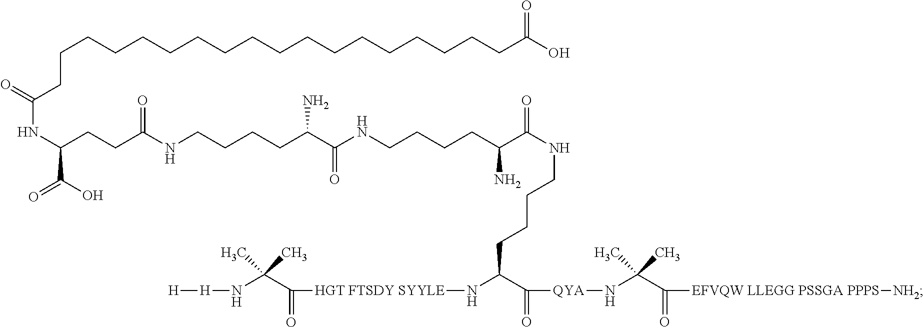
H-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-KYA-Aib-EFVQWLLEGGPSSGAPPPS-NH₂ (Compound No. 30): 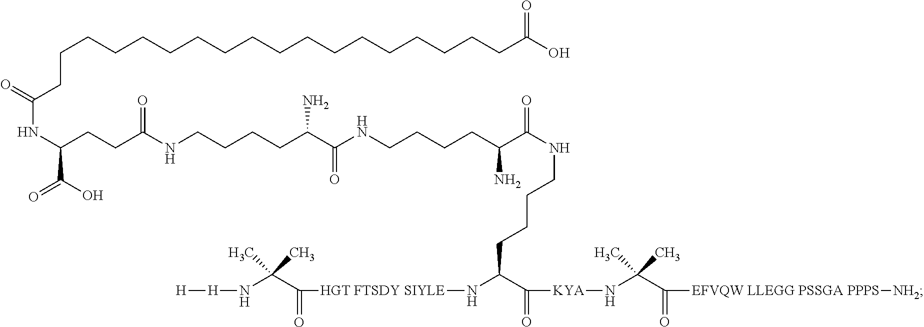
H-Aib-HGTFTSDYSYYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-KAA-Aib-EFVQWLLEGGPSSGAPPPS-NH₂ (Compound No. 31): 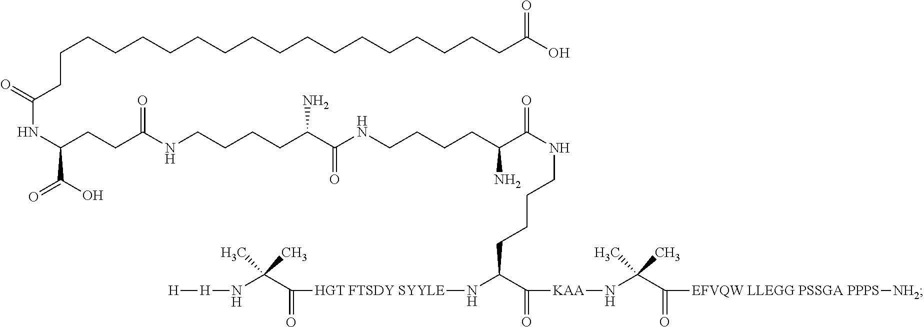
H-Aib-HGTFTSDYSYYLE-K[(2S)-2-amino-6-[[(2S)-2-amino-6-[[(4S)-4-carboxy-4-(17-carboxyheptadecanoylamino)butanoyl]amino]hexanoyl]amino]hexanoyl]-QAA-Aib-EFVQWLLEGGPSSGAPPPS-NH₂ (Compound No. 32): 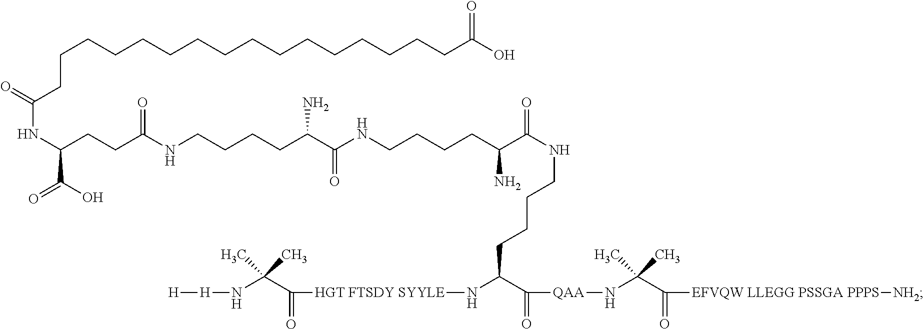
H-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-KAA-Aib-EFVQWLLEGGPSSGAPPPS-NH₂ (Compound No. 33): 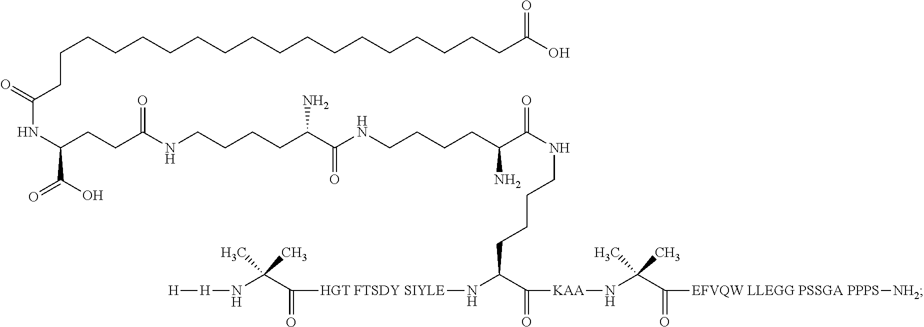
H-Aib-HGTFTSDYSIYLE-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-KAA-Aib-EFVQWLLEGGPSSGAPPPS-NH₂ (Compound No. 34): 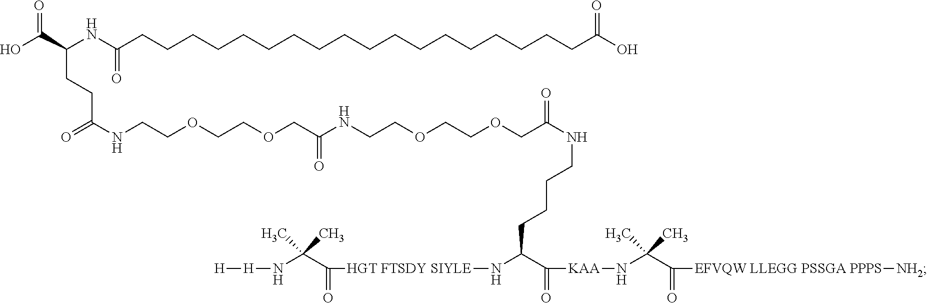
H-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QKA-Aib-EFVQWLLEGGPSSGAPPPS-NH₂ (Compound No. 35): 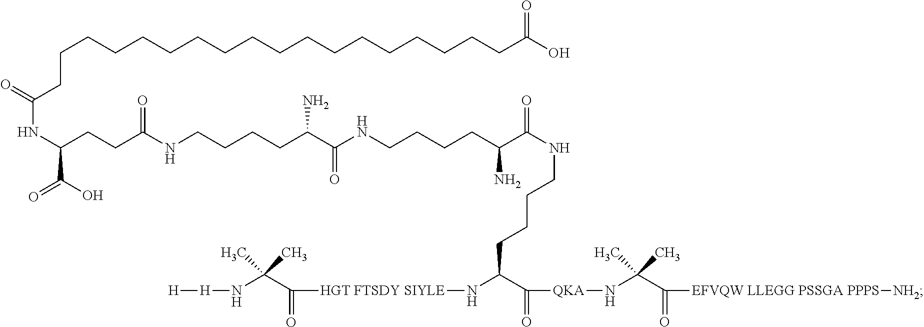
H-Aib-HGTFTSDYSKYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂ (Compound No. 36): 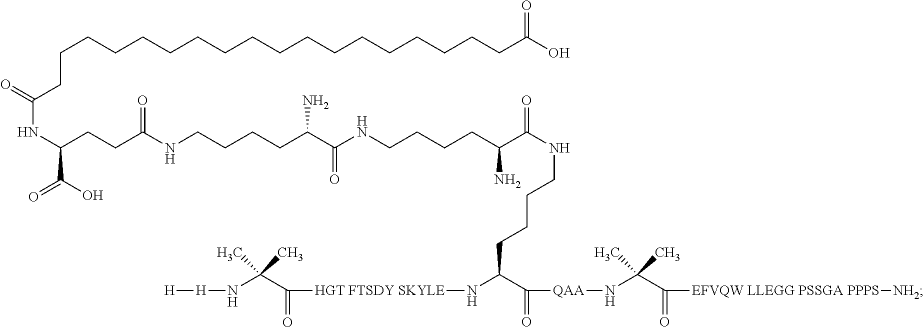
H-Aib-HGTFTSDYSIYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-RAA-Aib-EFVQWLLEGGPSSGAPPPS-NH₂ (Compound No. 37): 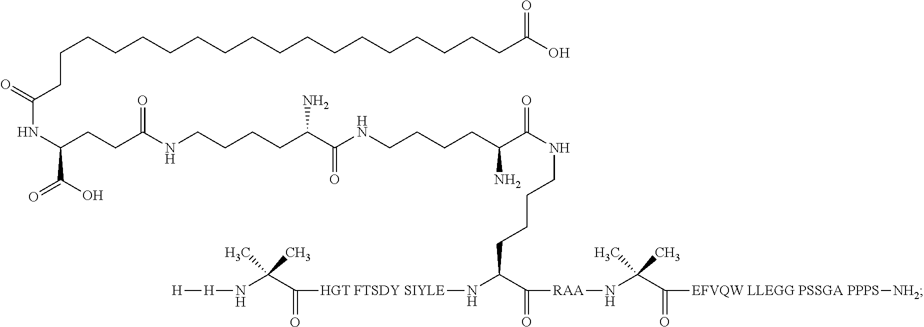
H-Aib-HGTFTSDYSIYLE-K[(2S)-2-amino-6-[[(2S)-2-amino-6-[[(4S)-4-carboxy-4-(17-carboxyheptadecanoylamino)butanoyl]amino]hexanoyl]amino]hexanoyl]-KYA-Aib-EFVQWLLEGGPSSGAPPPS-NH₂ (Compound No. 38): 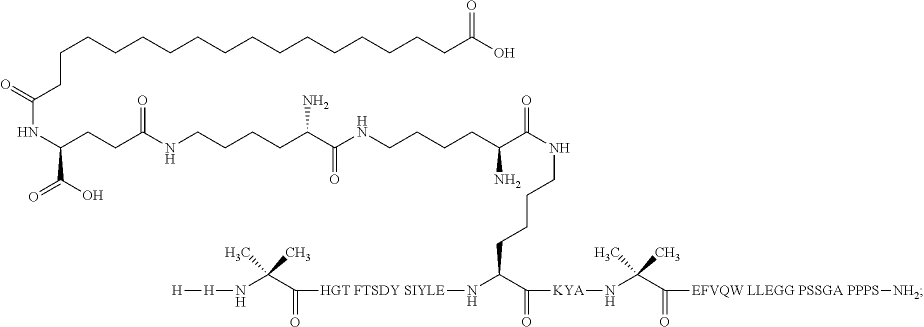
H-Aib-HGTFTSDYSIYLE-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-KYA-Aib-EFVQWLLEGGPSSGAPPPS-NH₂ (Compound No. 39): 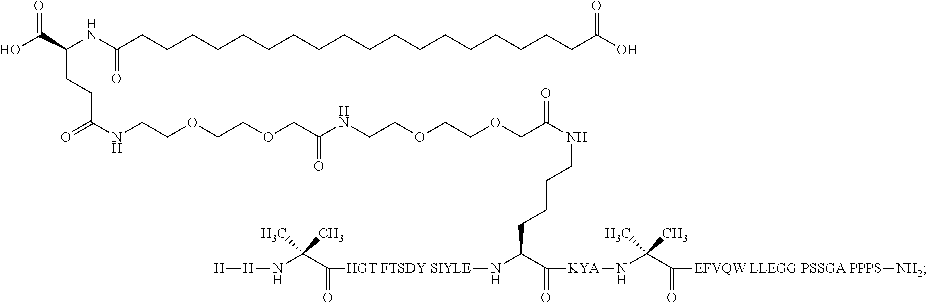
H-Aib-HGTFTSDYSYYLE-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-QAA-Aib-EFVQWLLEGGPSSGAPPPS-NH₂ (Compound No. 40): 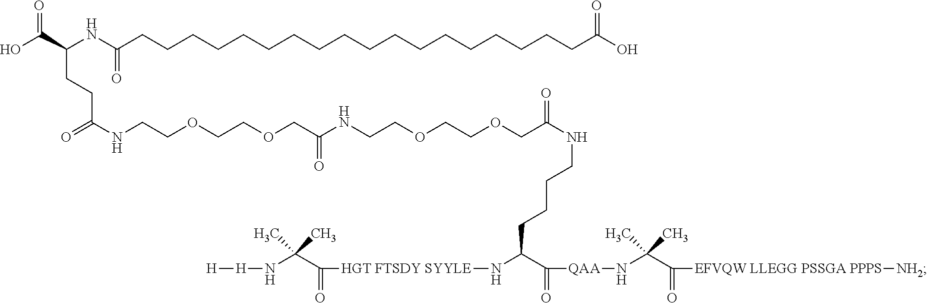
H-Aib-HGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-KYA-Aib-EFVQWLLEGGPSSGAPPPS-NH₂ (Compound No. 41): 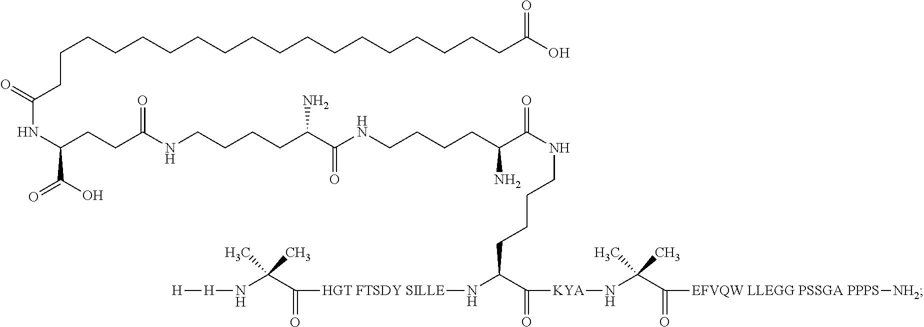
H-Aib-HGTFTSDYSYLLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂ (Compound No. 42): 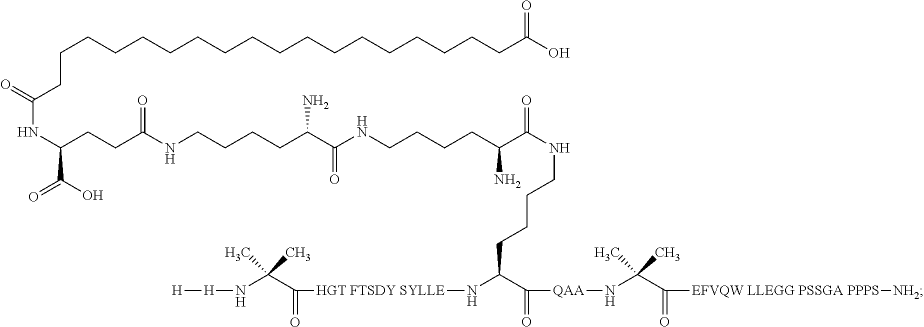
H-Aib-HGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂ (Compound No. 43): 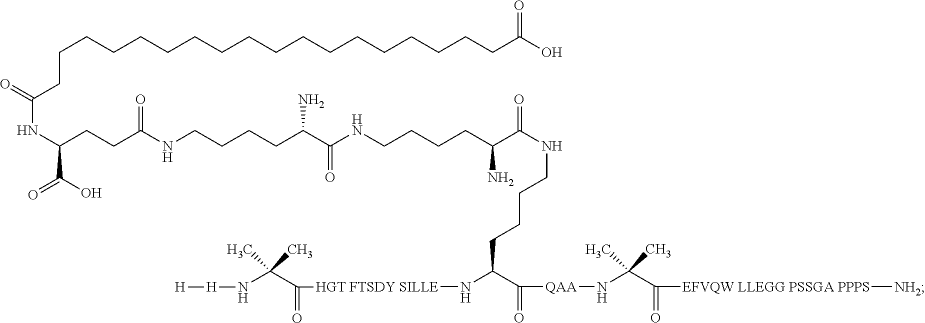
H-Aib-HGTFTSDYSIYLE-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-QAA-Aib-EFVQWLLEGGPSSGAPPPS-NH₂ (Compound No. 44): 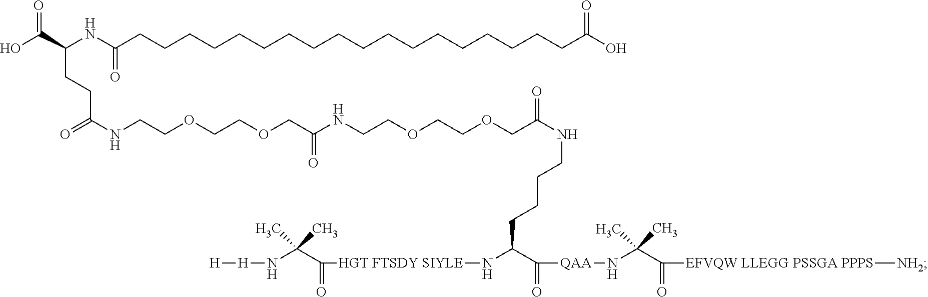
H-Aib-HGTFTSDYSILLE-K[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]-QAA-Aib-EFVQWLLEGGPSSGAPPPS-NH₂(Compound No. 45): 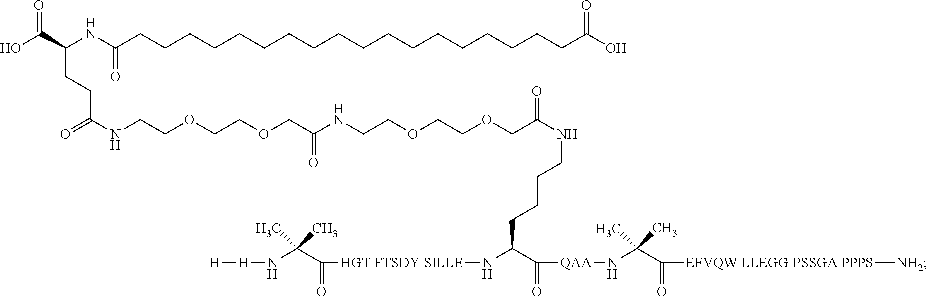
Y-Aib-QGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂ (Compound No. 46): 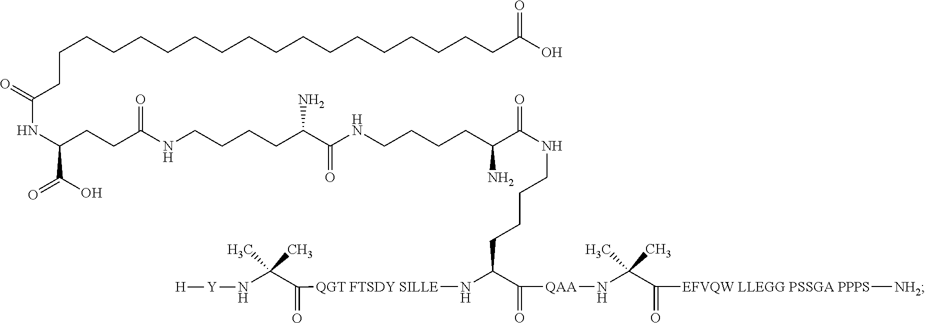
Y-Aib-QGTFTSDYSYYLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂ (Compound No. 47): 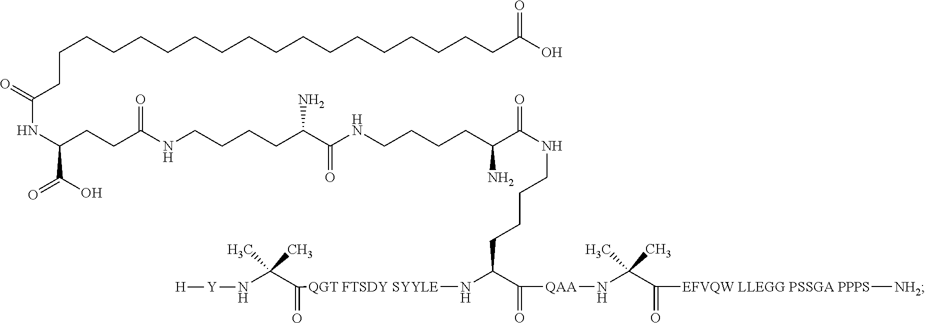
Y-Aib-QGTFTSDYSYLLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAA-Aib-EFVQWLLEGGPSSGAPPPS-NH₂ (Compound No. 48): 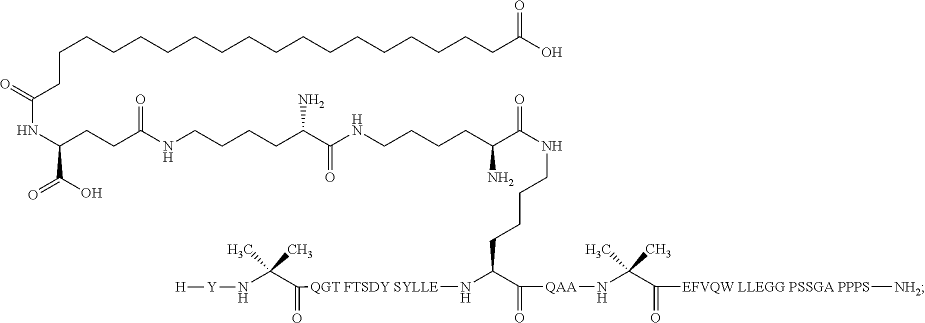
H-Aib-HGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAAEEFVQWLLEGGPSSGAPPPS-NH₂ (Compound No. 49): 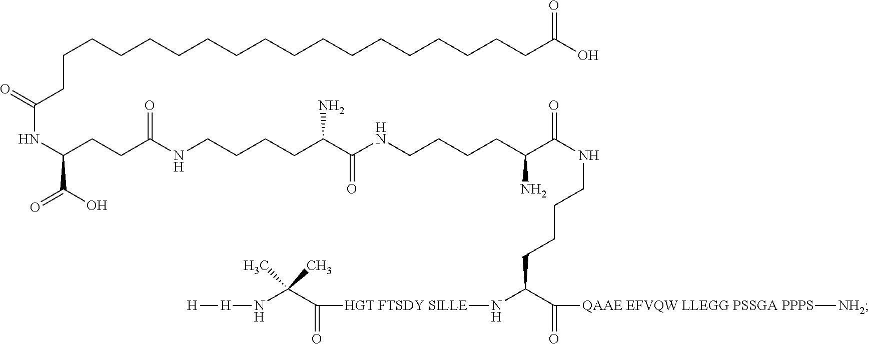
H-Aib-HGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAAEEFVQWLLEGGPSSGAPPPS-OH (Compound No. 50): 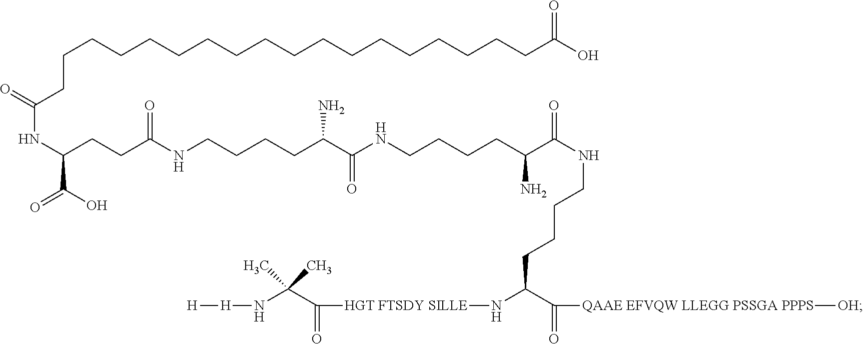
H-Aib-HGTFTSDYSILLE-K[(2S)-2-amino-6-[[(2S)-2-amino-6-[[(4S)-4-carboxy-4-(17-carboxyheptadecanoylamino)butanoyl]amino]hexanoyl]amino]hexanoyl]-QAAEEFVQWLLEGGPSSGAPPPS-OH (Compound No. 51): 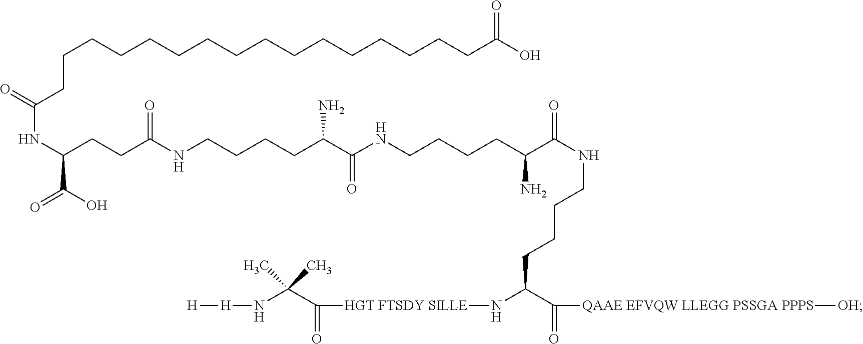
H-Aib-HGTFTSDYSILLE-K[(2S)-2-amino-6-[[(2S)-2-amino-6-[[(4S)-4-carboxy-4-(17-carboxyheptadecanoylamino)butanoyl]amino]hexanoyl]amino]hexanoyl]-QAAQEFVQWLLEGGPSSGAPPPS-OH (Compound No. 52): 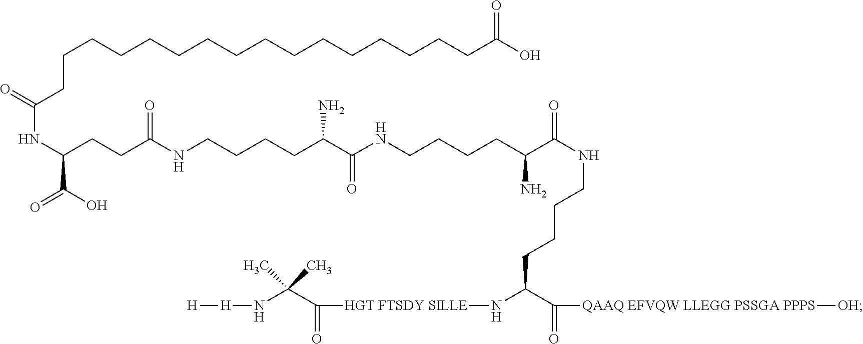
H-Aib-HGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAAQEFVQWLLEGGPSSGAPPPS-OH (Compound No. 53): 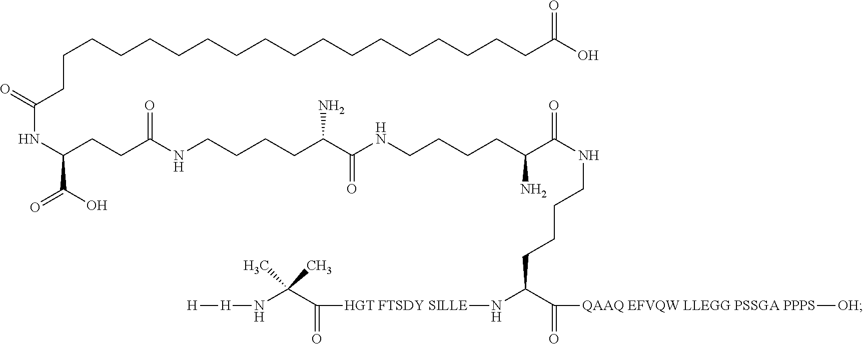
H-Aib-HGTFTSDYSILLE-K[(2S)-2-amino-6-[[(2S)-2-amino-6-[[(4S)-4-carboxy-4-(17-carboxyheptadecanoylamino)butanoyl]amino]hexanoyl]amino]hexanoyl]-QAAREFVQWLLEGGPSSGAPPPS-OH (Compound No. 54): 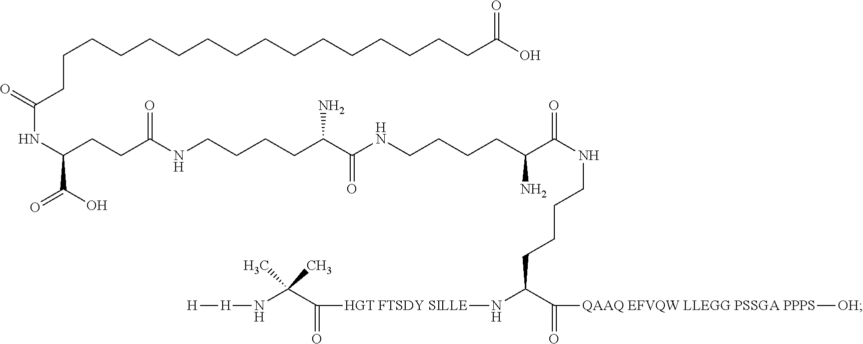
H-Aib-HGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAAREFVQWLLEGGPSSGAPPPS-OH (Compound No. 55): 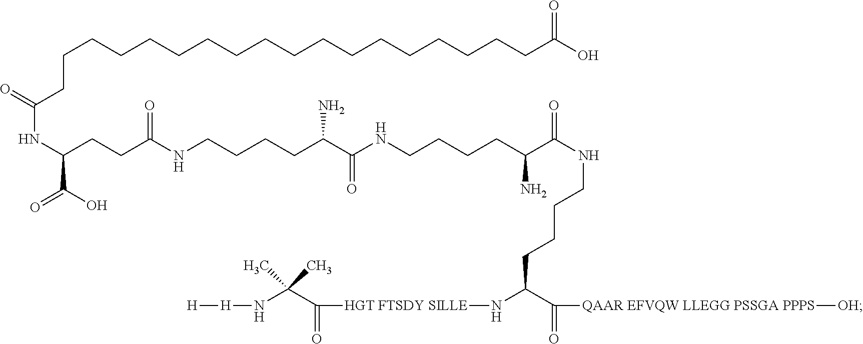
H-Aib-HGTFTSDYSILLE-K[(2S)-2-amino-6-[[(2S)-2-amino-6-[[(4S)-4-carboxy-4-(17-carboxyheptadecanoylamino)butanoyl]amino]hexanoyl]amino]hexanoyl]-QAAHEFVQWLLEGGPSSGAPPPS-OH (Compound No. 56): 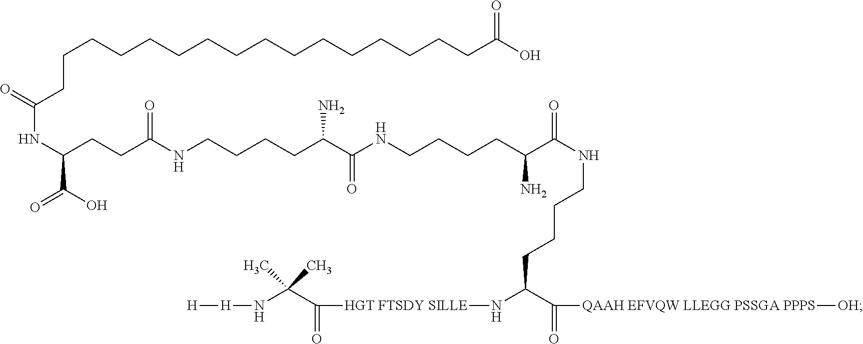
and
H-Aib-HGTFTSDYSILLE-K[(2S)-2-amino-6-[[(2S)-2-amino-6-[[(4S)-4-carboxy-4-(19-carboxynonadecanoylamino)butanoyl]amino]hexanoyl]amino]hexanoyl]-QAAHEFVQWLLEGGPSSGAPPPS-OH (Compound No. 57):

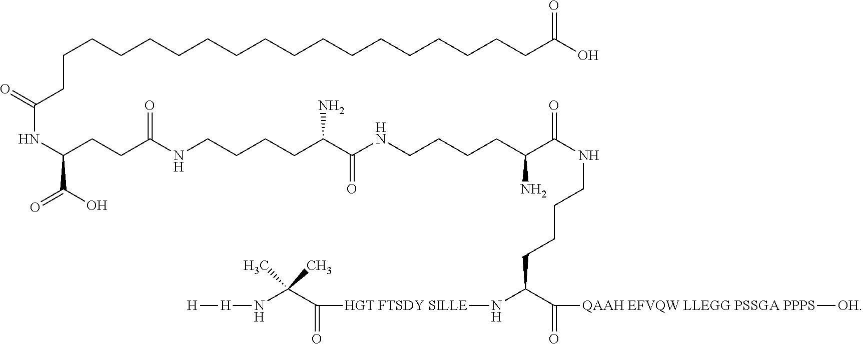

11. A pharmaceutical composition comprising the GLP-1/GIP/Gcg receptor triple agonist according to claim 1, and optionally at least one pharmaceutically acceptable excipient.

12. A method for reducing body weight, comprising administering the GLP-1/GIP/Gcg receptor triple agonist according to claim 1 to a subject in need thereof.

13. The method according to claim 12, wherein the subject is suffering from obesity.

14.-15. (canceled)

16. A method for reducing body weight, comprising administering the GLP-1/GIP/Gcg receptor triple agonist according to claim 6.

17. A method for reducing body weight, comprising administering the GLP-1/GIP/Gcg receptor triple agonist according to claim 7.
